(19) United States
(12) Patent Application Publication
(10) Pub. No.: US 2025/0295025 A1
(43) Pub. Date: Sep. 18, 2025

(54) ORGANIC COMPOUND AND ORGANIC LIGHT EMITTING DEVICE COMPRISING SAME

(71) Applicant: LG CHEM, LTD., Seoul (KR)

(72) Inventors: Jae Seung HA, Daejeon (KR); Sujeong GEUM, Daejeon (KR); Woochul LEE, Daejeon (KR); Jiye JEON, Daejeon (KR); Woo Jin CHO, Daejeon (KR); Ji Young CHOI, Daejeon (KR)

(21) Appl. No.: 19/074,258

(22) Filed: Mar. 7, 2025

(30) Foreign Application Priority Data
Mar. 14, 2024 (KR) 10-2024-0035510

(51) Int. Cl. H10K 85/60; C07D 307/92; C07D 307/93; C07D 333/76; C07D 493/04; C07D 495/04; C09K 11/02; H10K 50/12

(52) U.S. Cl. CPC H10K 85/626; C07D 307/92; C07D 307/93; C07D 333/76; C07D 493/04; C07D 495/04; C09K 11/02; H10K 85/657; H10K 85/6574; H10K 85/6576; H10K 50/12

(57) ABSTRACT

Disclosed are an organic compound represented by Chemical Formula 1:

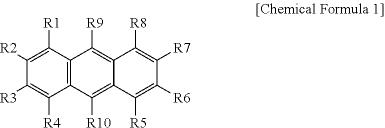

wherein one or more of R1 to R10 are bonded to L1 in Chemical Formula 1-A below, and one or more of R1 to R10 not bonded to L1 in Chemical Formula 1-A above are bonded to L2 in Chemical Formula 1-B below:

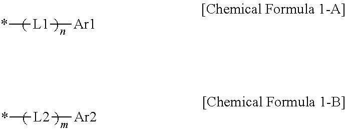

Ar1 is the following Chemical Formula 1-C,

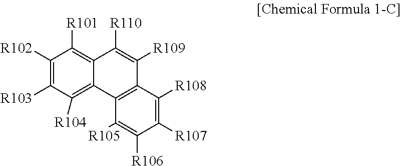

Ar2 is the following Chemical Formula 1-D,

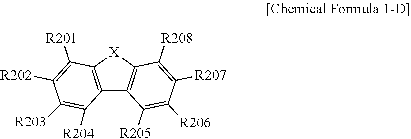

X is O or S, and the other substituents are as described in the specification; and an organic light emitting device including the same.

### CROSS-REFERENCE TO RELATED APPLICATIONS

This application claims priority to and the benefit of Korean Patent Application No. 10-2024-0035510 filed in the Korean Intellectual Property Office on Mar. 14, 2024, the entire contents of which are incorporated herein by reference.

### TECHNICAL FIELD

The present specification relates to an organic compound and an organic light emitting device including the same.

### BACKGROUND ART

An organic light emission phenomenon generally refers to a phenomenon converting electrical energy to light energy using an organic material. An organic light emitting device using the organic light emitting phenomenon usually has a structure including an anode, a cathode, and an organic material layer interposed therebetween. Here, the organic material layer has in many cases a multi-layered structure composed of different materials in order to improve the efficiency and stability of the organic light emitting device, and for example, may be composed of a hole injection layer, a hole transport layer, a light emitting layer, an electron transport layer, an electron injection layer, and the like. In the structure of the organic light emitting device, if a voltage is applied between two electrodes, holes are injected from an anode into the organic material layer and electrons are injected from a cathode into the organic material layer, and when the injected holes and electrons meet each other, an exciton is formed, and light is emitted when the exciton falls down again to a ground state.

There is a continuous need for developing a new material for the aforementioned organic light emitting device.

### RELATED ART DOCUMENTS

### Patent Document

Chinese Patent Publication No. 114621274

### SUMMARY OF THE INVENTION

The present invention has been made in an effort to provide an organic compound and an organic light emitting device including the same.

An exemplary embodiment of the present specification provides an organic compound of the following Chemical Formula 1.

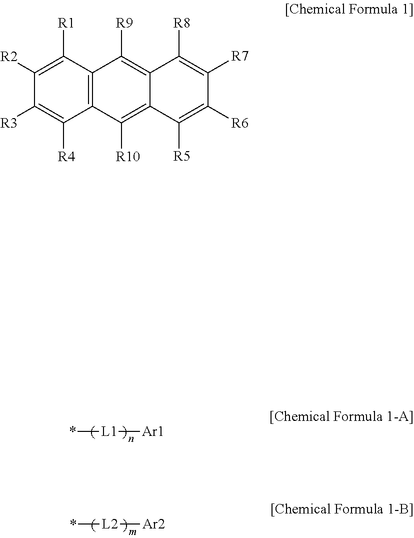

In Chemical Formula 1,
one or more of R1 to R10 are bonded to L1 in Chemical Formula 1-A above;
one or more of R1 to R10 not bonded to L1 in Chemical Formula 1-A above are bonded to L2 in Chemical Formula 1-B above,
R1 to R10, which are not bonded to L1 in Chemical Formula 1-A above and are not bonded to L2 in Chemical Formula 1-B above, are the same as or different from each other, and are each independently hydrogen; deuterium; a substituted or unsubstituted alkyl group; a substituted or unsubstituted cycloalkyl group; a substituted or unsubstituted aryl group; or a substituted or unsubstituted heteroaryl group,
L1 and L2 are the same as or different from each other, and are each independently a direct bond; a substituted or unsubstituted arylene group; or a substituted or unsubstituted heteroarylene group,
n and m are each an integer from 1 to 4,
Ar1 is the following Chemical Formula 1-C,

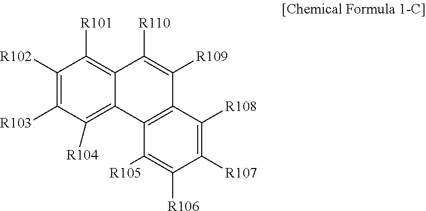

in Chemical Formula 1-C,
R101 to R110 are the same as or different from each other, and are each independently hydrogen; deuterium; a substituted or unsubstituted alkyl group; a substituted or unsubstituted cycloalkyl group; a substituted or unsubstituted aryl group; or a substituted or unsubstituted heteroaryl group, and one of R101 to R108 is bonded to L1,
Ar2 is the following Chemical Formula 1-D,

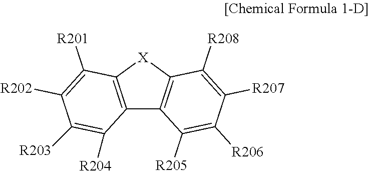

in Chemical Formula 1-D,
X is O or S,
R201 to R208 are the same as or different from each other, and are each independently hydrogen; deuterium; a substituted or unsubstituted alkyl group; a substituted or unsubstituted cycloalkyl group; a substituted or unsubstituted aryl group; or a substituted or unsubstituted heteroaryl group, and at least one pair adjacent to each other in R201 to R208 is bonded to each other to necessarily form one or more substituted or unsubstituted ring groups,
the substituted or unsubstituted ring group is a substituted or unsubstituted aromatic hydrocarbon ring group; a substituted or unsubstituted benzofuran ring group; or a substituted or unsubstituted benzothiophene ring group,
the substituted or unsubstituted ring group; or any one of R201 to R208 not forming the substituted or unsubstituted ring group is bonded to L2,
provided that when adjacent R201 to R208 are bonded to each other to form a substituted or unsubstituted aromatic hydrocarbon ring group, any one of R101, R103 to R106, and R108 in Chemical Formula 1-C is bonded to L1, or any one of R101 to R108, which are not bonded to L1 in Chemical Formula 1-C, and R201 to R208, which are not bonded to L2 in Chemical Formula 1-D, is necessarily one or more of a substituted or unsubstituted alkyl group, a substituted or unsubstituted cycloalkyl group, or a substituted or unsubstituted aryl group, or the substituted or unsubstituted ring group, which is not bonded to L2 in Chemical Formula 1-D, is substituted with a substituted or unsubstituted alkyl group, a substituted or unsubstituted cycloalkyl group, or a substituted or unsubstituted aryl group, and
when adjacent R201 to R208 are bonded to each other to form a substituted or unsubstituted benzofuran ring group or a substituted or unsubstituted benzothiophene ring group, R2, R3, R6, and R7 in Chemical Formula 1 are the same as or different from each other, and are each independently hydrogen or deuterium, and
* is a moiety bonded to Chemical Formula 1.

Another exemplary embodiment provides an organic light emitting device including: an anode; a cathode; and an organic material layer having one or more layers provided between the anode and the cathode, in which one or more layers of the organic material layers include the above-described organic compound.

The organic compound described in the present specification can be used as a material for an organic material layer of an organic light emitting device.

The organic compound according to at least one exemplary embodiment of the present specification may improve the efficiency, achieve low driving voltage and/or improve service life characteristics in the organic light emitting device.

### BRIEF DESCRIPTION OF THE DRAWINGS

FIG. 1 illustrates an example of an organic light emitting device in which a substrate 1, an anode 2, a light emitting layer 6, and a cathode 9 are sequentially stacked.

FIG. 2 illustrates an example of an organic light emitting device in which a substrate 1, an anode 2, a hole injection layer 3, a hole transport layer 4, a hole adjusting layer 5, a light emitting layer 6, an electron adjusting layer 7, an electron injection and transport layer 8, and a cathode 9 are sequentially stacked.

FIG. 3 is an MS graph of Compound A.

### DETAILED DESCRIPTION

Hereinafter, the present specification will be described in more detail.

When one part “includes” one constituent element in the present specification, unless otherwise specifically described, this does not mean that another constituent element is excluded, but means that another constituent element may be further included.

When one member is disposed “on” another member in the present specification, this includes not only a case where the one member is brought into contact with another member, but also a case where still another member is present between the two members.

In the present specification, “*” means a position bonded to a formula or a compound.

Examples of the substituents in the present specification will be described below, but are not limited thereto.

The term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

In the present specification, the term “substituted or unsubstituted” means being substituted with one or two or more substituents selected from the group consisting of deuterium; a halogen group; a nitrile group (—CN); a silyl group; an alkyl group; a cycloalkyl group; an alkoxy group; an aryloxy group; an alkylthioxy group; an arylthioxy group; an alkylsulfoxy group; an arylsulfoxy group; an alkenyl group; an amine group; an aryl group; or a heterocyclic group, being substituted with a substituent to which two or more substituents among the exemplified substituents are linked, or having no substituent. For example, “the substituent to which two or more substituents are linked” may be a biphenyl group. That is, the biphenyl group may also be an aryl group, and may be interpreted as a substituent to which two phenyl groups are linked.

In the present specification, the term “substituted or unsubstituted” means being substituted with one or two or more substituents selected from the group consisting of deuterium; a halogen group; a nitrile group; a nitro group; a hydroxyl group; an amino group; an alkoxy group; an aryloxy group; an alkyl group; a cycloalkyl group; an alkenyl group; an alkynyl group; an aryl group; and a heterocyclic group, being substituted with a substituent to which two or more substituents among the exemplified substituents are linked, or having no substituent.

In the present specification, the term “substituted or unsubstituted” means being substituted with one or two or more substituents selected from the group consisting of deuterium; a halogen group; a nitrile group; an alkyl group; an aryl group; and a heterocyclic group, being substituted with a substituent to which two or more substituents among the exemplified substituents are linked, or having no substituent.

Examples of the substituents will be described below; however, the substituents are not limited thereto.

In the present specification, examples of a halogen group include fluorine (—F), chlorine (—Cl), bromine (—Br) or iodine (—I).

In the present specification, a silyl group may be represented by a formula of —SiYaYbYc, and Ya, Yb, and Yc may be each hydrogen; a substituted or unsubstituted alkyl group; or a substituted or unsubstituted aryl group. Specific examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but are not limited thereto.

In the present specification, the alkyl group may be straight-chained or branched, and the number of carbon atoms thereof is not particularly limited, but is preferably 1 to 60. According to an exemplary embodiment, the number of carbon atoms of the alkyl group is 1 to 30. According to another exemplary embodiment, the number of carbon atoms of the alkyl group is 1 to 20. According to still another exemplary embodiment, the number of carbon atoms of the alkyl group is 1 to 10. Specific examples of the alkyl group include a methyl group, an ethyl group, a propyl group, an n-propyl group, an isopropyl group, a butyl group, an n-butyl group, an isobutyl group, a tert-butyl group, a pentyl group, an n-pentyl group, a hexyl group, an n-hexyl group, a heptyl group, an n-heptyl group, an octyl group, an n-octyl group, and the like, but are not limited thereto.

In the present specification, the alkoxy group may be straight-chained, branched, or cyclic. The number of carbon atoms of the alkoxy group is not particularly limited, but is preferably 1 to 20. Specific examples thereof include methoxy, ethoxy, n-propoxy, isopropoxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, and the like, but are not limited thereto.

Substituents including an alkyl group, an alkoxy group, and other alkyl group moieties described in the present specification include both a straight-chained form and a branched form.

In the present specification, an alkenyl group may be straight-chained or branched, and the number of carbon atoms thereof is not particularly limited, but is preferably 2 to 40. According to an exemplary embodiment, the number of carbon atoms of the alkenyl group is 2 to 20. According to another exemplary embodiment, the number of carbon atoms of the alkenyl group is 2 to 10. According to still another exemplary embodiment, the number of carbon atoms of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present specification, the alkynyl group may be straight-chained or branched as a substituent including a triple bond between a carbon atom and a carbon atom, and the number of carbon atoms thereof is not particularly limited, but is preferably 2 to 40. According to an exemplary embodiment, the number of carbon atoms of the alkynyl group is 2 to 20. According to another exemplary embodiment, the number of carbon atoms of the alkynyl group is 2 to 10.

In the present specification, a cycloalkyl group is not particularly limited, but has preferably 3 to 60 carbon atoms, and according to an exemplary embodiment, the number of carbon atoms of the cycloalkyl group is 3 to 30. According to another exemplary embodiment, the number of carbon atoms of the cycloalkyl group is 3 to 20. According to another embodiment, the number of carbon atoms of the cycloalkyl group is from 3 to 6. Specific examples thereof may include a cyclopropyl group, a cyclobutyl group, a cyclopentyl group, a cyclohexyl group, a cycloheptyl group, a cyclooctyl group and the like, but are not limited thereto.

In the present specification, an amine group is —NH₂, and the amine group may be substituted with the above-described alkyl group, aryl group, heterocyclic group, alkenyl group, cycloalkyl group, a combination thereof, and the like. The number of carbon atoms of the substituted amine group is not particularly limited, but is preferably 1 to 30. According to an exemplary embodiment, the number of carbon atoms of the amine group is 1 to 20. According to an exemplary embodiment, the number of carbon atoms of the amine group is 1 to 10. Specific examples of the substituted amine group include a methylamine group, a dimethylamine group, an ethylamine group, a diethylamine group, a phenylamine group, a 9,9-dimethylfluorenylphenylamine group, a pyridylphenylamine group, a diphenylamine group, a phenylpyridylamine group, a naphthylamine group, a biphenylamine group, an anthracenylamine group, a dibenzofuranylphenylamine group, a 9-methylanthracenylamine group, a diphenylamine group, a phenylnaphthylamine group, a ditolylamine group, a phenyltolylamine group, a diphenylamine group, and the like, but are not limited thereto.

In the present specification, an aryl group is not particularly limited, but has preferably 6 to 60 carbon atoms, and may be a monocyclic aryl group or a polycyclic aryl group. According to an exemplary embodiment, the number of carbon atoms of the aryl group is from 6 to 30. According to an exemplary embodiment, the number of carbon atoms of the aryl group is from 6 to 20. Examples of the monocyclic aryl group include a phenyl group, a biphenyl group, a terphenyl group, a quaterphenyl group, and the like, but are not limited thereto. Examples of the polycyclic aryl group include a naphthyl group, an anthracenyl group, a phenanthrene group, a pyrenyl group, a perylenyl group, a triphenylene group, a chrysenyl group, a fluorenyl group, a triphenylene group, and the like, but are not limited thereto.

In the present specification, the above-described description on the aryl group may be applied to an aryl group in an aryloxy group and an arylamine group.

In the present specification, the above-described description on the alkyl group may be applied to an alkyl group in the alkylthioxy group and the alkylsulfoxy group.

In the present specification, the above-described description on the aryl group may be applied to an aryl group in the arylthioxy group and the arylsulfoxy group.

In the present specification, a heterocyclic group is a cyclic group including one or more of N, O, P, S, Si, and Se as a heteroatom, and the number of carbon atoms thereof is not particularly limited, but is preferably 2 to 60. According to one embodiment, the number of carbon atoms of the heterocyclic group is from 2 to 30. According to one embodiment, the number of carbon atoms of the heterocyclic group is from 2 to 20. Examples of the heterocyclic group include a pyridine group, a pyrrole group, a pyrimidine group, a quinoline group, a pyridazinyl group, a furan group, a thiophene group, an imidazole group, a pyrazole group, a dibenzofuran group, a dibenzothiophene group, a carbazole group, a benzocarbazole group, a naphthobenzofuran group, a benzonaphthothiophene group, an indenocarbazole group, a triazinyl group, and the like, but are not limited thereto.

In the present specification, the above-described description on the heterocyclic group may be applied to a heteroaryl group except for an aromatic heteroaryl group.

In the present specification, the description on the aryl group may be applied to an arylene group except for a divalent arylene group.

In the present specification, the description on the heteroaryl group may be applied to a heteroarylene group except for a divalent heteroarylene group.

In the present specification, in a substituted or unsubstituted ring formed by bonding adjacent groups, the “ring” means a hydrocarbon ring; or a hetero ring.

For example, when a group is bonded to an adjacent group to form a ring, it is possible to form a substituted or unsubstituted aliphatic hydrocarbon ring; a substituted or unsubstituted aromatic hydrocarbon ring; a substituted or unsubstituted aliphatic hetero ring; a substituted or unsubstituted aromatic hetero ring; or a fused ring thereof. The hydrocarbon ring means a ring composed only of carbon and hydrogen atoms. The hetero ring means a ring including one or more selected from elements such as N, O, P, S, Si and Se. In the present specification, the aliphatic hydrocarbon ring, the aromatic hydrocarbon ring, the aliphatic hetero ring, and the aromatic hetero ring may be monocyclic or polycyclic.

In the present specification, the aliphatic hydrocarbon ring means a ring composed only of carbon and hydrogen atoms as a non-aromatic ring. Examples of the aliphatic hydrocarbon ring include cyclopropane, cyclobutane, cyclobutene, cyclopentane, cyclopentene, cyclohexane, cyclohexene, 1,4-cyclohexadiene, cycloheptane, cycloheptene, cyclooctane, cyclooctene, and the like, but are not limited thereto.

In the present specification, an aromatic hydrocarbon ring means an aromatic ring composed only of carbon and hydrogen atoms. Examples of the aromatic hydrocarbon ring include benzene, naphthalene, anthracene, phenanthrene, perylene, fluoranthene, triphenylene, phenalene, pyrene, tetracene, chrysene, pentacene, fluorene, indene, acenaphthylene, benzofluorene, spirofluorene, and the like, but are not limited thereto. In the present specification, the aromatic hydrocarbon ring may be interpreted to have the same meaning as the aryl group.

In the present specification, an aliphatic hetero ring means an aliphatic ring including one or more of hetero atoms. Examples of the aliphatic hetero ring include oxirane, tetrahydrofuran, 1,4-dioxane, pyrrolidine, piperidine, morpholine, oxepane, azocane, thiocane, and the like, but are not limited thereto.

In the present specification, an aromatic hetero ring means an aromatic ring including one or more of hetero atoms. Examples of the aromatic hetero ring include pyridine, pyrrole, pyrimidine, pyridazine, furan, thiophene, imidazole, pyrazole, oxazole, isoxazole, thiazole, isothiazole, triazole, oxadiazole, thiadiazole, dithiazole, tetrazole, pyran, thiopyran, diazine, oxazine, thiazine, dioxine, triazine, tetrazine, isoquinoline, quinoline, quinone, quinazoline, quinoxaline, naphthyridine, acridine, phenanthridine, diaza naphthalene, triazaindene, indole, indolizine, benzothiazole, benzoxazole, benzoimidazole, benzothiophene, benzofuran, dibenzothiophene, dibenzofuran, carbazole, benzocarbazole, dibenzocarbazole, phenazine, imidazopyridine, phenoxazine, indolocarbazole, indenocarbazole, and the like, but are not limited thereto.

Hereinafter, preferred exemplary embodiments of the present invention will be described in detail. However, the exemplary embodiments of the present invention may be modified into various other forms, and the scope of the present invention is not limited to the exemplary embodiments which will be described below.

Hereinafter, an organic compound of the following Chemical Formula 1 will be described in detail.

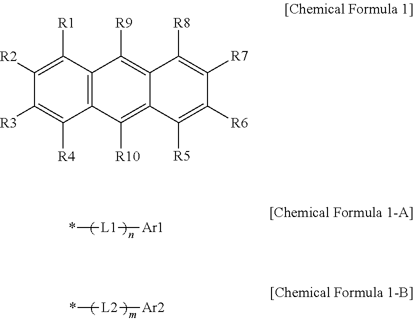

In Chemical Formula 1,
one or more of R1 to R10 are bonded to L1 in Chemical Formula 1-A above;
one or more of R1 to R10 not bonded to L1 in Chemical Formula 1-A above are bonded to L2 in Chemical Formula 1-B above,
R1 to R10, which are not bonded to L1 in Chemical Formula 1-A above and are not bonded to L2 in Chemical Formula 1-B above, are the same as or different from each other, and are each independently hydrogen; deuterium; a substituted or unsubstituted alkyl group; a substituted or unsubstituted cycloalkyl group; a substituted or unsubstituted aryl group; or a substituted or unsubstituted heteroaryl group,
L1 and L2 are the same as or different from each other, and are each independently a direct bond; a substituted or unsubstituted arylene group; or a substituted or unsubstituted heteroarylene group,
n and m are each an integer from 1 to 4,
Ar1 is the following Chemical Formula 1-C,

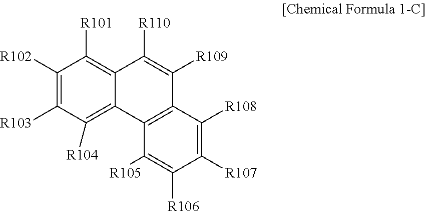

in Chemical Formula 1-C,
R101 to R110 are the same as or different from each other, and are each independently hydrogen; deuterium; a substituted or unsubstituted alkyl group; a substituted or unsubstituted cycloalkyl group; a substituted or unsubstituted aryl group; or a substituted or unsubstituted heteroaryl group, and one of R101 to R108 is bonded to L1,
Ar2 is the following Chemical Formula 1-D,

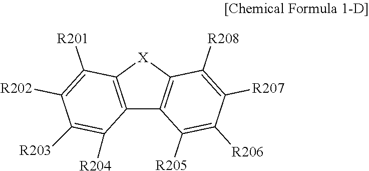

in Chemical Formula 1-D,
X is O or S,
R201 to R208 are the same as or different from each other, and are each independently hydrogen; deuterium; a substituted or unsubstituted alkyl group; a substituted or unsubstituted cycloalkyl group; a substituted or unsubstituted aryl group; or a substituted or unsubstituted heteroaryl group, and at least one pair adjacent to each other in R201 to R208 is bonded to each other to necessarily form one or more substituted or unsubstituted ring groups,
the substituted or unsubstituted ring group is a substituted or unsubstituted aromatic hydrocarbon ring group; a substituted or unsubstituted benzofuran ring group; or a substituted or unsubstituted benzothiophene ring group,
the substituted or unsubstituted ring group; or any one of R201 to R208 not forming the substituted or unsubstituted ring group is bonded to L2,
provided that when adjacent R201 to R208 are bonded to each other to form a substituted or unsubstituted aromatic hydrocarbon ring group, any one of R101, R103 to R106, and R108 in Chemical Formula 1-C is bonded to L1, or any one of R101 to R108, which are not bonded to L1 in Chemical Formula 1-C, and R201 to R208, which are not bonded to L2 in Chemical Formula 1-D, is necessarily one or more of a substituted or unsubstituted alkyl group, a substituted or unsubstituted cycloalkyl group, or a substituted or unsubstituted aryl group, or the substituted or unsubstituted ring group, which is not bonded to L2 in Chemical Formula 1-D, is substituted with a substituted or unsubstituted alkyl group, a substituted or unsubstituted cycloalkyl group, or a substituted or unsubstituted aryl group, and
when adjacent R201 to R208 are bonded to each other to form a substituted or unsubstituted benzofuran ring group or a substituted or unsubstituted benzothiophene ring group, R2, R3, R6, and R7 in Chemical Formula 1 are the same as or different from each other, and are each independently hydrogen or deuterium, and
* is a moiety bonded to Chemical Formula 1.

In an exemplary embodiment of the present specification, the substituted or unsubstituted ring groups of Chemical Formula 1-D are organic compounds represented by the following 1-D-1 to 1-D-9.

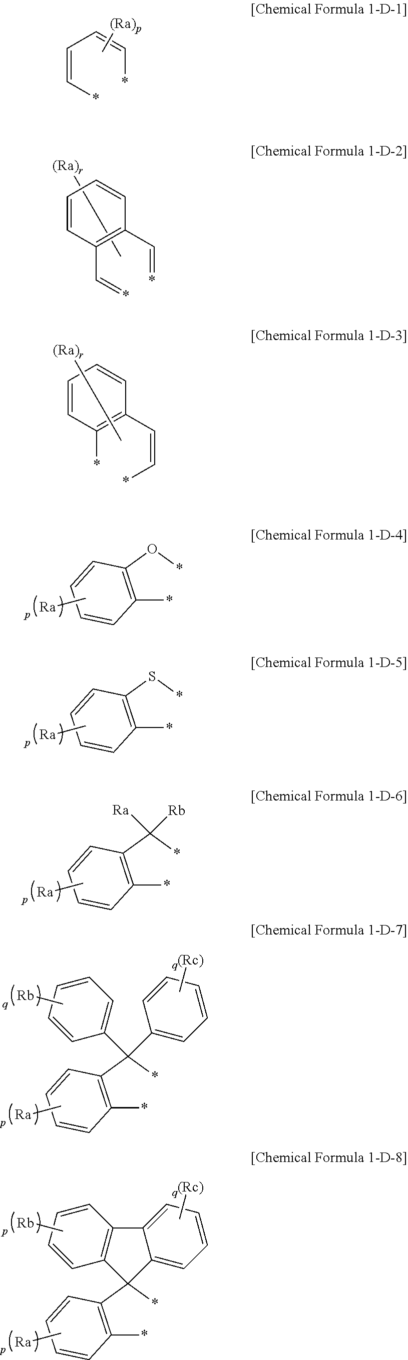

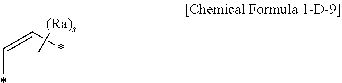

In Chemical Formulae 1-D-1 to 1-D-9,
Ra to Rc are the same as or different from each other, and are each independently hydrogen; deuterium; a substituted or unsubstituted alkyl group; a substituted or unsubstituted aryl group; or a substituted or unsubstituted heterocyclic group, or optionally bonded to an adjacent group to form a substituted or unsubstituted ring group, or bonded to L2 above,
p is an integer from 1 to 4, and when p is 2 or higher, two or more Ra's, Rb's, or Rc's are the same as or different from each other,
q is an integer from 1 to 5, and when q is 2 or higher, two or more Rb's or Rc's are the same as or different from each other,
r is an integer from 1 to 6, and when r is 2 or higher, two or more Ra's are the same as or different from each other,
s is 1 or 2, and when s is 2, two Ra's are the same as or different from each other, and
* is a moiety bonded to an adjacent pair of R201 to R208 in Chemical Formula 1-D.

In an exemplary embodiment of the present specification, p is an integer from 1 to 4.

In an exemplary embodiment of the present specification, p is 4.

In an exemplary embodiment of the present specification, p is 3.

In an exemplary embodiment of the present specification, p is 2.

In an exemplary embodiment of the present specification, p is 1.

In an exemplary embodiment of the present specification, q is an integer from 1 to 5.

In an exemplary embodiment of the present specification, q is 5.

In an exemplary embodiment of the present specification, q is 4.

In an exemplary embodiment of the present specification, q is 3.

In an exemplary embodiment of the present specification, q is 2.

In an exemplary embodiment of the present specification, q is 1.

In an exemplary embodiment of the present specification, r is an integer from 1 to 6.

In an exemplary embodiment of the present specification, r is 6.

In an exemplary embodiment of the present specification, r is 5.

In an exemplary embodiment of the present specification, r is 4.

In an exemplary embodiment of the present specification, r is 3.

In an exemplary embodiment of the present specification, r is 2.

In an exemplary embodiment of the present specification, r is 1.

In an exemplary embodiment of the present specification, s is an integer of 1 or 6.

In an exemplary embodiment of the present specification, s is 2.

In an exemplary embodiment of the present specification, s is 1.

In an exemplary embodiment of the present specification, Ra to Rc are the same as or different from each other, and are each independently hydrogen; deuterium; a substituted or unsubstituted alkyl group having 1 to 30 carbon atoms; a substituted or unsubstituted aryl group having 6 to 30 carbon atoms; or a substituted or unsubstituted heterocyclic group having 2 to 30 carbon atoms, or optionally bonded to an adjacent group to form a substituted or unsubstituted ring group.

In an exemplary embodiment of the present specification, Ra to Rc are the same as or different from each other, and are each independently hydrogen; deuterium; a substituted or unsubstituted alkyl group having 1 to 20 carbon atoms; a substituted or unsubstituted aryl group having 6 to 20 carbon atoms; or a substituted or unsubstituted heterocyclic group having 2 to 20 carbon atoms, or optionally bonded to an adjacent group to form a substituted or unsubstituted ring group.

In an exemplary embodiment of the present specification, Ra to Rc are the same as or different from each other, and are each independently hydrogen; deuterium; a substituted or unsubstituted alkyl group having 1 to 10 carbon atoms; a substituted or unsubstituted aryl group having 6 to 10 carbon atoms; or a substituted or unsubstituted heterocyclic group having 2 to 10 carbon atoms, or optionally bonded to an adjacent group to form a substituted or unsubstituted ring group.

In an exemplary embodiment of the present specification, Ra to Rc are the same as or different from each other, and are each independently hydrogen or deuterium, or optionally bonded to an adjacent group to form a substituted or unsubstituted ring group.

In an exemplary embodiment of the present specification, Ra to Rc are the same as or different from each other, and are each independently hydrogen, or optionally bonded to an adjacent group to form a substituted or unsubstituted ring group.

In an exemplary embodiment of the present specification, Ra to Rc are the same as or different from each other, and are each independently deuterium, or optionally bonded to an adjacent group to form a substituted or unsubstituted ring group.

An exemplary embodiment of the present specification is an organic compound in which Chemical Formula 1 above is any one of the following Chemical Formulae 1-1 to 1-4.

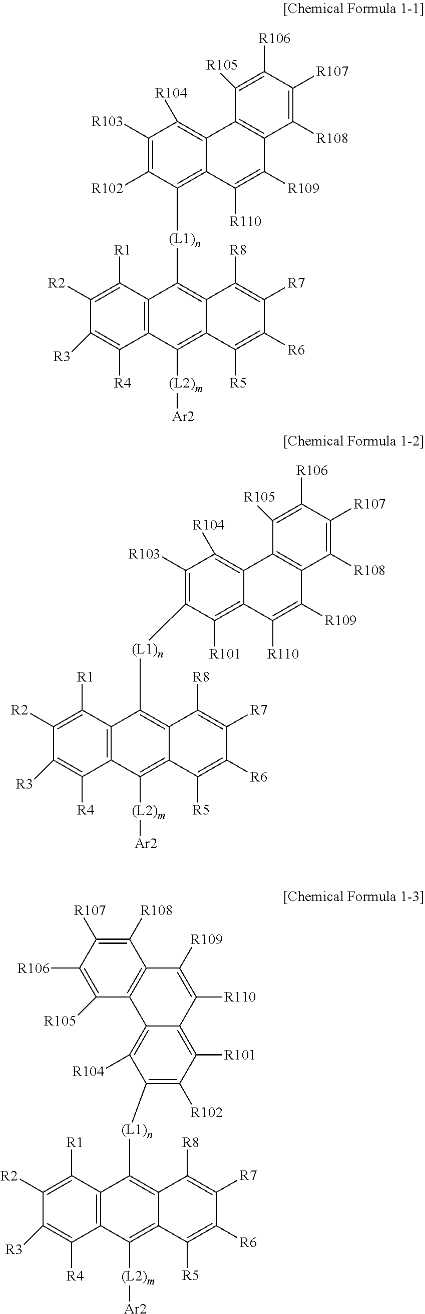

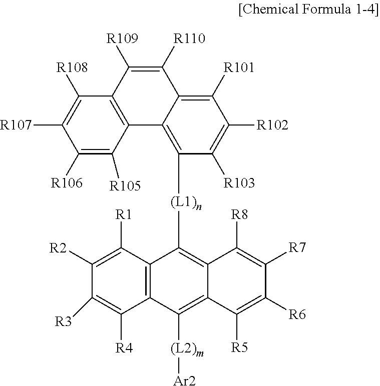

In Chemical Formula 1-1 to 1-4,
the definitions of L1, L2, Ar2, R101 to R110, n, and m are the same as the definitions in Chemical Formula 1 above, and
R1 to R8 are hydrogen; deuterium; a substituted or unsubstituted alkyl group; a substituted or unsubstituted cycloalkyl group; a substituted or unsubstituted aryl group; or a substituted or unsubstituted heteroaryl group.

In the present specification, Chemical Formula 1 above is Chemical Formula 1-1 above.

In the present specification, Chemical Formula 1 above is Chemical Formula 1-2 above.

In the present specification, Chemical Formula 1 above is Chemical Formula 1-3 above.

In the present specification, Chemical Formula 1 above is Chemical Formula 1-4 above.

An exemplary embodiment of the present specification is an organic compound in which Chemical Formula 1 above is any one of the following Chemical Formulae 1-5 to 1-8.

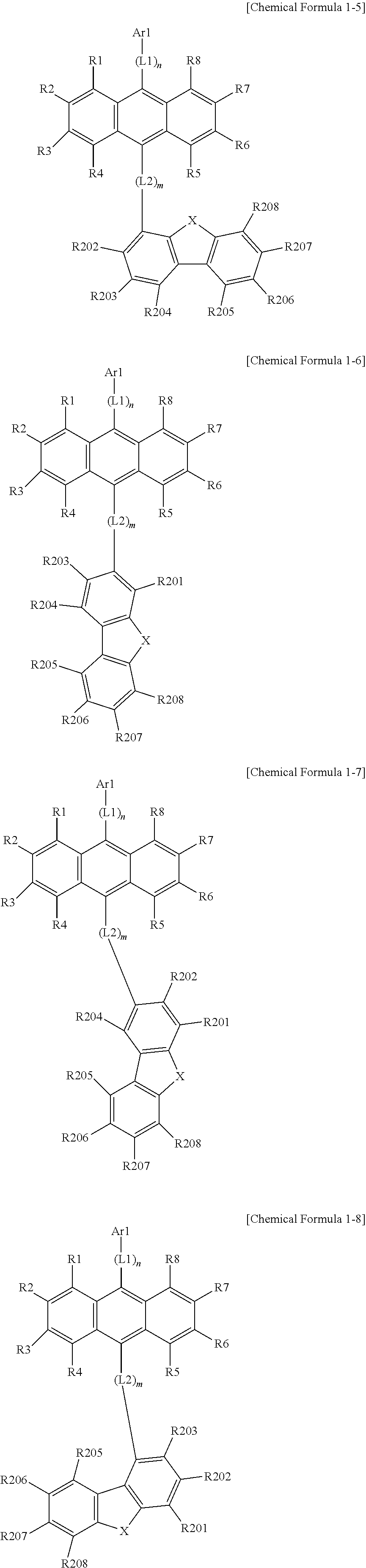

In Chemical Formulae 1-5 to 1-8,
the definitions of L1, L2, Ar1, X, R201 to R208, n, and m are the same as the definitions in Chemical Formula 1 above, and
R1 to R8 are hydrogen; deuterium; a substituted or unsubstituted alkyl group; a substituted or unsubstituted cycloalkyl group; a substituted or unsubstituted aryl group; or a substituted or unsubstituted heteroaryl group.

In the present specification, Chemical Formula 1 above is Chemical Formula 1-5 above.

In the present specification, Chemical Formula 1 above is Chemical Formula 1-6 above.

In the present specification, Chemical Formula 1 above is Chemical Formula 1-7 above.

In the present specification, Chemical Formula 1 above is Chemical Formula 1-8 above.

In an exemplary embodiment of the present specification, R1 to R10, which are not bonded to L1 in Chemical Formula 1-A above and are not bonded to L2 in Chemical Formula 1-B, are the same as or different from each other, and are each independently hydrogen; deuterium; a substituted or unsubstituted alkyl group, a substituted or unsubstituted cycloalkyl group, a substituted or unsubstituted aryl group, or a substituted or unsubstituted heteroaryl group.

In an exemplary embodiment of the present specification, R1 to R10, which are not bonded to L1 in Chemical Formula 1-A above and are not bonded to L2 in Chemical Formula 1-B, are the same as or different from each other, and are each independently hydrogen; deuterium; a substituted or unsubstituted alkyl group having 1 to 30 carbon atoms, a substituted or unsubstituted cycloalkyl group having 3 to 60 carbon atoms, a substituted or unsubstituted aryl group having 6 to 60 carbon atoms, or a substituted or unsubstituted heteroaryl group having 2 to 60 carbon atoms.

In an exemplary embodiment of the present specification, R1 to R10, which are not bonded to L1 in Chemical Formula 1-A above and are not bonded to L2 in Chemical Formula 1-B, are the same as or different from each other, and are each independently hydrogen; deuterium; a substituted or unsubstituted alkyl group having 1 to 10 carbon atoms, a substituted or unsubstituted cycloalkyl group having 3 to 30 carbon atoms, a substituted or unsubstituted aryl group having 6 to 30 carbon atoms, or a substituted or unsubstituted heteroaryl group having 2 to 30 carbon atoms.

In an exemplary embodiment of the present specification, R1 to R10, which are not bonded to L1 in Chemical Formula 1-A above and are not bonded to L2 in Chemical Formula 1-B, are the same as or different from each other, and are each independently hydrogen; deuterium; a substituted or unsubstituted alkyl group having 1 to 10 carbon atoms, a substituted or unsubstituted cycloalkyl group having 3 to 20 carbon atoms, a substituted or unsubstituted aryl group having 6 to 20 carbon atoms, or a substituted or unsubstituted heteroaryl group having 2 to 20 carbon atoms.

In an exemplary embodiment of the present specification, R1 to R10, which are not bonded to L1 in Chemical Formula 1-A above and are not bonded to L2 in Chemical Formula 1-B, are the same as or different from each other, and are each independently hydrogen; deuterium; a substituted or unsubstituted alkyl group having 1 to 10 carbon atoms, a substituted or unsubstituted cycloalkyl group having 3 to 10 carbon atoms, a substituted or unsubstituted aryl group having 6 to 10 carbon atoms, or a substituted or unsubstituted heteroaryl group having 2 to 10 carbon atoms.

In an exemplary embodiment of the present specification, R1 to R10, which are not bonded to L1 in Chemical Formula 1-A above and are not bonded to L2 in Chemical Formula 1-B, are the same as or different from each other, and are each independently hydrogen; deuterium; a substituted or unsubstituted phenyl group; a substituted or unsubstituted biphenyl group; a substituted or unsubstituted terphenyl group; a substituted or unsubstituted quaterphenyl group; a substituted or unsubstituted naphthyl group; a substituted or unsubstituted phenanthrenyl group; a substituted or unsubstituted fluorenyl group; a substituted or unsubstituted spirobifluorenyl group; a substituted or unsubstituted triphenylenyl group; a substituted or unsubstituted furan group; a substituted or unsubstituted benzofuran group; a substituted or unsubstituted dibenzofuran group; a substituted or unsubstituted naphthobenzofuran group; a substituted or unsubstituted thiophene group; a substituted or unsubstituted benzothiophene group; a substituted or unsubstituted dibenzothiophene group; a substituted or unsubstituted naphthobenzothiophene group; a substituted or unsubstituted carbazole group; a substituted or unsubstituted pyridyl group; or a substituted or unsubstituted pyrimidyl group.

In an exemplary embodiment of the present specification, R1 to R10, which are not bonded to L1 in Chemical Formula 1-A above and are not bonded to L2 in Chemical Formula 1-B, are the same as or different from each other, and are each independently hydrogen or deuterium.

In an exemplary embodiment of the present specification, R1 to R10, which are not bonded to L1 in Chemical Formula 1-A above and are not bonded to L2 in Chemical Formula 1-B, are hydrogen.

In an exemplary embodiment of the present specification, R1 to R10, which are not bonded to L1 in Chemical Formula 1-A above and are not bonded to L2 in Chemical Formula 1-B, are deuterium.

In an exemplary embodiment of the present specification, L1 and L2 are the same as or different from each other, and are each independently a direct bond; a substituted or unsubstituted arylene group; or a substituted or unsubstituted heteroarylene group.

In an exemplary embodiment of the present specification, L1 and L2 are the same as or different from each other, and are each independently a direct bond; an arylene group unsubstituted or substituted with deuterium, an alkyl group having 1 to 10 carbon atoms, a cycloalkyl group having 3 to 30 carbon atoms, an aryl group having 6 to 60 carbon atoms, or a heteroaryl group having 2 to 60 carbon atoms; or a heteroarylene group unsubstituted or substituted with deuterium, an alkyl group having 1 to 10 carbon atoms, a cycloalkyl group having 3 to 30 carbon atoms, an aryl group having 6 to 60 carbon atoms, or a heteroaryl group having 2 to 60 carbon atoms.

In an exemplary embodiment of the present specification, L1 and L2 are the same as or different from each other, and are each independently a direct bond; an arylene group unsubstituted or substituted with deuterium, an alkyl group having 1 to 10 carbon atoms, a cycloalkyl group having 3 to 20 carbon atoms, an aryl group having 6 to 30 carbon atoms, or a heteroaryl group having 2 to 30 carbon atoms; or a heteroarylene group unsubstituted or substituted with deuterium, an alkyl group having 1 to 10 carbon atoms, a cycloalkyl group having 3 to 20 carbon atoms, an aryl group having 6 to 30 carbon atoms, or a heteroaryl group having 2 to 30 carbon atoms.

In an exemplary embodiment of the present specification, L1 and L2 are the same as or different from each other, and are each independently a direct bond; an arylene group unsubstituted or substituted with deuterium, an alkyl group having 1 to 10 carbon atoms, a cycloalkyl group having 3 to 10 carbon atoms, an aryl group having 6 to 20 carbon atoms, or a heteroaryl group having 2 to 20 carbon atoms; or a heteroarylene group unsubstituted or substituted with deuterium, an alkyl group having 1 to 10 carbon atoms, a cycloalkyl group having 3 to 10 carbon atoms, an aryl group having 6 to 20 carbon atoms, or a heteroaryl group having 2 to 20 carbon atoms.

In an exemplary embodiment of the present specification, L1 and L2 are the same as or different from each other, and are each independently a direct bond; an arylene group unsubstituted or substituted with deuterium, an alkyl group having 1 to 6 carbon atoms, a cycloalkyl group having 3 to 10 carbon atoms, an aryl group having 6 to 10 carbon atoms, or a heteroaryl group having 2 to 10 carbon atoms; or a heteroarylene group unsubstituted or substituted with deuterium, an alkyl group having 1 to 6 carbon atoms, a cycloalkyl group having 3 to 10 carbon atoms, an aryl group having 6 to 10 carbon atoms, or a heteroaryl group having 2 to 10 carbon atoms.

In an exemplary embodiment of the present specification, L1 and L2 are the same as or different from each other, and are each independently a direct bond; a substituted or unsubstituted naphthylene group; or a substituted or unsubstituted biphenylene group.

In an exemplary embodiment of the present specification, L1 and L2 are the same as or different from each other, and are each independently a direct bond; a naphthylene group unsubstituted or substituted with deuterium, an alkyl group having 1 to 10 carbon atoms, a cycloalkyl group having 3 to 30 carbon atoms, an aryl group having 6 to 60 carbon atoms, or a heteroaryl group having 2 to 60 carbon atoms; or a biphenylene group unsubstituted or substituted with deuterium, an alkyl group having 1 to 10 carbon atoms, a cycloalkyl group having 3 to 30 carbon atoms, an aryl group having 6 to 60 carbon atoms, or a heteroaryl group having 2 to 60 carbon atoms.

In an exemplary embodiment of the present specification, L1 and L2 are the same as or different from each other, and are each independently a direct bond; a naphthylene group unsubstituted or substituted with deuterium, an alkyl group having 1 to 10 carbon atoms, a cycloalkyl group having 3 to 20 carbon atoms, an aryl group having 6 to 30 carbon atoms, or a heteroaryl group having 2 to 30 carbon atoms; or a biphenylene group unsubstituted or substituted with deuterium, an alkyl group having 1 to 10 carbon atoms, a cycloalkyl group having 3 to 20 carbon atoms, an aryl group having 6 to 30 carbon atoms, or a heteroaryl group having 2 to 30 carbon atoms.

In an exemplary embodiment of the present specification, L1 and L2 are the same as or different from each other, and are each independently a direct bond; a naphthylene group unsubstituted or substituted with deuterium, an alkyl group having 1 to 10 carbon atoms, a cycloalkyl group having 3 to 10 carbon atoms, an aryl group having 6 to 20 carbon atoms, or a heteroaryl group having 2 to 20 carbon atoms; or a biphenylene group unsubstituted or substituted with deuterium, an alkyl group having 1 to 10 carbon atoms, a cycloalkyl group having 3 to 10 carbon atoms, an aryl group having 6 to 20 carbon atoms, or a heteroaryl group having 2 to 20 carbon atoms.

In an exemplary embodiment of the present specification, L1 and L2 are the same as or different from each other, and are each independently a direct bond; a naphthylene group unsubstituted or substituted with deuterium, an alkyl group having 1 to 6 carbon atoms, a cycloalkyl group having 3 to 10 carbon atoms, an aryl group having 6 to 10 carbon atoms, or a heteroaryl group having 2 to 10 carbon atoms; or a biphenylene group unsubstituted or substituted with deuterium, an alkyl group having 1 to 6 carbon atoms, a cycloalkyl group having 3 to 10 carbon atoms, an aryl group having 6 to 10 carbon atoms, or a heteroaryl group having 2 to 10 carbon atoms.

In an exemplary embodiment of the present specification, L1 and L2 are the same as or different from each other, and are each independently a direct bond; a substituted or unsubstituted phenylene group; a substituted or unsubstituted biphenylene group; a substituted or unsubstituted terphenylene group; a substituted or unsubstituted naphthylene group; or a substituted or unsubstituted fluorenylene group.

In an exemplary embodiment of the present specification, L1 and L2 are each a direct bond.

In an exemplary embodiment of the present specification, n is an integer from 1 to 4.

In an exemplary embodiment of the present specification, n is 4.

In an exemplary embodiment of the present specification, n is 3.

In an exemplary embodiment of the present specification, n is 2.

In an exemplary embodiment of the present specification, n is 1.

In an exemplary embodiment of the present specification, m is an integer from 1 to 4.

In an exemplary embodiment of the present specification, m is 4.

In an exemplary embodiment of the present specification, m is 3.

In an exemplary embodiment of the present specification, m is 2.

In an exemplary embodiment of the present specification, m is 1.

In an exemplary embodiment of the present specification, R101 to R110 are the same as or different from each other, and are each independently hydrogen; deuterium; a substituted or unsubstituted alkyl having 1 to 30 carbon atoms; a substituted or unsubstituted cycloalkyl group having 3 to 60 carbon atoms; a substituted or unsubstituted aryl group having 6 to 60 carbon atoms; or a substituted or unsubstituted heteroaryl group having 2 to 60 carbon atoms, and one of R101 to R108 is necessarily bonded to L1.

In an exemplary embodiment of the present specification, R101 to R110 are the same as or different from each other, and are each independently hydrogen; deuterium; a substituted or unsubstituted alkyl group having 1 to 10 carbon atoms; a substituted or unsubstituted cycloalkyl group having 3 to 30 carbon atoms; a substituted or unsubstituted aryl group having 6 to 30 carbon atoms; or a substituted or unsubstituted heteroaryl group having 2 to 30 carbon atoms, and one of R101 to R108 is necessarily bonded to L1.

In an exemplary embodiment of the present specification, R101 to R110 are the same as or different from each other, and are each independently hydrogen; deuterium; a substituted or unsubstituted alkyl group having 1 to 10 carbon atoms; a substituted or unsubstituted cycloalkyl group having 3 to 20 carbon atoms; a substituted or unsubstituted aryl group having 6 to 20 carbon atoms; or a substituted or unsubstituted heteroaryl group having 2 to 20 carbon atoms, and one of R101 to R108 is necessarily bonded to L1.

In an exemplary embodiment of the present specification, R101 to R110 are the same as or different from each other, and are each independently hydrogen; deuterium; a substituted or unsubstituted alkyl group having 1 to 10 carbon atoms; a substituted or unsubstituted cycloalkyl group having 3 to 20 carbon atoms; a substituted or unsubstituted aryl group having 6 to 10 carbon atoms; or a substituted or unsubstituted heteroaryl group having 2 to 10 carbon atoms, and one of R101 to R108 is necessarily bonded to L1.

In an exemplary embodiment of the present specification, R101 to R110 are the same as or different from each other, and are each independently hydrogen; deuterium; a substituted or unsubstituted methyl group; a substituted or unsubstituted ethyl group; a substituted or unsubstituted propyl group; a substituted or unsubstituted isopropyl group; a substituted or unsubstituted tert-butyl group; a substituted or unsubstituted tetrahydronaphthalene group; a substituted or unsubstituted phenyl group; a substituted or unsubstituted biphenyl group; a substituted or unsubstituted terphenyl group; a substituted or unsubstituted quaterphenyl group; a substituted or unsubstituted naphthyl group; a substituted or unsubstituted phenanthrenyl group; a substituted or unsubstituted fluorenyl group; a substituted or unsubstituted spirobifluorenyl group; a substituted or unsubstituted triphenylenyl group; a substituted or unsubstituted furan group; a substituted or unsubstituted benzofuran group; a substituted or unsubstituted dibenzofuran group; a substituted or unsubstituted naphthobenzofuran group; a substituted or unsubstituted thiophene group; a substituted or unsubstituted benzothiophene group; a substituted or unsubstituted dibenzothiophene group; a substituted or unsubstituted naphthobenzothiophene group; a substituted or unsubstituted carbazole group; a substituted or unsubstituted pyridyl group; or a substituted or unsubstituted pyrimidyl group, and one of R101 to R108 is necessarily bonded to L1.

X is O or S.

X is O.

X is S.

In an exemplary embodiment of the present specification, R201 to R208 are the same as or different from each other, and are each independently hydrogen; deuterium; a substituted or unsubstituted alkyl group; a substituted or unsubstituted cycloalkyl group; a substituted or unsubstituted aryl group; or a substituted or unsubstituted heteroaryl group, and at least one pair adjacent to each other in R201 to R208 is bonded to each other to necessarily form one or more substituted or unsubstituted ring groups.

In an exemplary embodiment of the present specification, R201 to R208 are the same as or different from each other, and are each independently hydrogen; deuterium; a substituted or unsubstituted alkyl group having 1 to 30 carbon atoms, a substituted or unsubstituted cycloalkyl group having 3 to 60 carbon atoms, a substituted or unsubstituted aryl group having 6 to 60 carbon atoms, or a substituted or unsubstituted heteroaryl group having 2 to 60 carbon atoms, and at least one pair adjacent to each other in R201 to R208 is bonded to each other to necessarily form one or more substituted or unsubstituted ring groups.

In an exemplary embodiment of the present specification, R201 to R208 are the same as or different from each other, and are each independently hydrogen; deuterium; a substituted or unsubstituted alkyl group having 1 to 10 carbon atoms, a substituted or unsubstituted cycloalkyl group having 3 to 30 carbon atoms, a substituted or unsubstituted aryl group having 6 to 30 carbon atoms, or a substituted or unsubstituted heteroaryl group having 2 to 30 carbon atoms, and at least one pair adjacent to each other in R201 to R208 is bonded to each other to necessarily form one or more substituted or unsubstituted ring groups.

In an exemplary embodiment of the present specification, R201 to R208 are the same as or different from each other, and are each independently hydrogen; deuterium; a substituted or unsubstituted alkyl group having 1 to 10 carbon atoms, a substituted or unsubstituted cycloalkyl group having 3 to 20 carbon atoms, a substituted or unsubstituted aryl group having 6 to 20 carbon atoms, or a substituted or unsubstituted heteroaryl group having 2 to 20 carbon atoms, and at least one pair adjacent to each other in R201 to R208 is bonded to each other to necessarily form one or more substituted or unsubstituted ring groups.

In an exemplary embodiment of the present specification, R201 to R208 are the same as or different from each other, and are each independently hydrogen; deuterium; a substituted or unsubstituted alkyl group having 1 to 10 carbon atoms, a substituted or unsubstituted cycloalkyl group having 3 to 10 carbon atoms, a substituted or unsubstituted aryl group having 6 to 10 carbon atoms, or a substituted or unsubstituted heteroaryl group having 2 to 10 carbon atoms, and at least one pair adjacent to each other in R201 to R208 is bonded to each other to necessarily form one or more substituted or unsubstituted ring groups.

In an exemplary embodiment of the present specification, R201 to R208 are the same as or different from each other, and are each independently hydrogen; deuterium; a substituted or unsubstituted methyl group; a substituted or unsubstituted ethyl group; a substituted or unsubstituted propyl group; a substituted or unsubstituted isopropyl group; a substituted or unsubstituted tert-butyl group; a substituted or unsubstituted phenyl group; a substituted or unsubstituted biphenyl group; a substituted or unsubstituted terphenyl group; a substituted or unsubstituted quaterphenyl group; a substituted or unsubstituted naphthyl group; a substituted or unsubstituted phenanthrenyl group; a substituted or unsubstituted fluorenyl group; a substituted or unsubstituted spirobifluorenyl group; a substituted or unsubstituted triphenylenyl group; a substituted or unsubstituted furan group; a substituted or unsubstituted benzofuran group; a substituted or unsubstituted dibenzofuran group; a substituted or unsubstituted naphthobenzofuran group; a substituted or unsubstituted thiophene group; a substituted or unsubstituted benzothiophene group; a substituted or unsubstituted dibenzothiophene group; a substituted or unsubstituted naphthobenzothiophene group; a substituted or unsubstituted carbazole group; a substituted or unsubstituted pyridyl group; or a substituted or unsubstituted pyrimidyl group, and at least one pair adjacent to each other in R201 to R208 is bonded to each other to necessarily form one or more substituted or unsubstituted ring groups.

According to an exemplary embodiment of the present specification, the substituted or unsubstituted ring group; or any one of R201 to R208 not forming the substituted or unsubstituted ring group is bonded to L2.

According to an exemplary embodiment of the present specification, R201 to R208, which are not bonded to L2, are the same as or different from each other, and are each independently hydrogen; or deuterium.

According to an exemplary embodiment of the present specification, R201 to R208, which are not bonded to L2, are the same as or different from each other, and are each independently hydrogen.

According to an exemplary embodiment of the present specification, R201 to R208, which are not bonded to L2, are the same as or different from each other, and are each independently deuterium.

According to an exemplary embodiment of the present specification, the substituted or unsubstituted ring group is a substituted or unsubstituted aromatic hydrocarbon ring group having 4 to 30 carbon atoms; a substituted or unsubstituted benzofuran ring group; or a substituted or unsubstituted benzothiophene ring group.

According to an exemplary embodiment of the present specification, when adjacent R201 to R208 are bonded to each other to form a substituted or unsubstituted aromatic hydrocarbon ring group, any one of R101, R103 to R106, and R108 in Chemical Formula 1-C is bonded to L1, or any one of R101 to R108, which are not bonded to L1 in Chemical Formula 1-C, and R201 to R208, which are not bonded to L2 in Chemical Formula 1-D, is necessarily one or more of a substituted or unsubstituted alkyl group having 1 to 10 carbon atoms, a substituted or unsubstituted cycloalkyl group having 3 to 30 carbon atoms, or a substituted or unsubstituted aryl group having 6 to 30 carbon atoms, or the substituted or unsubstituted ring group, which is not bonded to L2 in Chemical Formula 1-D, is substituted with a substituted or unsubstituted alkyl group having 1 to 10 carbon atoms, a substituted or unsubstituted cycloalkyl group having 3 to 30 carbon atoms, or a substituted or unsubstituted aryl group having 6 to 30 carbon atoms.

According to an exemplary embodiment of the present specification, when adjacent R201 to R208 are bonded to each other to form a substituted or unsubstituted aromatic hydrocarbon ring group, any one of R101, R103 to R106, and R108 in Chemical Formula 1-C is bonded to L1, or any one of R101 to R108, which are not bonded to L1 in Chemical Formula 1-C, and R201 to R208, which are not bonded to L2 in Chemical Formula 1-D, is necessarily one or more of a phenyl group unsubstituted or substituted with one or more deuteriums, a naphthyl group unsubstituted or substituted with deuterium, a biphenyl group unsubstituted or substituted with deuterium, a methyl group unsubstituted or substituted with deuterium, a propyl group unsubstituted or substituted with deuterium, a tert-butyl group unsubstituted or substituted with deuterium, deuterium, or a tetrahydronaphthalene group unsubstituted or substituted with an alkyl group, or the substituted or unsubstituted ring group, which is not bonded to L2 in Chemical Formula 1-D, is substituted with a phenyl group unsubstituted or substituted with one or more deuteriums, a naphthyl group unsubstituted or substituted with deuterium, a biphenyl group unsubstituted or substituted with deuterium, a methyl group unsubstituted or substituted with deuterium, a propyl group unsubstituted or substituted with deuterium, a tert-butyl group unsubstituted or substituted with deuterium, deuterium, or a tetrahydronaphthalene group unsubstituted or substituted with an alkyl group.

According to an exemplary embodiment of the present specification, R9 of Chemical Formula 1 above is bonded to L1 of Chemical Formula 1-A above, R10 of Chemical Formula 1 above is bonded to L2 of Chemical Formula 1-B above, and Ri to R8 of Chemical Formula 1 above are each independently hydrogen; deuterium; or a phenyl group substituted or unsubstituted with deuterium.

According to an exemplary embodiment of the present specification, R9 of Chemical Formula 1 above is bonded to L1 of Chemical Formula 1-A above, R10 of Chemical Formula 1 above is bonded to L2 of Chemical Formula 1-B above, and Ri to R8 of Chemical Formula 1 above are all deuterium.

According to an exemplary embodiment of the present specification, R9 of Chemical Formula 1 above is bonded to L1 of Chemical Formula 1-A above, R10 of Chemical Formula 1 above is bonded to L2 of Chemical Formula 1-B above, and Ri to R8 of Chemical Formula 1 above are all hydrogen.

In an exemplary embodiment of the present specification, Chemical Formula 1 is an organic compound that is any one of the following compounds.

According to an exemplary embodiment of the present specification, the deuterium substitution rate of Chemical Formula 1 is 15% or more.

According to an exemplary embodiment of the present specification, the deuterium substitution rate of Chemical Formula 1 is 30% or more.

According to an exemplary embodiment of the present specification, the deuterium substitution rate of Chemical Formula 1 is 40% or more.

According to an exemplary embodiment of the present specification, the deuterium substitution rate of Chemical Formula 1 is 50% or more.

According to an exemplary embodiment of the present specification, the deuterium substitution rate of Chemical Formula 1 is 60% or more.

According to an exemplary embodiment of the present specification, the deuterium substitution rate of Chemical Formula 1 is 70% or more.

According to an exemplary embodiment of the present specification, the deuterium substitution rate of Chemical Formula 1 is 80% or more.

According to an exemplary embodiment of the present specification, the deuterium substitution rate of Chemical Formula 1 is 90% or more.

According to an exemplary embodiment of the present specification, the deuterium substitution rate of Chemical Formula 1 is 100%.

According to an exemplary embodiment of the present specification, when Chemical Formula 1 includes deuterium, there are the following effects. Specifically, the physicochemical properties related to deuterium, such as the chemical bond length, are different from those of hydrogen, the van der Waals radius of deuterium is smaller than that of hydrogen because the stretching amplitude of the C-D bond is smaller than that of the C—H bond, and in general, it can be shown that the C-D bond is shorter and stronger than the C—H bond. Therefore, when hydrogen at a substitutable position in Chemical Formula 1 is substituted with deuterium, the energy of the ground state is reduced and the bond length between deuterium and carbon is shortened, resulting in a reduction in molecular hardcore volume, and accordingly, the electrical polarizability may be reduced, and the thin film volume may be increased by weakening the intermolecular interaction. In addition, these characteristics may have an effect of reducing the crystallinity of the thin film, that is, create an amorphous state, and may be generally effective for increasing the service life and driving characteristics of an organic light emitting device, and the heat resistance may be improved compared to organic light emitting devices in the related art.

In the present specification, “including deuterium, “deuteration” or “deuterated” means that hydrogen at a substitutable position of a compound is substituted with deuterium.

In the present specification, “overdeuterated” means a compound or group in which all hydrogens in the molecule are substituted with deuterium, and has the same meaning as “100% deuterated”.

In the present specification, “X % deuterated”, “X % degree of deuteration”, or “X % deuterium substitution rate” means that X % of the hydrogens at substitutable positions in the corresponding structure are substituted with deuterium. For example, when the corresponding structure is dibenzofuran, the dibenzofuran being “25% deuterated”, “25% degree of deuteration” of the dibenzofuran, or “25% deuterium substitution rate” of the dibenzofuran means that 2 of the 8 hydrogens at the substitutable positions of the dibenzofuran are substituted with deuterium.

In the present specification, the “degree of deuteration” or “deuterium substitution rate” may be confirmed by a known method such as nuclear magnetic resonance spectroscopy (¹H NMR), thin-layer chromatography/mass spectrometry (TLS/MS), or gas chromatography/mass spectrometry (GC/MS).

Specifically, when the “degree of deuteration” or “deuterium substitution rate” is analyzed using nuclear magnetic resonance spectroscopy (¹H NMR), the degree of deuteration or deuterium substitution rate may be calculated from the integral amount of the total peak through the integration ratio on 1H NMR by adding dimethylformamide (DMF) as an internal standard.

In addition, when the “degree of deuteration” or “deuterium substitution rate” is analyzed by thin-layer chromatography/mass spectrometry (TLS/MS), the substitution rate may be calculated based on the maximum value (median value) of the distribution of molecular weights at the end of the reaction. For example, when the degree of deuteration of the following Compound A is analyzed, the molecular weight of the following starting material is set to 506 and the maximum molecular weight (median value) of the following Compound A is set to 527 in the MS graph of FIG. 3, then it may be calculated that about 81% of the hydrogen has been deuterated because 21 hydrogens of the hydrogens (26 ea) at the substitutable positions in the following starting material have been substituted with deuterium.

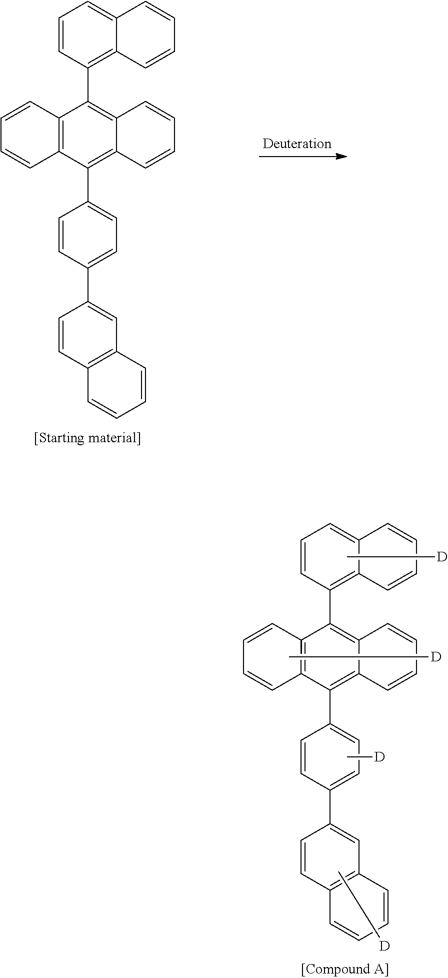

In the present specification, D means deuterium.

In an exemplary embodiment of the present specification, Chemical Formula 1 is an organic compound that is any one of the following compounds.

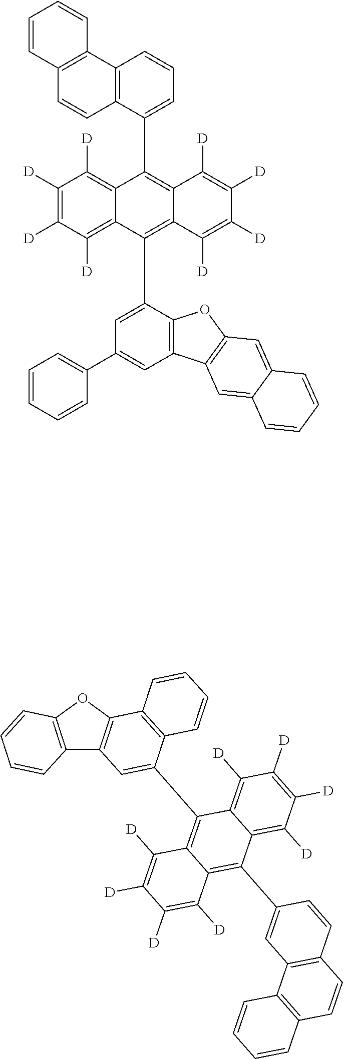 [SMILES: [2H]c1c([2H])c([2H])c2c(-c3cc(-c4ccccc4)cc4c3oc3cc5ccccc5cc34)c3c([2H])c([2H])c([2H])c([2H])c3c(-c3cccc4c3ccc3ccccc34)c2c1[2H] ; [2H]c1c([2H])c([2H])c2c(-c3cc4c5ccccc5oc4c4ccccc34)c3c([2H])c([2H])c([2H])c([2H])c3c(-c3ccc4ccc5ccccc5c4c3)c2c1[2H]]

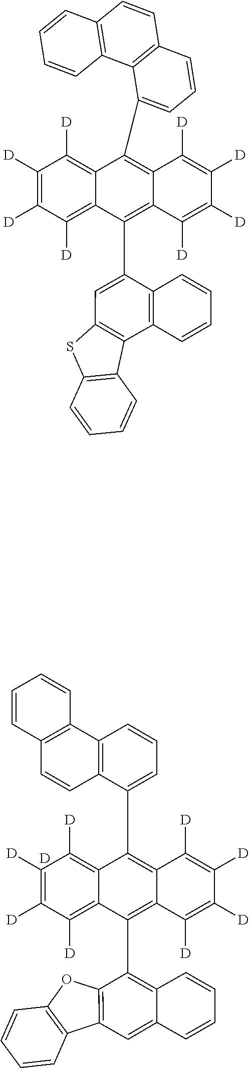

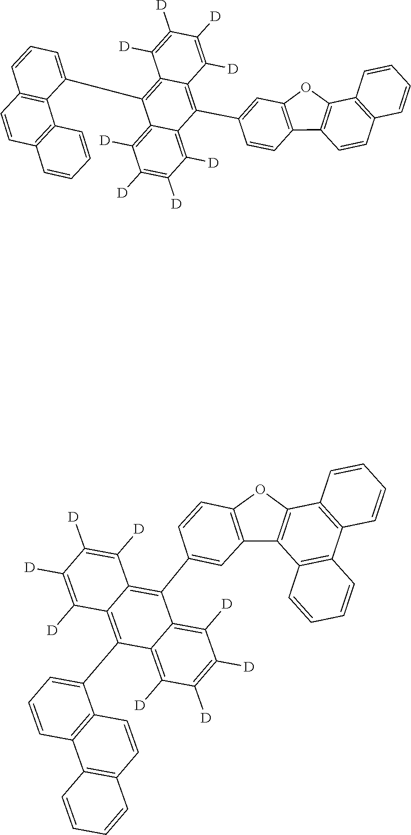 [SMILES: [2H]c1c([2H])c([2H])c2c(-c3cccc4ccc5ccccc5c34)c3c([2H])c([2H])c([2H])c([2H])c3c(-c3ccc4c(c3)oc3c5ccccc5ccc43)c2c1[2H] ; [2H]c1c([2H])c([2H])c2c(-c3cccc4c3ccc3ccccc34)c3c([2H])c([2H])c([2H])c([2H])c3c(-c3ccc4oc5c6ccccc6c6ccccc6c5c4c3)c2c1[2H]]

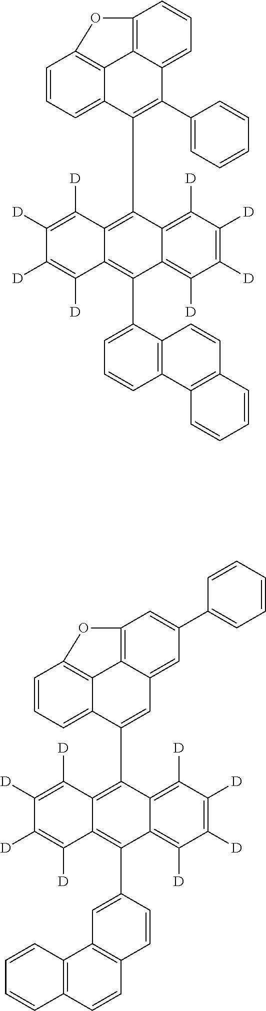 [SMILES: [2H]c1c([2H])c([2H])c2c(-c3c(-c4ccccc4)c4cccc5oc6cccc3c6c54)c3c([2H])c([2H])c([2H])c([2H])c3c(-c3cccc4c3ccc3ccccc34)c2c1[2H] ; [2H]c1c([2H])c([2H])c2c(-c3cc4cc(-c5ccccc5)cc5oc6cccc3c6c45)c3c([2H])c([2H])c([2H])c([2H])c3c(-c3ccc4ccc5ccccc5c4c3)c2c1[2H]]

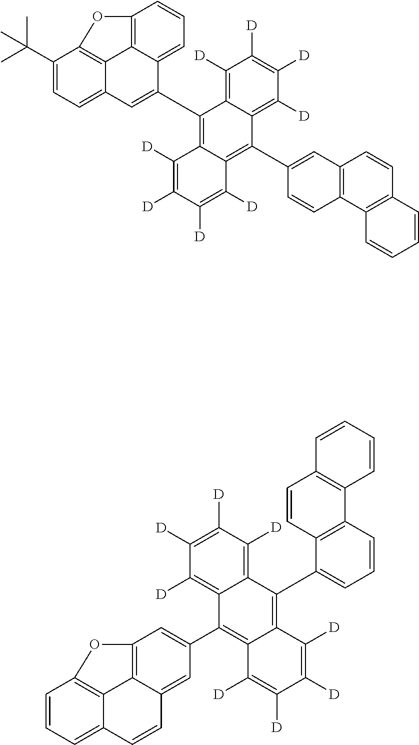 [SMILES: [2H]c1c([2H])c([2H])c2c(-c3cc4ccc(C(C)(C)C)c5oc6cccc3c6c45)c3c([2H])c([2H])c([2H])c([2H])c3c(-c3ccc4c(ccc5ccccc54)c3)c2c1[2H] ; [2H]c1c([2H])c([2H])c2c(-c3cccc4c3ccc3ccccc34)c3c([2H])c([2H])c([2H])c([2H])c3c(-c3cc4ccc5cccc6oc(c3)c4c56)c2c1[2H]]

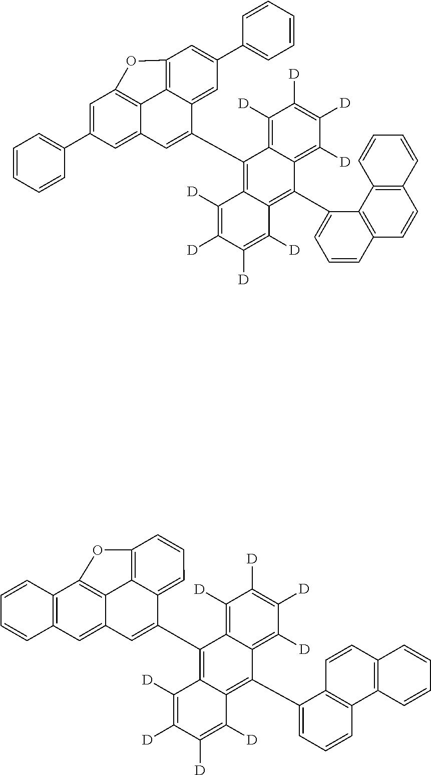 [SMILES: [2H]c1c([2H])c([2H])c2c(-c3cccc4ccc5ccccc5c34)c3c([2H])c([2H])c([2H])c([2H])c3c(-c3cc4cc(-c5ccccc5)cc5oc6cc(-c7ccccc7)cc3c6c45)c2c1[2H] ; [2H]c1c([2H])c([2H])c2c(-c3cc4cc5ccccc5c5oc6cccc3c6c45)c3c([2H])c([2H])c([2H])c([2H])c3c(-c3cccc4c3ccc3ccccc34)c2c1[2H]]

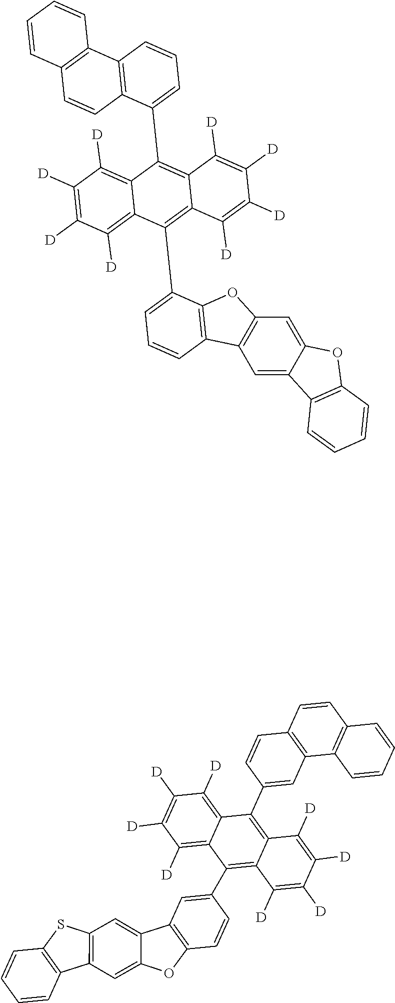 [SMILES: [2H]c1c([2H])c([2H])c2c(-c3cccc4c3oc3cc5oc6ccccc6c5cc34)c3c([2H])c([2H])c([2H])c([2H])c3c(-c3cccc4c3ccc3ccccc34)c2c1[2H] ; [2H]c1c([2H])c([2H])c2c(-c3ccc4oc5cc6c(cc5c4c3)sc3ccccc36)c3c([2H])c([2H])c([2H])c([2H])c3c(-c3ccc4ccc5ccccc5c4c3)c2c1[2H]]

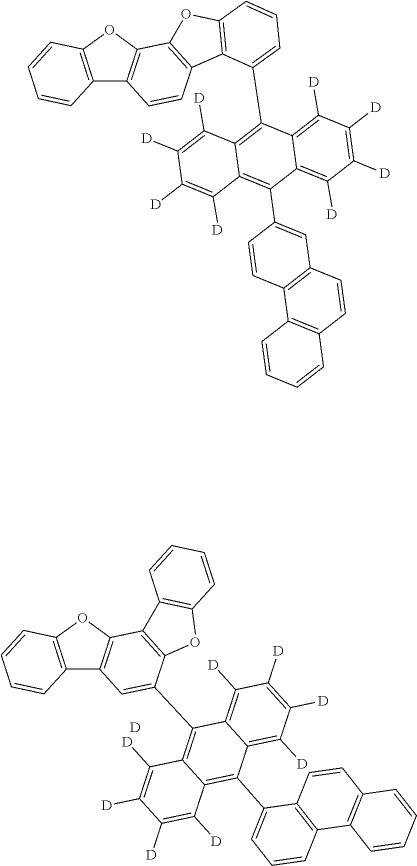

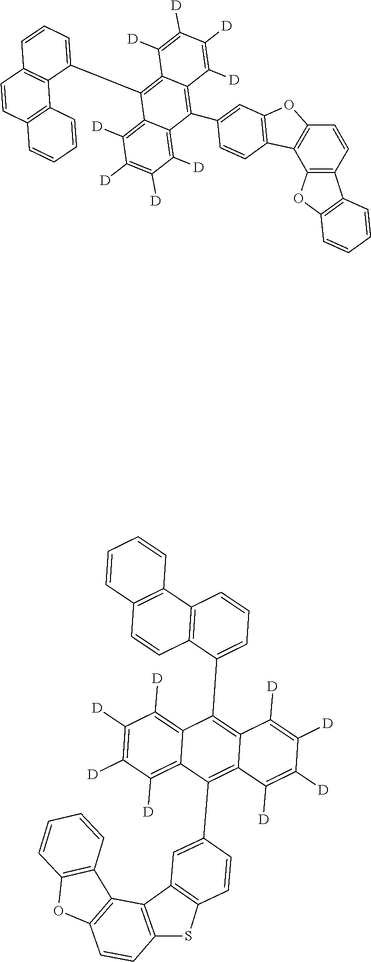 [SMILES: [2H]c1c([2H])c([2H])c2c(-c3cccc4ccc5ccccc5c34)c3c([2H])c([2H])c([2H])c([2H])c3c(-c3ccc4c(c3)oc3ccc5c6ccccc6oc5c34)c2c1[2H] ; [2H]c1c([2H])c([2H])c2c(-c3cccc4c3ccc3ccccc34)c3c([2H])c([2H])c([2H])c([2H])c3c(-c3ccc4sc5ccc6oc7ccccc7c6c5c4c3)c2c1[2H]]

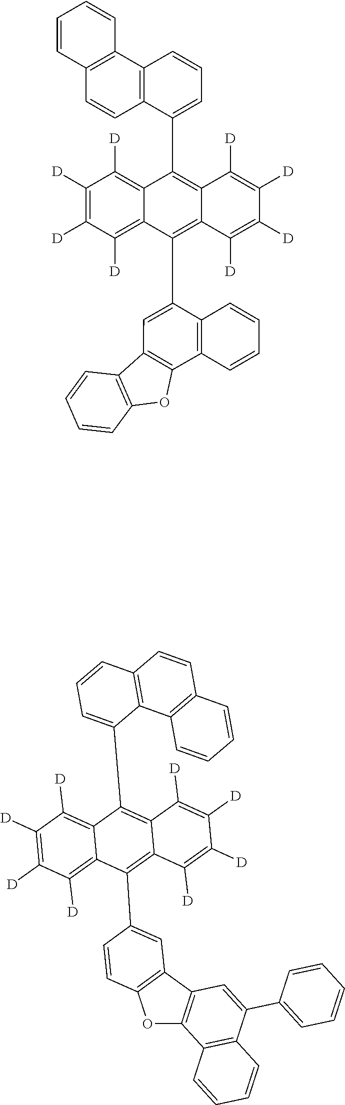 [SMILES: [2H]c1c([2H])c([2H])c2c(-c3cc4c5ccccc5oc4c4ccccc34)c3c([2H])c([2H])c([2H])c([2H])c3c(-c3cccc4c3ccc3ccccc34)c2c1[2H] ; [2H]c1c([2H])c([2H])c2c(-c3cccc4ccc5ccccc5c34)c3c([2H])c([2H])c([2H])c([2H])c3c(-c3ccc4oc5c6ccccc6c(-c6ccccc6)cc5c4c3)c2c1[2H]]

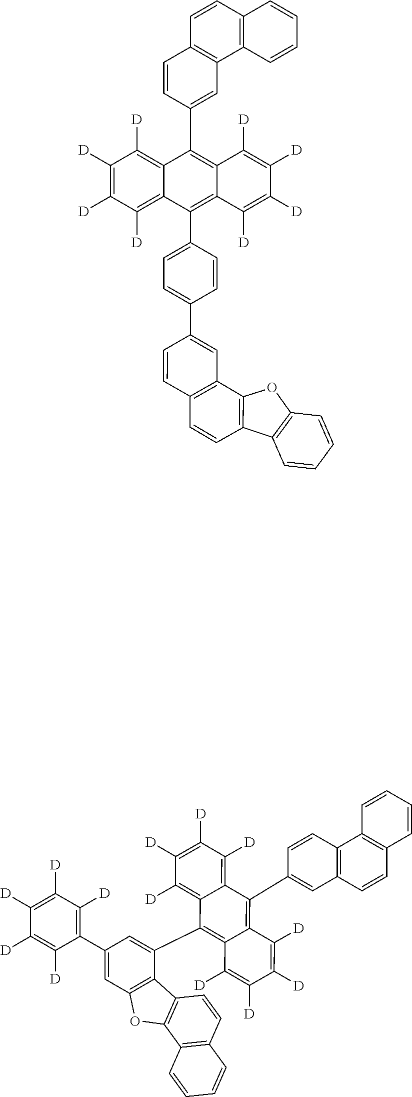 [SMILES: [2H]c1c([2H])c([2H])c2c(-c3ccc4ccc5ccccc5c4c3)c3c([2H])c([2H])c([2H])c([2H])c3c(-c3ccc(-c4ccc5ccc6c7ccccc7oc6c5c4)cc3)c2c1[2H] ; [2H]c1c([2H])c([2H])c(-c2cc(-c3c4c([2H])c([2H])c([2H])c([2H])c4c(-c4ccc5c(ccc6ccccc65)c4)c4c([2H])c([2H])c([2H])c([2H])c34)c3c(c2)oc2c4ccccc4ccc23)c([2H])c1[2H]]

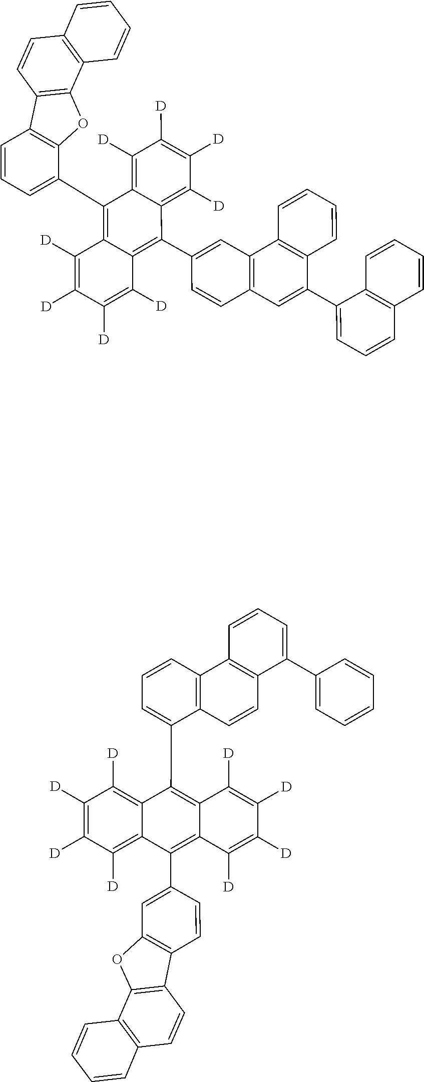 [SMILES: [2H]c1c([2H])c([2H])c2c(-c3cccc4c3oc3c5ccccc5ccc43)c3c([2H])c([2H])c([2H])c([2H])c3c(-c3ccc4cc(-c5cccc6ccccc56)c5ccccc5c4c3)c2c1[2H] ; [2H]c1c([2H])c([2H])c2c(-c3cccc4c3ccc3c(-c5ccccc5)cccc34)c3c([2H])c([2H])c([2H])c([2H])c3c(-c3ccc4c(c3)oc3c5ccccc5ccc43)c2c1[2H]]

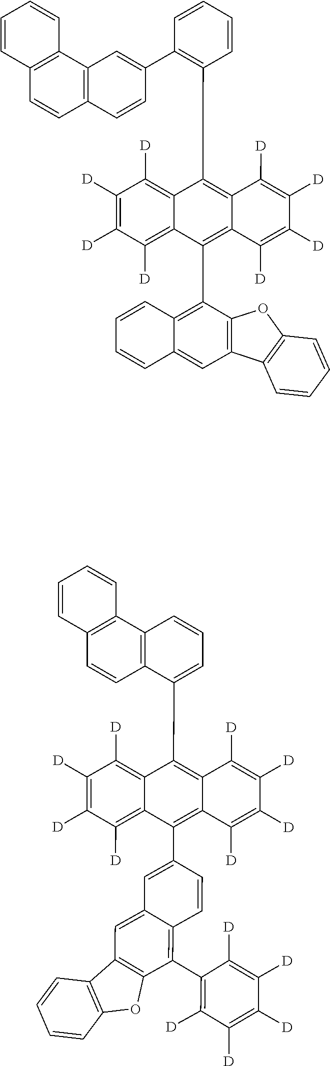 [SMILES: [2H]c1c([2H])c([2H])c2c(-c3c4ccccc4cc4c3oc3ccccc34)c3c([2H])c([2H])c([2H])c([2H])c3c(-c3ccccc3-c3ccc4ccc5ccccc5c4c3)c2c1[2H] ; [2H]c1c([2H])c([2H])c(-c2c3ccc(-c4c5c([2H])c([2H])c([2H])c([2H])c5c(-c5cccc6c5ccc5ccccc56)c5c([2H])c([2H])c([2H])c([2H])c45)cc3cc3c2oc2ccccc23)c([2H])c1[2H]]

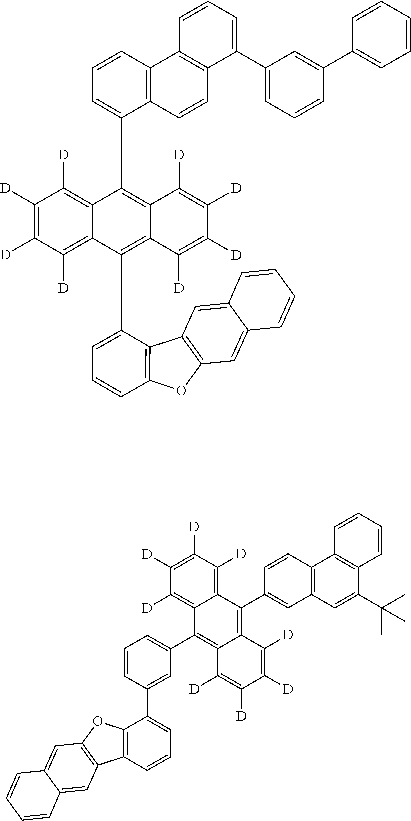 [SMILES: [2H]c1c([2H])c([2H])c2c(-c3cccc4oc5cc6ccccc6cc5c34)c3c([2H])c([2H])c([2H])c([2H])c3c(-c3cccc4c3ccc3c(-c5cccc(-c6ccccc6)c5)cccc34)c2c1[2H] ; [2H]c1c([2H])c([2H])c2c(-c3ccc4c(c3)cc(C(C)(C)C)c3ccccc34)c3c([2H])c([2H])c([2H])c([2H])c3c(-c3cccc(-c4cccc5c4oc4cc6ccccc6cc45)c3)c2c1[2H]]

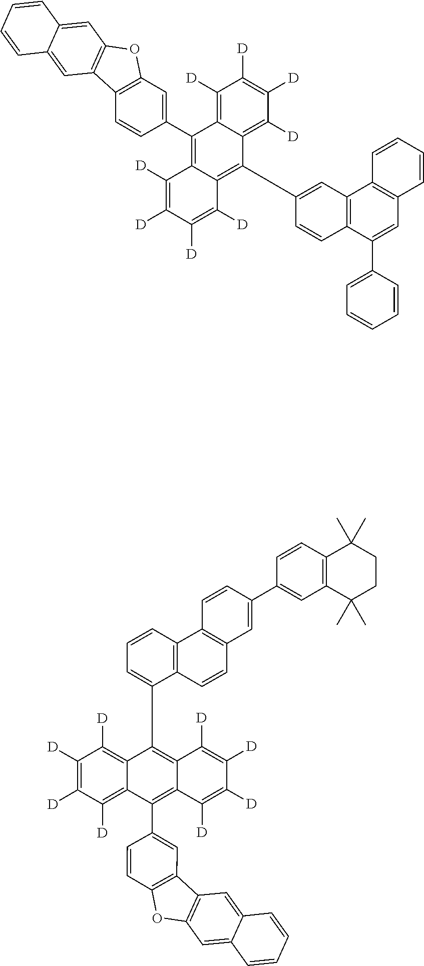 [SMILES: [2H]c1c([2H])c([2H])c2c(-c3ccc4c(-c5ccccc5)cc5ccccc5c4c3)c3c([2H])c([2H])c([2H])c([2H])c3c(-c3ccc4c(c3)oc3cc5ccccc5cc34)c2c1[2H] ; [2H]c1c([2H])c([2H])c2c(-c3cccc4c3ccc3cc(-c5ccc6c(c5)C(C)(C)CCC6(C)C)ccc34)c3c([2H])c([2H])c([2H])c([2H])c3c(-c3ccc4oc5cc6ccccc6cc5c4c3)c2c1[2H]]

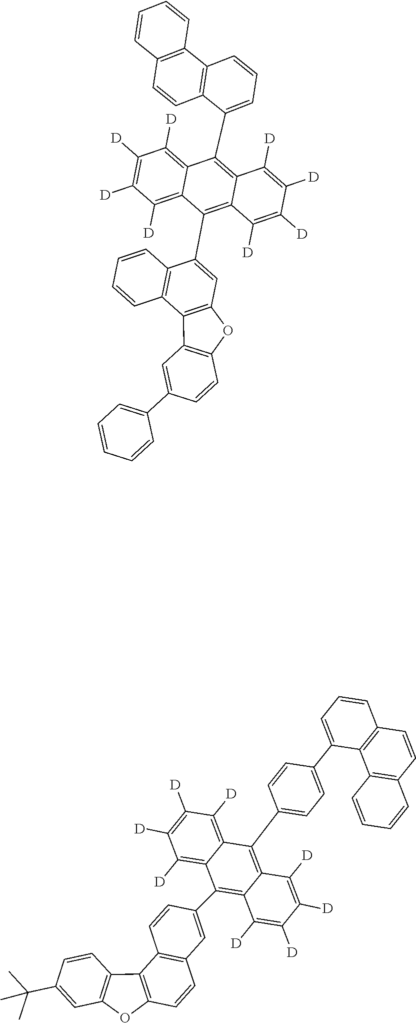 [SMILES: [2H]c1c([2H])c([2H])c2c(-c3cc4oc5ccc(-c6ccccc6)cc5c4c4ccccc34)c3c([2H])c([2H])c([2H])c([2H])c3c(-c3cccc4c3ccc3ccccc34)c2c1[2H] ; [2H]c1c([2H])c([2H])c2c(-c3ccc4c(ccc5oc6cc(C(C)(C)C)ccc6c54)c3)c3c([2H])c([2H])c([2H])c([2H])c3c(-c3ccc(-c4cccc5ccc6ccccc6c45)cc3)c2c1[2H]]

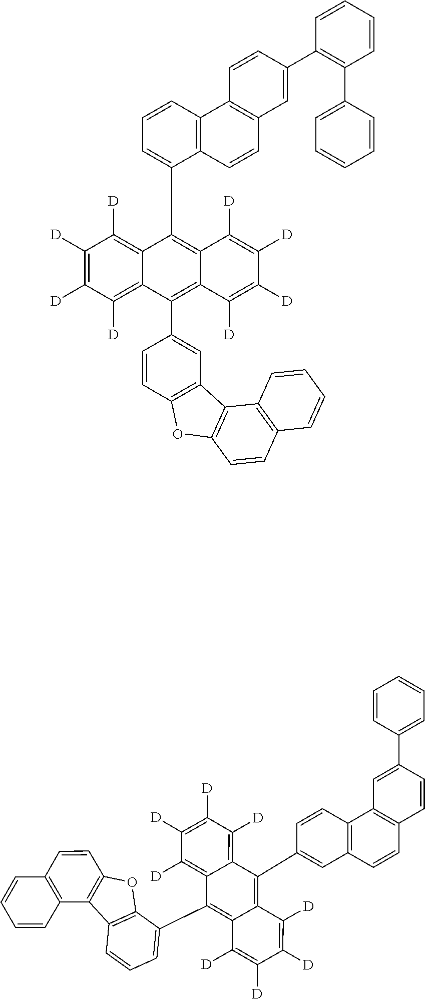 [SMILES: [2H]c1c([2H])c([2H])c2c(-c3cccc4c3ccc3cc(-c5ccccc5-c5ccccc5)ccc34)c3c([2H])c([2H])c([2H])c([2H])c3c(-c3ccc4oc5ccc6ccccc6c5c4c3)c2c1[2H] ; [2H]c1c([2H])c([2H])c2c(-c3cccc4c3oc3ccc5ccccc5c34)c3c([2H])c([2H])c([2H])c([2H])c3c(-c3ccc4c(ccc5ccc(-c6ccccc6)cc54)c3)c2c1[2H]]

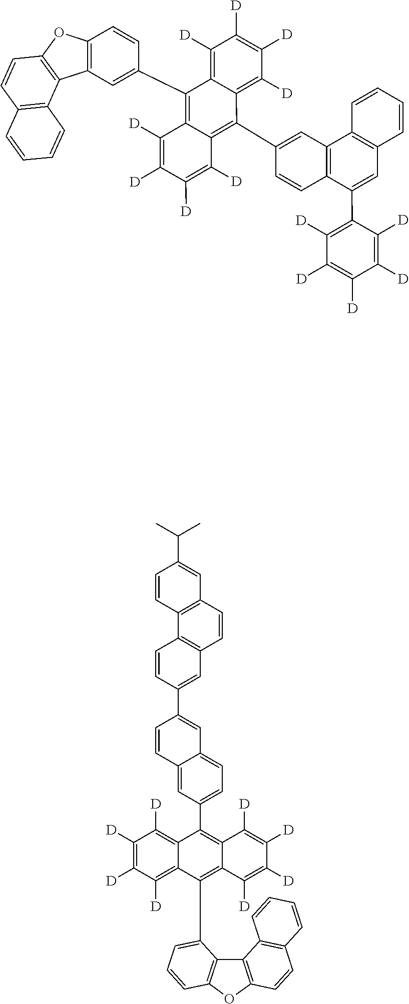 [SMILES: [2H]c1c([2H])c([2H])c(-c2cc3ccccc3c3cc(-c4c5c([2H])c([2H])c([2H])c([2H])c5c(-c5ccc6oc7ccc8ccccc8c7c6c5)c5c([2H])c([2H])c([2H])c([2H])c45)ccc23)c([2H])c1[2H] ; [2H]c1c([2H])c([2H])c2c(-c3cccc4oc5ccc6ccccc6c5c34)c3c([2H])c([2H])c([2H])c([2H])c3c(-c3ccc4cc(-c5ccc6c(ccc7cc(C(C)C)ccc76)c5)ccc4c3)c2c1[2H]]

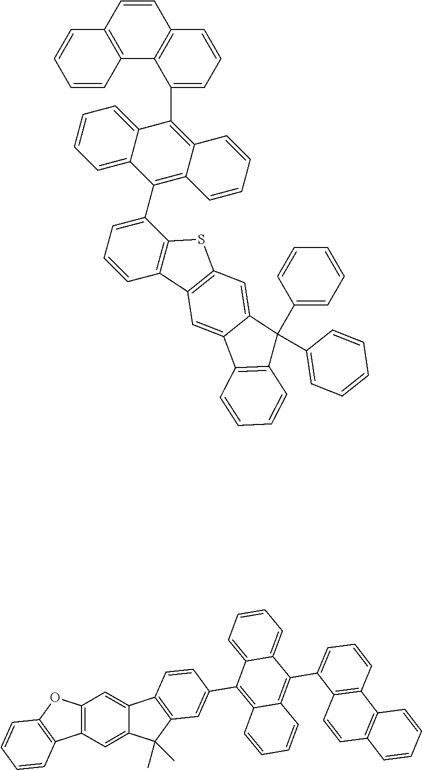 [SMILES: c1ccc(C2(c3ccccc3)c3ccccc3-c3cc4c(cc32)sc2c(-c3c5ccccc5c(-c5cccc6ccc7ccccc7c56)c5ccccc35)cccc24)cc1 ; CC1(C)c2cc(-c3c4ccccc4c(-c4cccc5c4ccc4ccccc45)c4ccccc34)ccc2-c2cc3oc4ccccc4c3cc21]

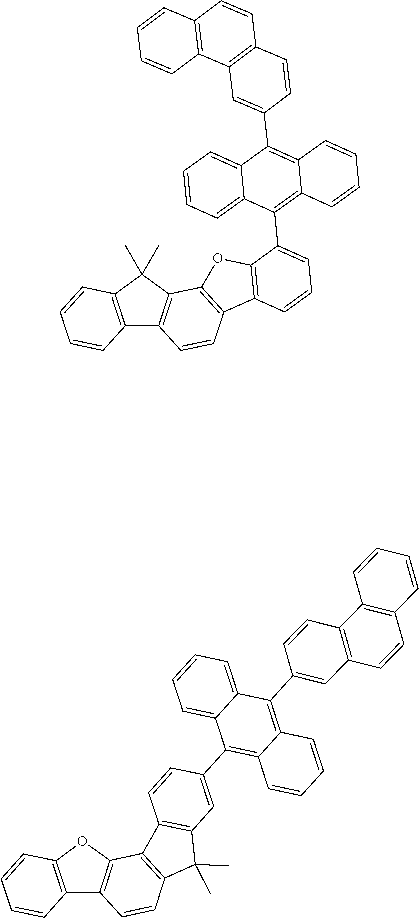 [SMILES: CC1(C)c2ccccc2-c2ccc3c(oc4c(-c5c6ccccc6c(-c6ccc7ccc8ccccc8c7c6)c6ccccc56)cccc43)c21 ; CC1(C)c2cc(-c3c4ccccc4c(-c4ccc5c(ccc6ccccc65)c4)c4ccccc34)ccc2-c2c1ccc1c2oc2ccccc21]

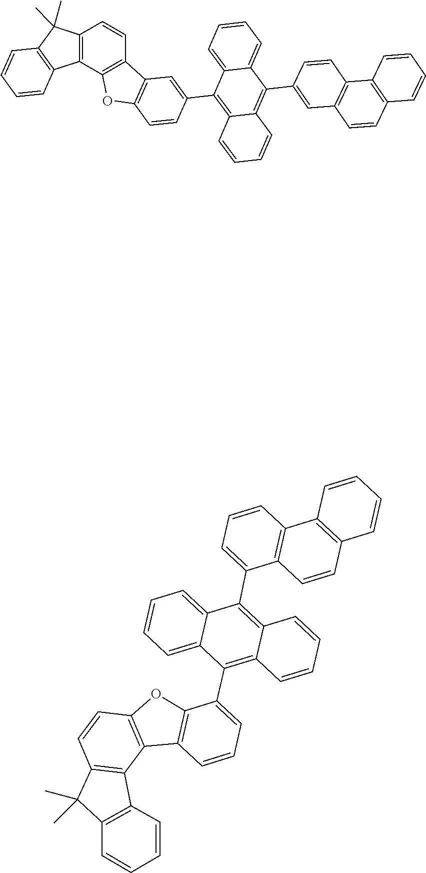 [SMILES: CC1(C)c2ccccc2-c2c1ccc1c2oc2ccc(-c3c4ccccc4c(-c4ccc5c(ccc6ccccc65)c4)c4ccccc34)cc21 ; CC1(C)c2ccccc2-c2c1ccc1oc3c(-c4c5ccccc5c(-c5cccc6c5ccc5ccccc56)c5ccccc45)cccc3c21]

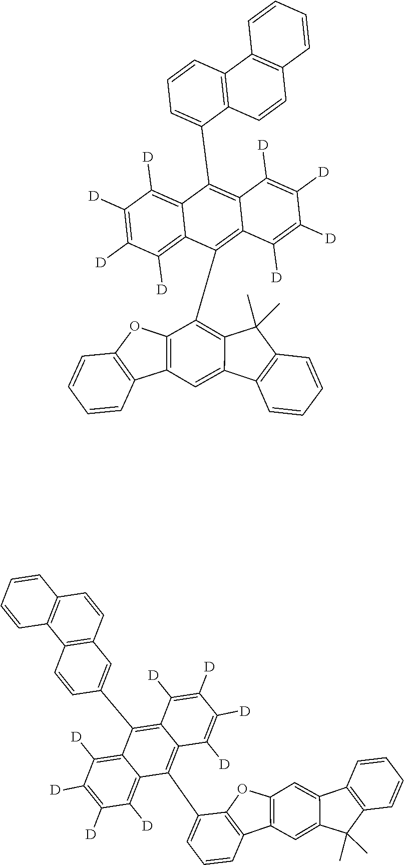 [SMILES: [2H]c1c([2H])c([2H])c2c(-c3c4c(cc5c3oc3ccccc35)-c3ccccc3C4(C)C)c3c([2H])c([2H])c([2H])c([2H])c3c(-c3cccc4c3ccc3ccccc34)c2c1[2H] ; [2H]c1c([2H])c([2H])c2c(-c3cccc4c3oc3cc5c(cc34)C(C)(C)c3ccccc3-5)c3c([2H])c([2H])c([2H])c([2H])c3c(-c3ccc4c(ccc5ccccc54)c3)c2c1[2H]]

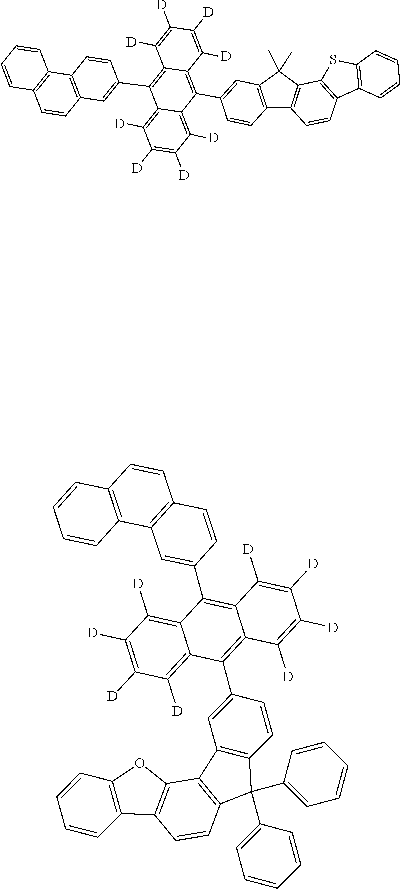 [SMILES: [2H]c1c([2H])c([2H])c2c(-c3ccc4c(ccc5ccccc54)c3)c3c([2H])c([2H])c([2H])c([2H])c3c(-c3ccc4c(c3)C(C)(C)c3c-4ccc4c3sc3ccccc34)c2c1[2H] ; [2H]c1c([2H])c([2H])c2c(-c3ccc4ccc5ccccc5c4c3)c3c([2H])c([2H])c([2H])c([2H])c3c(-c3ccc4c(c3)-c3c(ccc5c3oc3ccccc35)C4(c3ccccc3)c3ccccc3)c2c1[2H]]

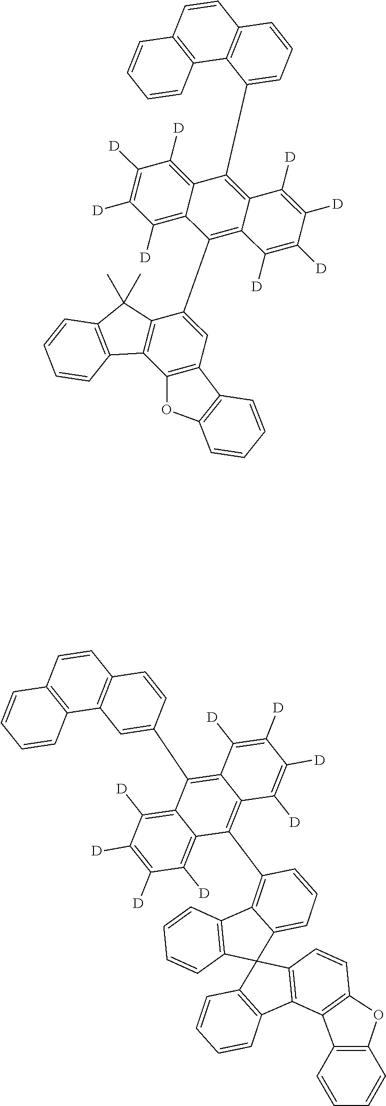 [SMILES: [2H]c1c([2H])c([2H])c2c(-c3cccc4ccc5ccccc5c34)c3c([2H])c([2H])c([2H])c([2H])c3c(-c3cc4c(oc5ccccc54)c4c3C(C)(C)c3ccccc3-4)c2c1[2H] ; [2H]c1c([2H])c([2H])c2c(-c3cccc4c3-c3ccccc3C43c4ccccc4-c4c3ccc3oc5ccccc5c43)c3c([2H])c([2H])c([2H])c([2H])c3c(-c3ccc4ccc5ccccc5c4c3)c2c1[2H]]

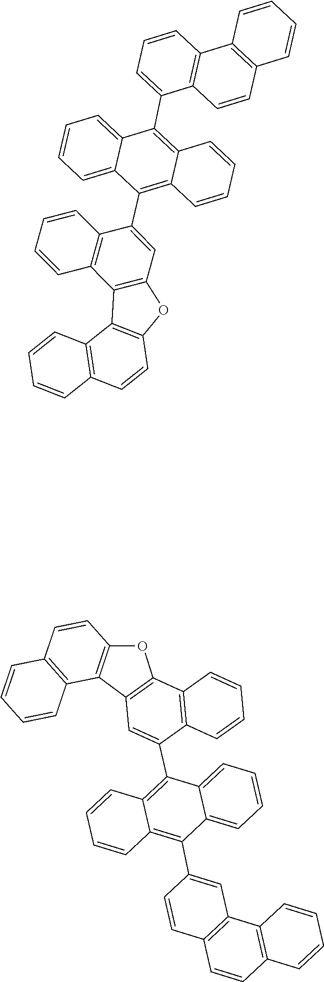 [SMILES: c1ccc2c(c1)ccc1c(-c3c4ccccc4c(-c4cc5oc6ccc7ccccc7c6c5c5ccccc45)c4ccccc34)cccc12 ; c1ccc2c(c1)ccc1ccc(-c3c4ccccc4c(-c4cc5c(oc6ccc7ccccc7c65)c5ccccc45)c4ccccc34)cc12]

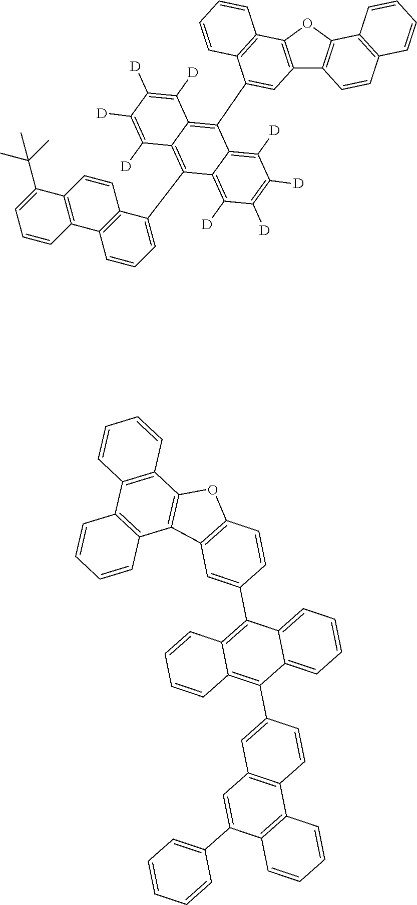 [SMILES: [2H]c1c([2H])c([2H])c2c(-c3cc4c5ccc6ccccc6c5oc4c4ccccc34)c3c([2H])c([2H])c([2H])c([2H])c3c(-c3cccc4c3ccc3c(C(C)(C)C)cccc34)c2c1[2H] ; c1ccc(-c2cc3cc(-c4c5ccccc5c(-c5ccc6oc7c8ccccc8c8ccccc8c7c6c5)c5ccccc45)ccc3c3ccccc23)cc1]

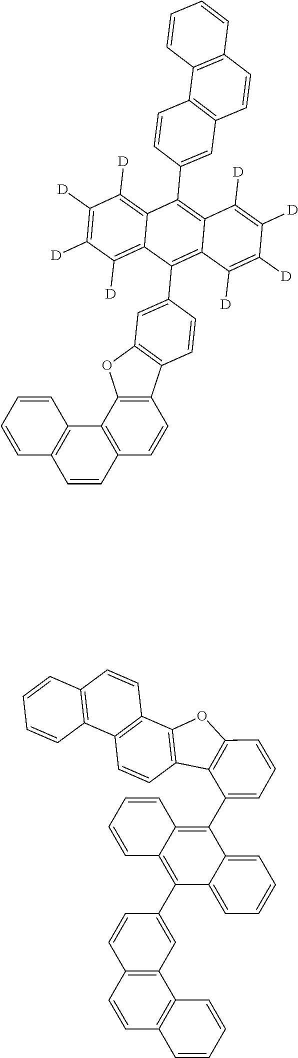 [SMILES: [2H]c1c([2H])c([2H])c2c(-c3ccc4c(c3)oc3c4ccc4ccc5ccccc5c43)c3c([2H])c([2H])c([2H])c([2H])c3c(-c3ccc4c(ccc5ccccc54)c3)c2c1[2H] ; c1ccc2c(c1)ccc1ccc(-c3c4ccccc4c(-c4cccc5oc6c7ccc8ccccc8c7ccc6c45)c4ccccc34)cc12]

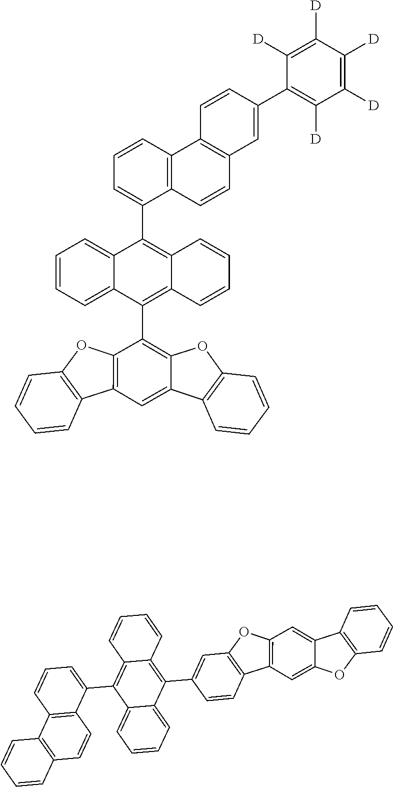 [SMILES: [2H]c1c([2H])c([2H])c(-c2ccc3c(ccc4c(-c5c6ccccc6c(-c6c7oc8ccccc8c7cc7c6oc6ccccc67)c6ccccc56)cccc43)c2)c([2H])c1[2H] ; c1ccc2c(c1)ccc1c(-c3c4ccccc4c(-c4ccc5c(c4)oc4cc6c(cc45)oc4ccccc46)c4ccccc34)cccc12]

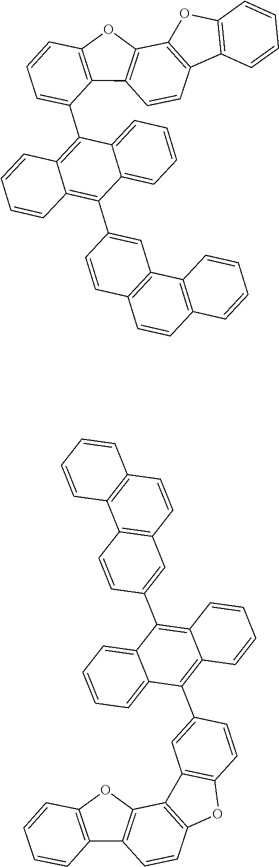 [SMILES: c1ccc2c(c1)ccc1ccc(-c3c4ccccc4c(-c4cccc5oc6c(ccc7c8ccccc8oc76)c45)c4ccccc34)cc12 ; c1ccc2c(c1)ccc1cc(-c3c4ccccc4c(-c4ccc5oc6ccc7c8ccccc8oc7c6c5c4)c4ccccc34)ccc12]

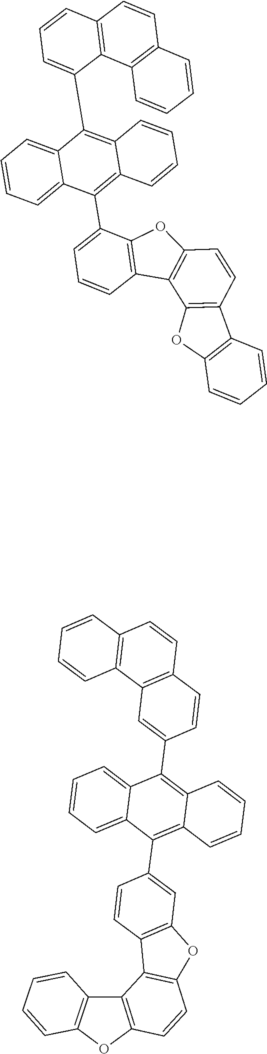 [SMILES: c1ccc2c(c1)ccc1cccc(-c3c4ccccc4c(-c4cccc5c4oc4ccc6c7ccccc7oc6c45)c4ccccc34)c12 ; c1ccc2c(c1)ccc1ccc(-c3c4ccccc4c(-c4ccc5c(c4)oc4ccc6oc7ccccc7c6c45)c4ccccc34)cc12]

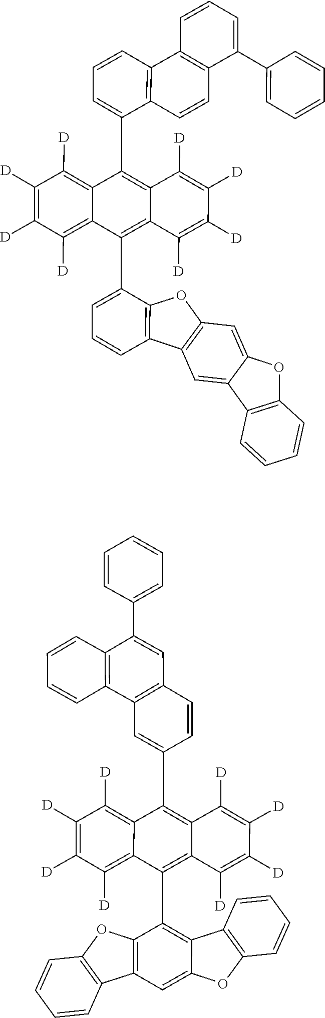 [SMILES: [2H]c1c([2H])c([2H])c2c(-c3cccc4c3oc3cc5oc6ccccc6c5cc34)c3c([2H])c([2H])c([2H])c([2H])c3c(-c3cccc4c3ccc3c(-c5ccccc5)cccc34)c2c1[2H] ; [2H]c1c([2H])c([2H])c2c(-c3c4oc5ccccc5c4cc4oc5ccccc5c34)c3c([2H])c([2H])c([2H])c([2H])c3c(-c3ccc4cc(-c5ccccc5)c5ccccc5c4c3)c2c1[2H]]

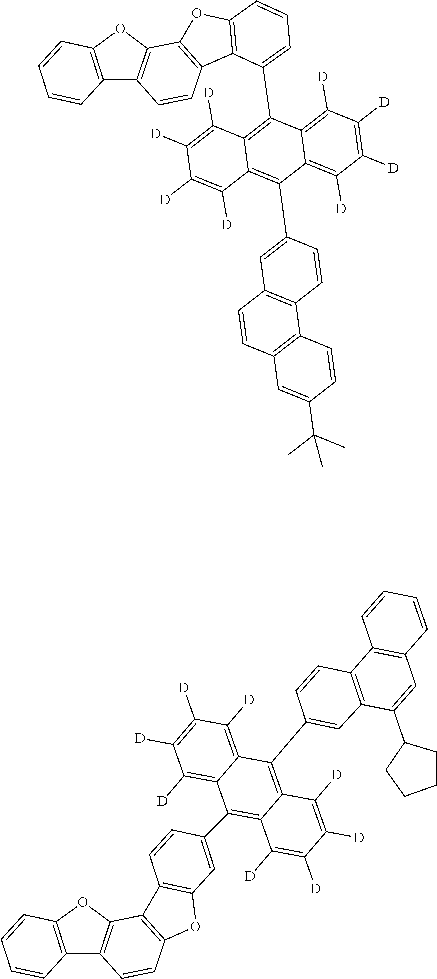 [SMILES: [2H]c1c([2H])c([2H])c2c(-c3cccc4oc5c(ccc6c7ccccc7oc65)c34)c3c([2H])c([2H])c([2H])c([2H])c3c(-c3ccc4c(ccc5cc(C(C)(C)C)ccc54)c3)c2c1[2H] ; [2H]c1c([2H])c([2H])c2c(-c3ccc4c(c3)c(C3CCCC3)cc3ccccc34)c3c([2H])c([2H])c([2H])c([2H])c3c(-c3ccc4c(c3)oc3ccc5c6ccccc6oc5c34)c2c1[2H]]

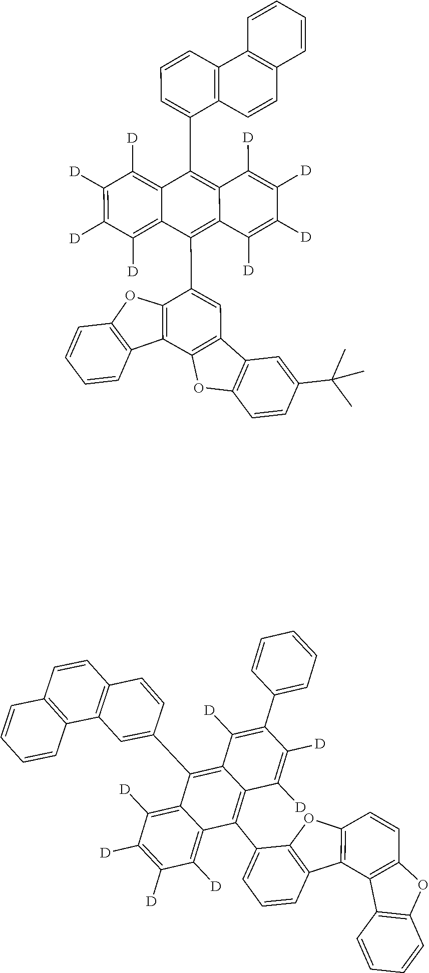 [SMILES: [2H]c1c([2H])c([2H])c2c(-c3cc4c5cc(C(C)(C)C)ccc5oc4c4c3oc3ccccc34)c3c([2H])c([2H])c([2H])c([2H])c3c(-c3cccc4c3ccc3ccccc34)c2c1[2H] ; [2H]c1c([2H])c([2H])c2c(-c3cccc4c3oc3ccc5oc6ccccc6c5c34)c3c([2H])c([2H])c(-c4ccccc4)c([2H])c3c(-c3ccc4ccc5ccccc5c4c3)c2c1[2H]]

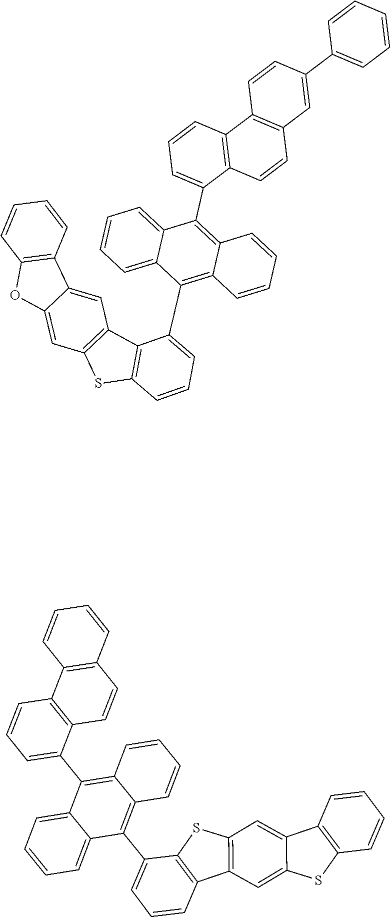 [SMILES: c1ccc(-c2ccc3c(ccc4c(-c5c6ccccc6c(-c6cccc7sc8cc9oc%10ccccc%10c9cc8c67)c6ccccc56)cccc43)c2)cc1 ; c1ccc2c(c1)ccc1c(-c3c4ccccc4c(-c4cccc5c4sc4cc6c(cc45)sc4ccccc46)c4ccccc34)cccc12]

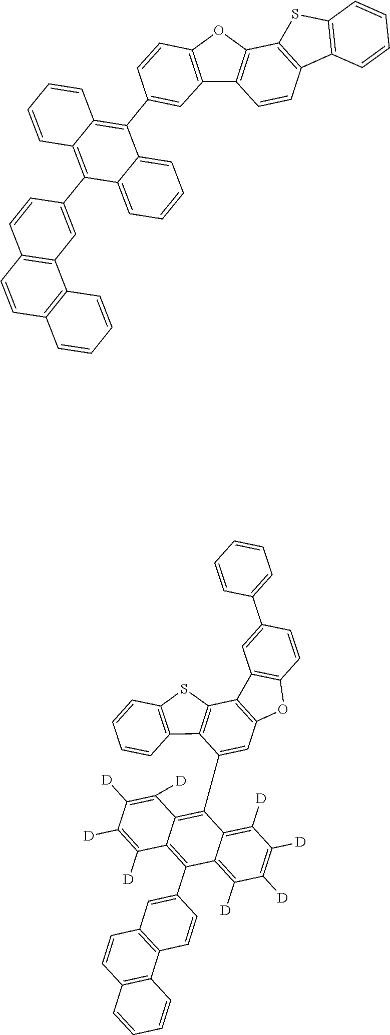 [SMILES: c1ccc2c(c1)ccc1ccc(-c3c4ccccc4c(-c4ccc5oc6c(ccc7c8ccccc8sc76)c5c4)c4ccccc34)cc12 ; [2H]c1c([2H])c([2H])c2c(-c3cc4oc5ccc(-c6ccccc6)cc5c4c4sc5ccccc5c34)c3c([2H])c([2H])c([2H])c([2H])c3c(-c3ccc4c(ccc5ccccc54)c3)c2c1[2H]]

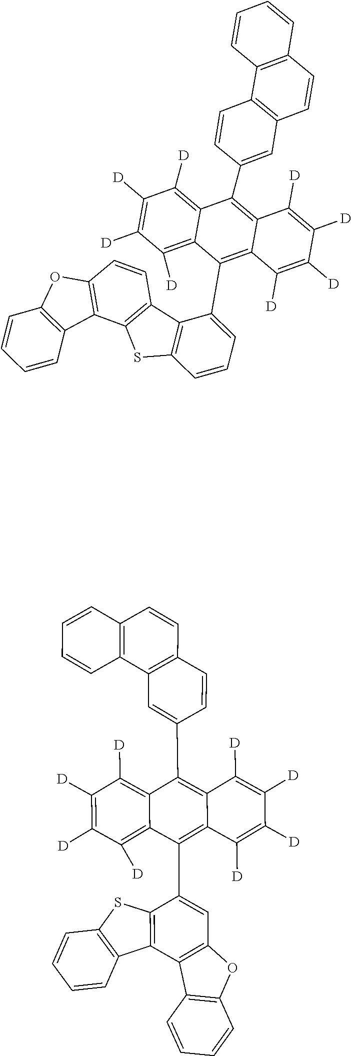 [SMILES: [2H]c1c([2H])c([2H])c2c(-c3cccc4sc5c(ccc6oc7ccccc7c65)c34)c3c([2H])c([2H])c([2H])c([2H])c3c(-c3ccc4c(ccc5ccccc54)c3)c2c1[2H] ; [2H]c1c([2H])c([2H])c2c(-c3cc4oc5ccccc5c4c4c3sc3ccccc34)c3c([2H])c([2H])c([2H])c([2H])c3c(-c3ccc4ccc5ccccc5c4c3)c2c1[2H]]

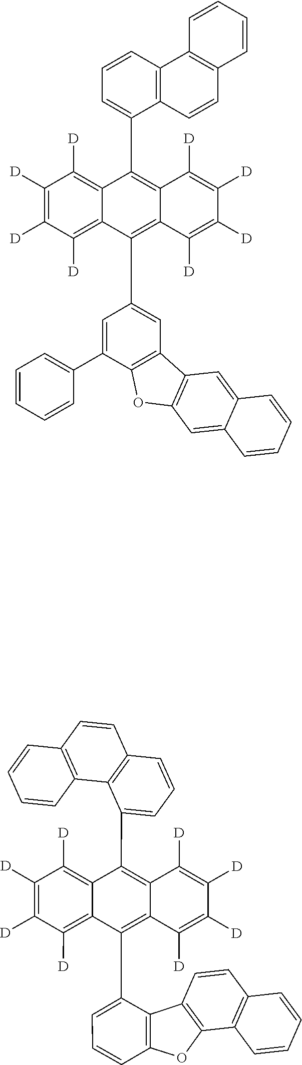 [SMILES: [2H]c1c([2H])c([2H])c2c(-c3cccc4c3ccc3ccccc34)c3c([2H])c([2H])c([2H])c([2H])c3c(-c3cc(-c4ccccc4)c4oc5cc6ccccc6cc5c4c3)c2c1[2H] ; [2H]c1c([2H])c([2H])c2c(-c3cccc4oc5c6ccccc6ccc5c34)c3c([2H])c([2H])c([2H])c([2H])c3c(-c3cccc4ccc5ccccc5c34)c2c1[2H]]

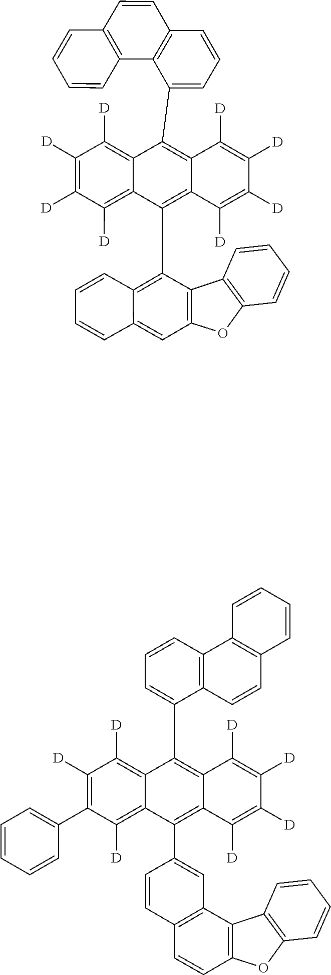 [SMILES: [2H]c1c([2H])c([2H])c2c(-c3c4ccccc4cc4oc5ccccc5c34)c3c([2H])c([2H])c([2H])c([2H])c3c(-c3cccc4ccc5ccccc5c34)c2c1[2H] ; [2H]c1c([2H])c([2H])c2c(-c3cccc4c3ccc3ccccc34)c3c([2H])c([2H])c(-c4ccccc4)c([2H])c3c(-c3ccc4ccc5oc6ccccc6c5c4c3)c2c1[2H]]

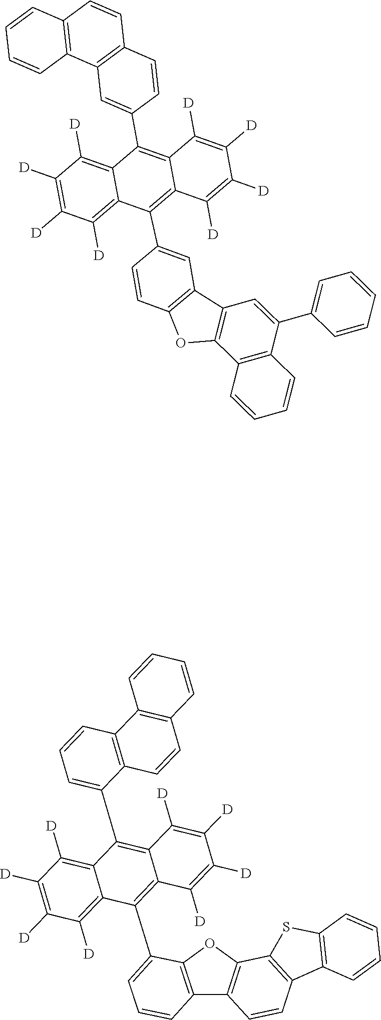 [SMILES: [2H]c1c([2H])c([2H])c2c(-c3ccc4oc5c6ccccc6c(-c6ccccc6)cc5c4c3)c3c([2H])c([2H])c([2H])c([2H])c3c(-c3ccc4ccc5ccccc5c4c3)c2c1[2H] ; [2H]c1c([2H])c([2H])c2c(-c3cccc4c3oc3c4ccc4c5ccccc5sc43)c3c([2H])c([2H])c([2H])c([2H])c3c(-c3cccc4c3ccc3ccccc34)c2c1[2H]]

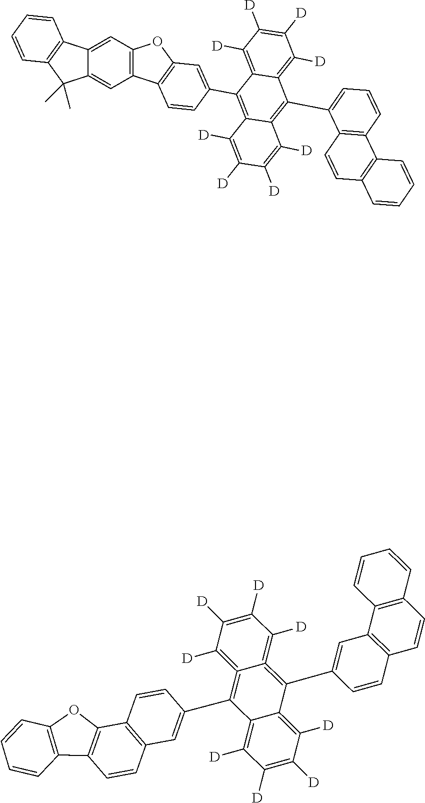

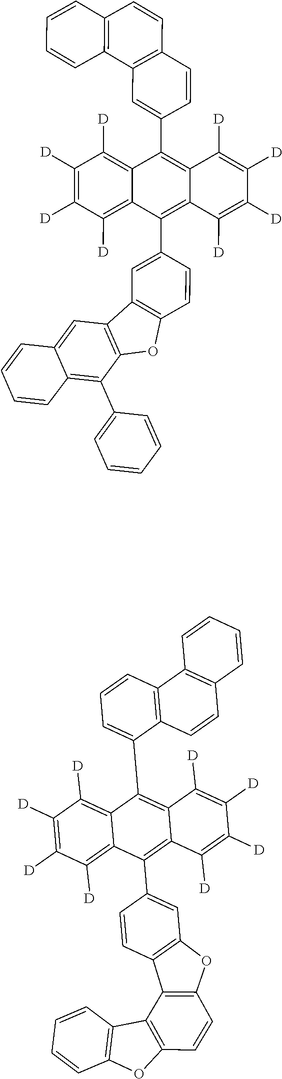 [SMILES: [2H]c1c([2H])c([2H])c2c(-c3ccc4oc5c(-c6ccccc6)c6ccccc6cc5c4c3)c3c([2H])c([2H])c([2H])c([2H])c3c(-c3ccc4ccc5ccccc5c4c3)c2c1[2H] ; [2H]c1c([2H])c([2H])c2c(-c3cccc4c3ccc3ccccc34)c3c([2H])c([2H])c([2H])c([2H])c3c(-c3ccc4c(c3)oc3ccc5oc6ccccc6c5c34)c2c1[2H]]

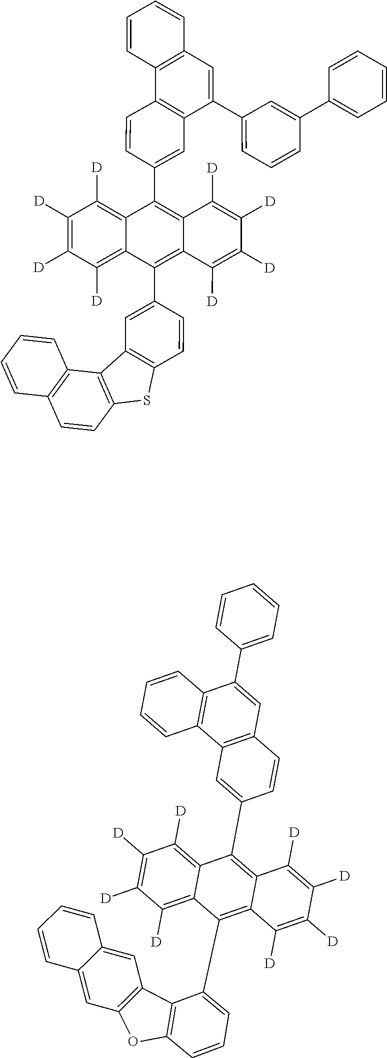 [SMILES: [2H]c1c([2H])c([2H])c2c(-c3ccc4sc5ccc6ccccc6c5c4c3)c3c([2H])c([2H])c([2H])c([2H])c3c(-c3ccc4c(c3)c(-c3cccc(-c5ccccc5)c3)cc3ccccc34)c2c1[2H] ; [2H]c1c([2H])c([2H])c2c(-c3cccc4oc5cc6ccccc6cc5c34)c3c([2H])c([2H])c([2H])c([2H])c3c(-c3ccc4cc(-c5ccccc5)c5ccccc5c4c3)c2c1[2H]]

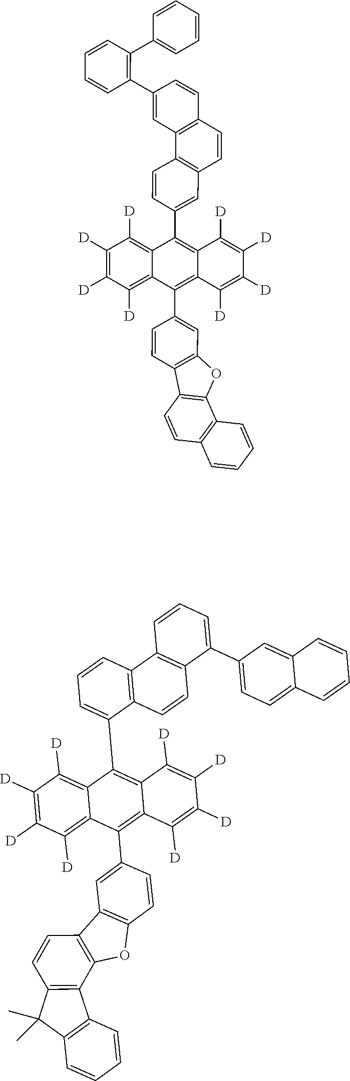 [SMILES: [2H]c1c([2H])c([2H])c2c(-c3ccc4c(c3)oc3c5ccccc5ccc43)c3c([2H])c([2H])c([2H])c([2H])c3c(-c3ccc4c(ccc5ccc(-c6ccccc6-c6ccccc6)cc54)c3)c2c1[2H] ; [2H]c1c([2H])c([2H])c2c(-c3cccc4c3ccc3c(-c5ccc6ccccc6c5)cccc34)c3c([2H])c([2H])c([2H])c([2H])c3c(-c3ccc4oc5c6c(ccc5c4c3)C(C)(C)c3ccccc3-6)c2c1[2H]]

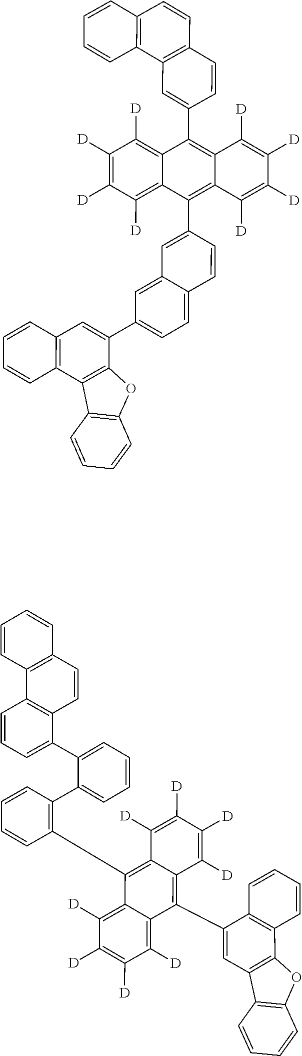 [SMILES: [2H]c1c([2H])c([2H])c2c(-c3ccc4ccc5ccccc5c4c3)c3c([2H])c([2H])c([2H])c([2H])c3c(-c3ccc4ccc(-c5cc6ccccc6c6c5oc5ccccc56)cc4c3)c2c1[2H] ; [2H]c1c([2H])c([2H])c2c(-c3cc4c5ccccc5oc4c4ccccc34)c3c([2H])c([2H])c([2H])c([2H])c3c(-c3ccccc3-c3ccccc3-c3cccc4c3ccc3ccccc34)c2c1[2H]]

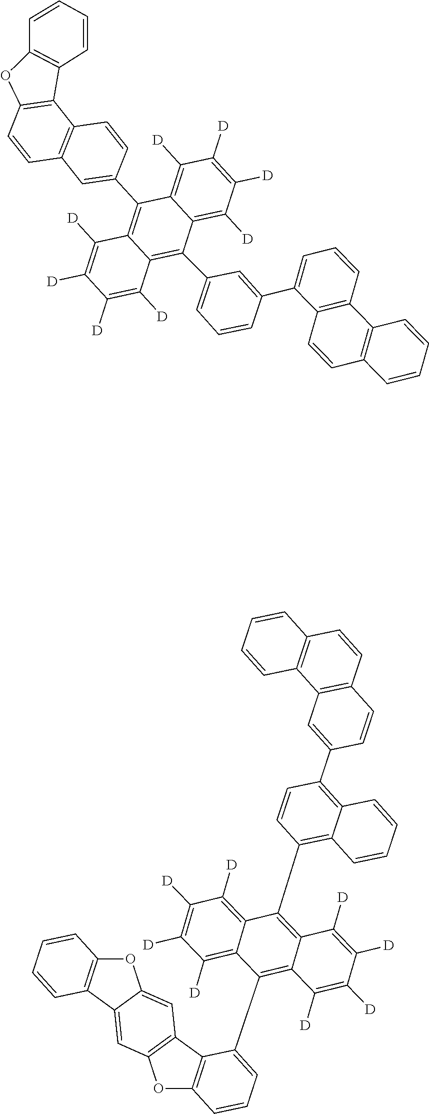 [SMILES: [2H]c1c([2H])c([2H])c2c(-c3ccc4c(ccc5oc6ccccc6c54)c3)c3c([2H])c([2H])c([2H])c([2H])c3c(-c3cccc(-c4cccc5c4ccc4ccccc45)c3)c2c1[2H] ; [2H]c1c([2H])c([2H])c2c(-c3cccc4oc5cc6c(cc5c34)oc3ccccc36)c3c([2H])c([2H])c([2H])c([2H])c3c(-c3ccc(-c4ccc5ccc6ccccc6c5c4)c4ccccc34)c2c1[2H]]

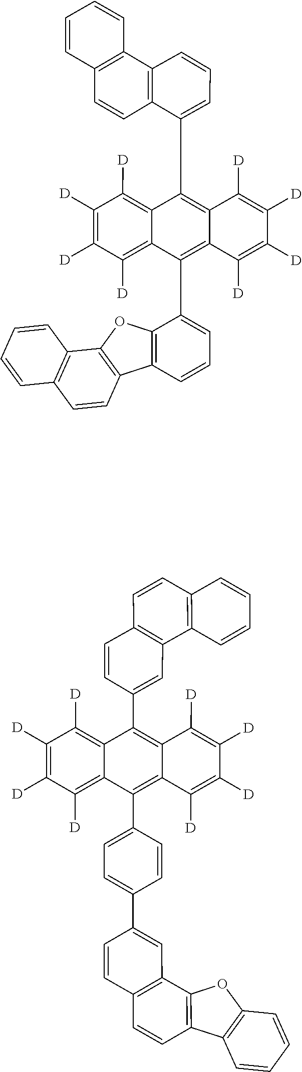 [SMILES: [2H]c1c([2H])c([2H])c2c(-c3cccc4c3oc3c5ccccc5ccc43)c3c([2H])c([2H])c([2H])c([2H])c3c(-c3cccc4c3ccc3ccccc34)c2c1[2H] ; [2H]c1c([2H])c([2H])c2c(-c3ccc4ccc5ccccc5c4c3)c3c([2H])c([2H])c([2H])c([2H])c3c(-c3ccc(-c4ccc5ccc6c7ccccc7oc6c5c4)cc3)c2c1[2H]]

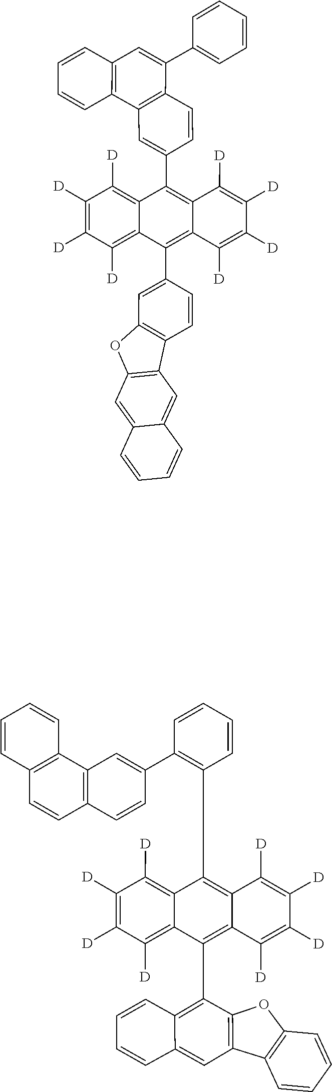 [SMILES: [2H]c1c([2H])c([2H])c2c(-c3ccc4c(-c5ccccc5)cc5ccccc5c4c3)c3c([2H])c([2H])c([2H])c([2H])c3c(-c3ccc4c(c3)oc3cc5ccccc5cc34)c2c1[2H] ; [2H]c1c([2H])c([2H])c2c(-c3c4ccccc4cc4c3oc3ccccc34)c3c([2H])c([2H])c([2H])c([2H])c3c(-c3ccccc3-c3ccc4ccc5ccccc5c4c3)c2c1[2H]]

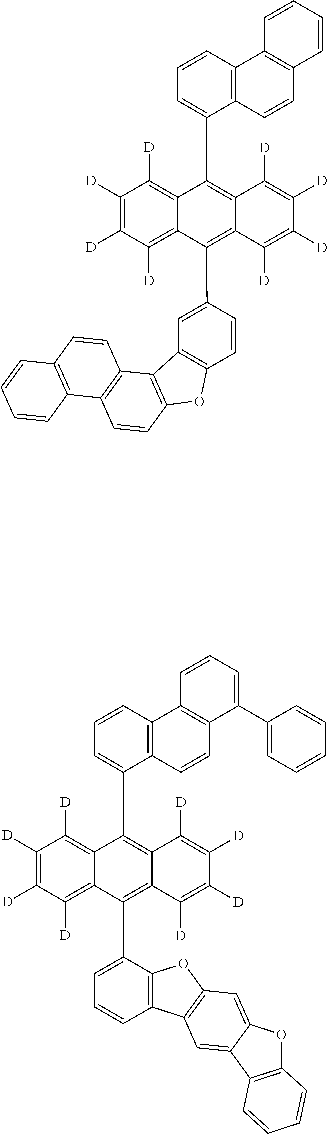 [SMILES: [2H]c1c([2H])c([2H])c2c(-c3cccc4c3ccc3ccccc34)c3c([2H])c([2H])c([2H])c([2H])c3c(-c3ccc4oc5ccc6c7ccccc7ccc6c5c4c3)c2c1[2H] ; [2H]c1c([2H])c([2H])c2c(-c3cccc4c3oc3cc5oc6ccccc6c5cc34)c3c([2H])c([2H])c([2H])c([2H])c3c(-c3cccc4c3ccc3c(-c5ccccc5)cccc34)c2c1[2H]]

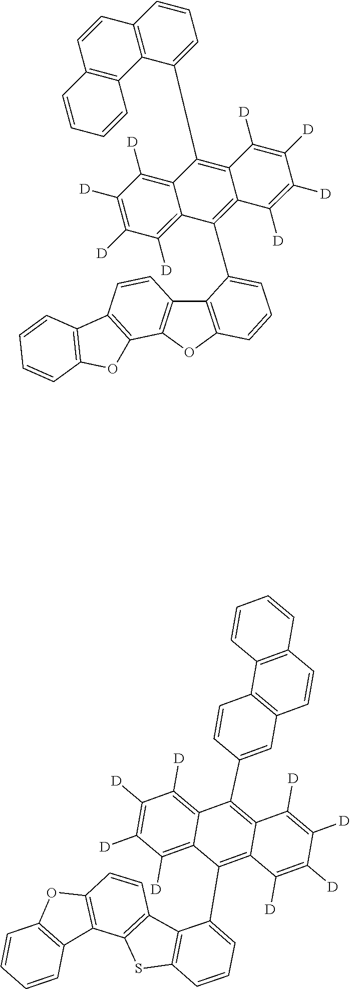 [SMILES: [2H]c1c([2H])c([2H])c2c(-c3cccc4oc5c(ccc6c7ccccc7oc65)c34)c3c([2H])c([2H])c([2H])c([2H])c3c(-c3cccc4ccc5ccccc5c34)c2c1[2H] ; [2H]c1c([2H])c([2H])c2c(-c3cccc4sc5c(ccc6oc7ccccc7c65)c34)c3c([2H])c([2H])c([2H])c([2H])c3c(-c3ccc4c(ccc5ccccc54)c3)c2c1[2H]]

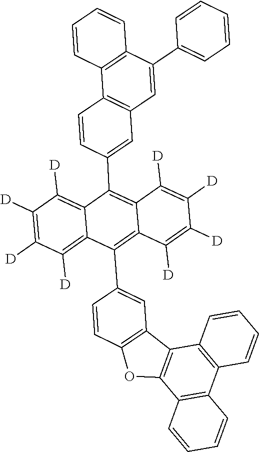 [SMILES: [2H]c1c([2H])c([2H])c2c(-c3ccc4oc5c6ccccc6c6ccccc6c5c4c3)c3c([2H])c([2H])c([2H])c([2H])c3c(-c3ccc4c(c3)cc(-c3ccccc3)c3ccccc34)c2c1[2H]]

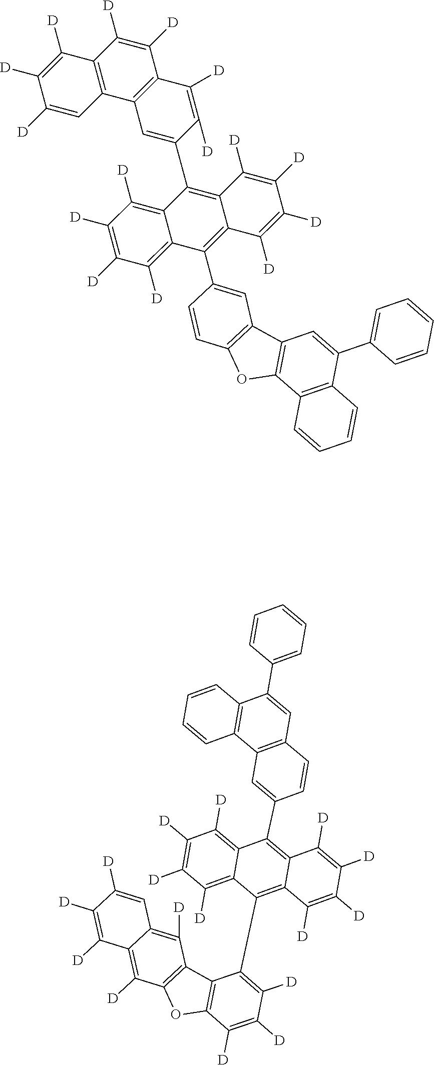 [SMILES: [2H]c1cc2c(c([2H])c1[2H])c([2H])c([2H])c1c([2H])c([2H])c(-c3c4c([2H])c([2H])c([2H])c([2H])c4c(-c4ccc5oc6c7ccccc7c(-c7ccccc7)cc6c5c4)c4c([2H])c([2H])c([2H])c([2H])c34)cc12 ; [2H]c1cc2c([2H])c3c(oc4c([2H])c([2H])c([2H])c(-c5c6c([2H])c([2H])c([2H])c([2H])c6c(-c6ccc7cc(-c8ccccc8)c8ccccc8c7c6)c6c([2H])c([2H])c([2H])c([2H])c56)c43)c([2H])c2c([2H])c1[2H]]

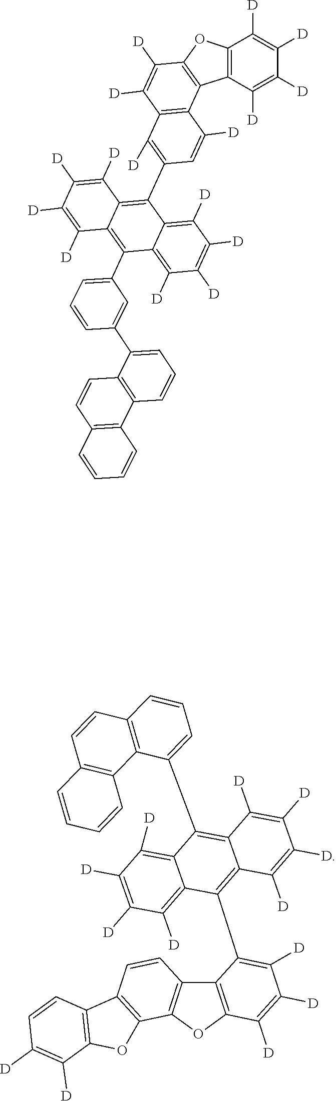

In the organic compound of Chemical Formula 1 according to an exemplary embodiment of the present specification, a core structure may be prepared as in the Preparation Examples described below. The substituent may be bonded by a method known in the art, and the kind and position of the substituent or the number of substituents may be changed according to the technology known in the art.

In the present specification, compounds having various energy bandgaps may be synthesized by introducing various substituents into the core structure of the organic compound of Chemical Formula 1. Further, in the present specification, various substituents may be introduced into the core structures having the structure described above to adjust the HOMO and LUMO energy levels of a compound.

In addition, the present specification provides an organic light emitting device including the above-described organic compound.

When one member is disposed “on” another member in the present specification, this includes not only a case where the one member is brought into contact with another member, but also a case where still another member is present between the two members.

The organic light emitting device according to the present specification is an organic light emitting device including: an anode; a cathode; and an organic material layer having one or more layers provided between the anode and the cathode, in which one or more layers of the organic material layer include the above-described organic compound represented by Chemical Formula 1.

The organic material layer of the organic light emitting device of the present specification may be composed of a single-layered structure, but may also be composed of a multi-layered structure in which two or more organic material layers are stacked. For example, the organic light emitting device of the present invention may have a structure including one or more layers of a hole transport layer, a hole injection layer, an electron adjusting layer, a hole transport and injection layer, an electron transport layer, an electron injection layer, a hole adjusting layer, and an electron transport and injection layer as organic material layers. However, the structure of the organic light emitting device of the present specification is not limited thereto, and may include a fewer or greater number of organic material layers.

In the organic light emitting device of the present specification, the organic light emitting device includes a light emitting layer, and the light emitting layer may include the organic compound.

For example, the organic compound of Chemical Formula 1 may be included as a host of the light emitting layer.

According to an exemplary embodiment of the present specification, the light emitting layer includes a dopant, and the dopant includes a fluorescent dopant.

According to an exemplary embodiment of the present specification, the fluorescent dopant is a pyrene-based compound or a bipyrene-based compound.

According to an exemplary embodiment of the present specification, the bipyrene-based compound includes a boron-based compound.

According to an exemplary embodiment of the present specification, the light emitting layer further includes one or more hosts different from the compound of Chemical Formula 1.

In an exemplary embodiment of the present specification, the host different from the compound of Chemical Formula 1 includes a compound of the following Chemical Formula H-1.

In an exemplary embodiment of the present specification, the light emitting layer of the organic light emitting device includes the organic compound as a host and further includes one host represented by the following Chemical Formula H-1 different from the organic compound.

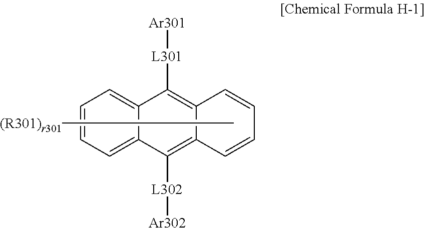

In Chemical Formula H-1,
L301 and L302 are the same as or different from each other, and are each independently a direct bond; a substituted or unsubstituted arylene group; or a substituted or unsubstituted heterocyclic group,
Ar301 and Ar302 are the same as or different from each other, and are each independently hydrogen; deuterium; a substituted or unsubstituted aryl group; or a substituted or unsubstituted heterocyclic group,
R301 is hydrogen; deuterium; a halogen group; a substituted or unsubstituted alkyl group; a substituted or unsubstituted cycloalkyl group; a substituted or unsubstituted aryl group; or a substituted or unsubstituted heterocyclic group, and
r301 is an integer from 1 to 7, and when r301 is 2 or higher, two or more R301's are the same as or different from each other.

According to an exemplary embodiment of the present specification, Chemical Formula H-1 is represented by the following compound.

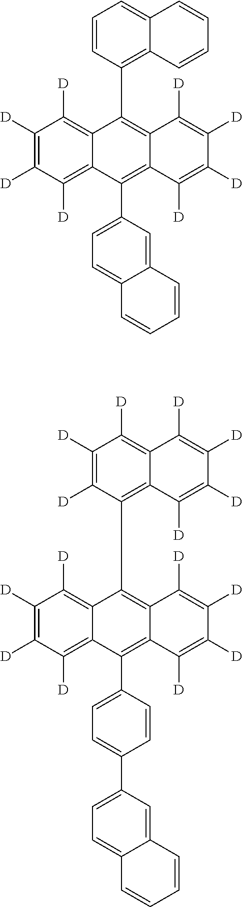 [SMILES: [2H]c1c([2H])c([2H])c2c(-c3cccc4ccccc34)c3c([2H])c([2H])c([2H])c([2H])c3c(-c3ccc4ccccc4c3)c2c1[2H] ; [2H]c1c([2H])c([2H])c2c(-c3c4c([2H])c([2H])c([2H])c([2H])c4c(-c4ccc(-c5ccc6ccccc6c5)cc4)c4c([2H])c([2H])c([2H])c([2H])c34)c([2H])c([2H])c([2H])c2c1[2H]]

Any anthracene-based host used in the art can be used without limitation as long as the host different from the compound of Chemical Formula 1 is different from Chemical Formula 1, and the host is not limited thereto.

According to an exemplary embodiment of the present specification, the light emitting layer includes a host and a dopant.

According to an exemplary embodiment of the present specification, the light emitting layer includes a host and a dopant, and the host includes the compound represented by Chemical Formula 1.

According to an exemplary embodiment of the present specification, the dopant is a blue dopant.

According to an exemplary embodiment of the present specification, the organic light emitting device is a blue organic light emitting device.

According to an exemplary embodiment of the present specification, the light emitting layer includes a host and a dopant, the host includes the compound represented by Chemical Formula 1, and the dopant includes one or more selected from a pyrene-based compound and a bipyrene-based compound.

According to an exemplary embodiment of the present specification, the light emitting layer includes a host and a dopant, and for the light emitting layer, the host and the dopant has a weight ratio of 8:2 to 99:1.

According to an exemplary embodiment of the present specification, the light emitting layer includes a host and a dopant, and for the light emitting layer, the host and the dopant has a weight ratio of 8:2 to 95:5.

According to an exemplary embodiment of the present specification, the light emitting layer includes a host and a dopant, and for the light emitting layer, the host and the dopant have a weight ratio of 90:10 to 99:1.

According to another exemplary embodiment of the present specification, the light emitting layer further includes a capping layer provided on a surface of at least one of the first electrode and the second electrode opposite to the surface facing the organic material layer.

The capping layer is formed to prevent a considerable amount of light from being lost through total reflection of light in the organic light emitting device, and the capping layer has the performance capable of sufficiently protecting the underlying negative electrode and light emitting layer from external moisture penetration or contamination, and has a high refractive index, and thus may prevent light loss caused by total reflection.

According to still another exemplary embodiment of the present specification, the capping layer may be provided on each of a surface of the first electrode opposite to the surface facing the organic layer and a surface of the second electrode opposite to the surface facing the organic layer.

According to yet another exemplary embodiment of the present specification, the capping layer may be provided on a surface of the first electrode opposite to the surface facing the organic layer.

According to still yet another exemplary embodiment of the present specification, the capping layer may be provided on a surface of the second electrode opposite to the surface facing the organic layer.

According to an exemplary embodiment of the present specification, the organic light emitting device further includes one or two or more layers selected from the group consisting of a hole injection layer, a hole transport layer, a light emitting layer, an electron transport layer, an electron injection layer, an electron adjusting layer, and a hole adjusting layer.

According to an exemplary embodiment of the present specification, the organic light emitting device includes: a first electrode; a second electrode; a light emitting layer provided between the first electrode and the second electrode; and an organic material layer having two or more layers provided between the light emitting layer and the first electrode, or between the light emitting layer and the second electrode.

According to an exemplary embodiment of the present specification, as the organic material layer having two or more layers between the light emitting layer and the first electrode, or between the light emitting layer and the second electrode, two or more may be selected from the group consisting of a light emitting layer, a hole transport layer, a hole injection layer, a hole injection and transport layer, a hole adjusting layer, an electron adjusting layer, an electron injection layer, an electron transport layer, and an electron injection and transport layer.

According to an exemplary embodiment of the present specification, a hole transport layer having two or more layers is included between the light emitting layer and the first electrode. The hole transport layer having two or more layers may include materials which are the same as or different from each other.

According to an exemplary embodiment of the present specification, the first electrode is an anode or a cathode.

According to an exemplary embodiment of the present specification, the second electrode is a cathode or an anode.

According to an exemplary embodiment of the present specification, the organic light emitting device may be a normal type organic light emitting device in which an anode, an organic material layer having one or more layers, and a cathode are sequentially stacked on a substrate.

According to an exemplary embodiment of the present specification, the organic light emitting device may be an inverted type organic light emitting device in which a cathode, an organic material layer having one or more layers, and an anode are sequentially stacked on a substrate.

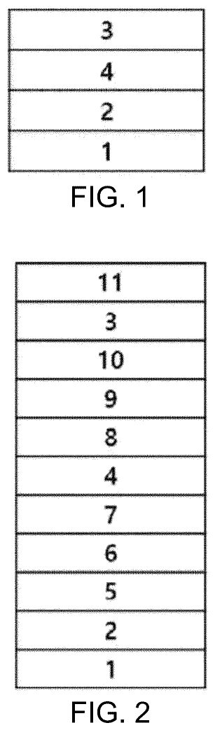

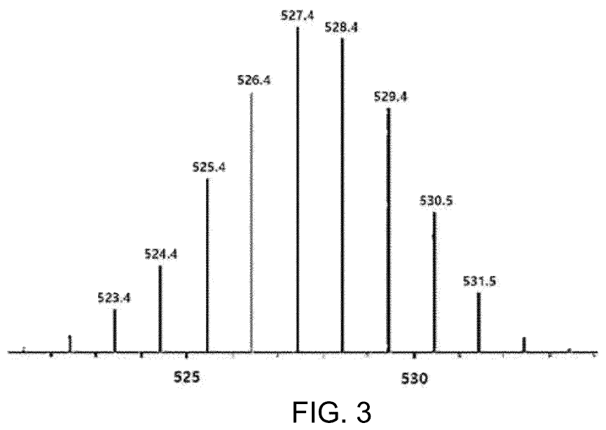

For example, the structure of the organic light emitting device according to an exemplary embodiment of the present specification is exemplified in FIGS. 1 and 2. FIGS. 1 and 2 exemplify an organic light emitting device, and the organic light emitting device is not limited thereto.

FIG. 1 exemplifies a structure of an organic light emitting device in which a first electrode 2, a light emitting layer 4, and a second electrode 3 are sequentially stacked on a substrate 1. The compound is included in the light emitting layer.

FIG. 2 exemplifies the structure of an organic light emitting device in which a first electrode 2, a hole injection layer 5, a hole transport layer 6, a hole adjusting layer 7, a light emitting layer 4, an electron adjusting layer 8, an electron transport layer 9, an electron injection layer 10, a second electrode 3, and a capping layer 11 are sequentially stacked on a substrate 1. The compound is included in the light emitting layer.

The organic light emitting device of the present specification may be manufactured by the materials and methods known in the art, except that the light emitting layer is included or the light emitting layer includes the compound, that is, the compound of Chemical Formula 1.

When the organic light emitting device includes a plurality of organic material layers, the organic material layers may be formed with materials the same as or different from each other.

For example, the organic light emitting device of the present specification may be manufactured by sequentially stacking a first electrode, an organic material layer, and a second electrode on a substrate. In this case, the organic light emitting device may be manufactured by depositing a metal or a metal oxide having conductivity, or an alloy thereof on a substrate to form an anode, forming an organic material layer including a hole injection layer, a hole transport layer, a light emitting layer, and an electron transport layer thereon, and then depositing a material, which may be used as a cathode, thereon, by using a physical vapor deposition (PVD) method such as sputtering or e-beam evaporation. In addition to the method described above, an organic light emitting device may be manufactured by sequentially depositing a second electrode material, an organic material layer, and a first electrode material on a substrate.

Further, the compound represented by Chemical Formula 1 may be formed as an organic material layer by not only a vacuum deposition method, but also a solution application method when an organic light emitting device is manufactured. Here, the solution application method means spin coating, dip coating, doctor blading, inkjet printing, screen printing, a spray method, roll coating, and the like, but is not limited thereto.

In addition to the method described above, an organic light emitting device may be made by sequentially depositing a second electrode material, an organic material layer, and a first electrode material on a substrate. However, the manufacturing method is not limited thereto.

As the first electrode material, materials having a high work function are usually preferred so as to facilitate the injection of holes into an organic material layer. Examples thereof include: a metal, such as vanadium, chromium, copper, zinc, and gold, or an alloy thereof; a metal oxide, such as zinc oxide, indium oxide, indium tin oxide (ITO), and indium zinc oxide (IZO); a combination of a metal and an oxide, such as ZnO:Al or SnO₂:Sb; a conductive polymer, such as poly(3-methylthiophene), poly[3,4-(ethylene-1,2-dioxy)thiophene](PEDOT), polypyrrole, and polyaniline; and the like, but are not limited thereto.

As the second electrode material, materials having a low work function are usually preferred so as to facilitate the injection of electrons into an organic material layer. Examples thereof include: a metal, such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

The light emitting layer may include a host material and a dopant material. When an additional light emitting layer is included in addition to a light emitting layer including the compound of Chemical Formula 1 according to an exemplary embodiment of the present specification, examples of a host material include a fused and/or non-fused aromatic ring derivative, a hetero ring-containing compound, or the like. Specific examples of the fused aromatic ring derivative include anthracene derivatives, pyrene derivatives, naphthalene derivatives, pentacene derivatives, phenanthrene compounds, fluoranthene compounds, and the like, and specific examples of the hetero ring-containing compound include dibenzofuran derivatives, ladder-type furan compounds, pyrimidine derivatives, and the like, but the examples are not limited thereto.

According to an exemplary embodiment of the present specification, the host includes the compound represented by Chemical Formula 2, but is not limited thereto.

Examples of the dopant material include an aromatic amine derivative, a styrylamine compound, a boron complex, a fluoranthene compound, a metal complex, and the like. Specifically, the aromatic amine derivative is a fused aromatic ring derivative having a substituted or unsubstituted arylamine group, and examples thereof include pyrene, anthracene, chrysene, periflanthene, and the like having an arylamine group. Further, the styrylamine compound is a compound in which a substituted or unsubstituted arylamine is substituted with at least one arylvinyl group, and one or two or more substituents selected from the group consisting of an aryl group, a silyl group, an alkyl group, a cycloalkyl group, and an arylamine group are substituted or unsubstituted. Specific examples thereof include styrylamine, styryldiamine, styryltriamine, styryltetramine, and the like, but are not limited thereto. Further, examples of the metal complex include an iridium complex, a platinum complex, and the like, but are not limited thereto.

According to an exemplary embodiment of the present specification, the dopant material includes a compound of the following Chemical Formula D-1 or D-2, but is not limited thereto.

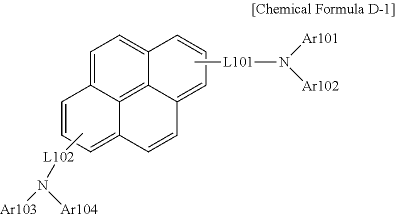

In Chemical Formula D-1,
L101 and L102 are the same as or different from each other, and are each independently a direct bond; or a substituted or unsubstituted arylene group, and
Ar101 to Ar104 are the same as or different from each other, and are each independently a substituted or unsubstituted aryl group; or a substituted or unsubstituted heteroaryl group,

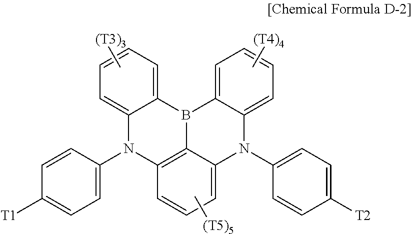

In Chemical Formula D-2,
T1 to T5 are the same as or different from each other, and are each independently hydrogen; a substituted or unsubstituted alkyl group; a substituted or unsubstituted amine group; or a substituted or unsubstituted aryl group,
t3 and t4 are each an integer from 1 to 4,
t5 is an integer from 1 to 3,
when t3 is 2 or higher, two or more T3's are the same as or different from each other,
when t4 is 2 or higher, two or more T4's are the same as or different from each other, and
when t5 is 2 or higher, two or more T5's are the same as or different from each other.

According to an exemplary embodiment of the present specification, L101 and L102 are a direct bond.

According to an exemplary embodiment of the present specification, Ar101 to Ar104 are the same as or different from each other, and are each independently a substituted or unsubstituted monocyclic or polycyclic aryl group having 6 to 30 carbon atoms; or a substituted or unsubstituted monocyclic or polycyclic heteroaryl group having 2 to 30 carbon atoms.

According to an exemplary embodiment of the present specification, Ar101 to Ar104 are the same as or different from each other, and are each independently a monocyclic or polycyclic aryl group having 6 to 30 carbon atoms, which is unsubstituted or substituted with a straight-chained or branched alkyl group having 1 to 30 carbon atoms; or a monocyclic or polycyclic heteroaryl group having 2 to 30 carbon atoms.

According to an exemplary embodiment of the present specification, Ar101 to Ar104 are the same as or different from each other, and are each independently a phenyl group substituted with a methyl group; or a dibenzofuran group.

According to an exemplary embodiment of the present specification, Chemical Formula D-1 is represented by the following compound.

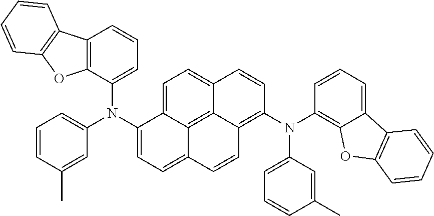 [SMILES: Cc1cccc(N(c2ccc3ccc4c(N(c5cccc(C)c5)c5cccc6c5oc5ccccc56)ccc5ccc2c3c54)c2cccc3c2oc2ccccc23)c1]

According to an exemplary embodiment of the present specification, T1 to T5 are the same as or different from each other, and are each independently hydrogen; a substituted or unsubstituted straight-chained or branched alkyl group having 1 to 30 carbon atoms; a substituted or unsubstituted monocyclic or polycyclic arylamine group having 6 to 30 carbon atoms; or a substituted or unsubstituted monocyclic or polycyclic aryl group having 6 to 30 carbon atoms.

According to an exemplary embodiment of the present specification, T1 to T5 are the same as or different from each other, and are each independently hydrogen; a straight-chained or branched alkyl group having 1 to 30 carbon atoms; a monocyclic or polycyclic arylamine group having 6 to 30 carbon atoms; or a monocyclic or polycyclic aryl group having 6 to 30 carbon atoms, which is unsubstituted or substituted with a straight-chained or branched alkyl group having 1 to 30 carbon atoms.

According to an exemplary embodiment of the present specification, T1 to T5 are the same as or different from each other, and are each independently hydrogen; a methyl group; a tert-butyl group; a diphenylamine group; or a phenyl group which is unsubstituted or substituted with a methyl group or tert-butyl group.

According to an exemplary embodiment of the present specification, Chemical Formula D-2 is represented by the following compound.

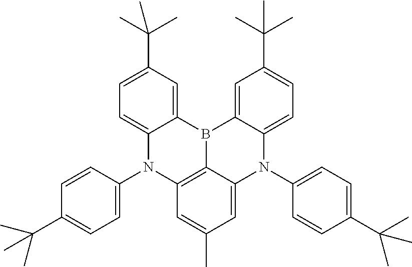 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

The hole injection layer is a layer which accepts holes from an electrode. It is preferred that the hole injection material has an ability to transport holes, and has an effect of accepting holes from an anode and an excellent hole injection effect for a light emitting layer or a light emitting material. Further, the hole injection material is preferably a material which is excellent in ability to prevent excitons produced from a light emitting layer from moving to an electron injection layer or an electron injection material. In addition, the hole injection material is preferably a material which is excellent in ability to form a thin film. In addition, the highest occupied molecular orbital (HOMO) of the hole injection material is preferably a value between the work function of the anode material and the HOMO of the neighboring organic material layer. Specific examples of the hole injection material include: metal porphyrin, oligothiophene, and arylamine-based organic materials; hexanitrile hexaazatriphenylene-based organic materials; quinacridone-based organic materials; perylene-based organic materials; polythiophene-based conductive polymers such as anthraquinone and polyaniline; and the like, but are not limited thereto.

The hole injection layer is a layer which accepts holes from an electrode. It is preferred that the hole injection material has an ability to transport holes, and has an effect of accepting holes from an anode and an excellent hole injection effect for a light emitting layer or a light emitting material. Further, the hole injection material is preferably a material which is excellent in ability to prevent excitons produced from a light emitting layer from moving to an electron injection layer or an electron injection material. In addition, the hole injection material is preferably a material which is excellent in ability to form a thin film. In addition, the highest occupied molecular orbital (HOMO) of the hole injection material is preferably a value between the work function of the anode material and the HOMO of the neighboring organic material layer. Specific examples of the hole injection material include: metal porphyrin, oligothiophene, and arylamine-based organic materials; hexanitrile hexaazatriphenylene-based organic materials; quinacridone-based organic materials; perylene-based organic materials; polythiophene-based conductive polymers such as anthraquinone and polyaniline; and the like, but are not limited thereto.

According to an exemplary embodiment of the present specification, the hole injection layer includes a compound represented by the following Chemical Formula HI-1, but is not limited thereto.

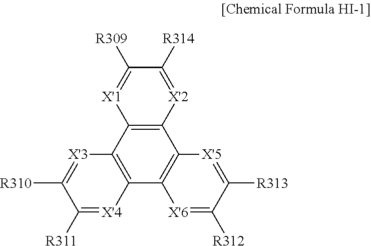

In Chemical Formula HI-1,
at least one of X′1 to X′6 is N, and the others are CH, and
R309 to R314 are the same as or different from each other, and are each independently hydrogen; deuterium; a cyano group; a substituted or unsubstituted alkyl group; a substituted or unsubstituted amine group; a substituted or unsubstituted aryl group; or a substituted or unsubstituted heteroaryl group, or are bonded to an adjacent group to form a substituted or unsubstituted ring.

According to an exemplary embodiment of the present specification, X′1 to X′6 are N.

According to an exemplary embodiment of the present specification, R309 to R314 are a cyano group.

According to an exemplary embodiment of the present specification, Chemical Formula HI-1 is represented by the following compound.

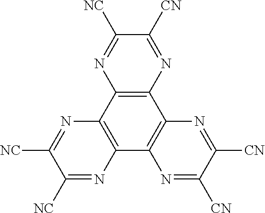 [SMILES: N#Cc1nc2c3nc(C#N)c(C#N)nc3c3nc(C#N)c(C#N)nc3c2nc1C#N]

The hole transport layer is a layer which accepts holes from a hole injection layer and transports the holes to a light emitting layer. A hole transport material is preferably a material having high hole mobility which may accept holes from an anode or a hole injection layer and transfer the holes to a light emitting layer. Specific examples thereof include arylamine-based organic materials, conductive polymers, block copolymers having both conjugated portions and non-conjugated portions, and the like, but are not limited thereto.

According to an exemplary embodiment of the present specification, the hole transport layer or hole adjusting layer includes a compound of following Chemical Formula HT-1, but is not limited thereto.

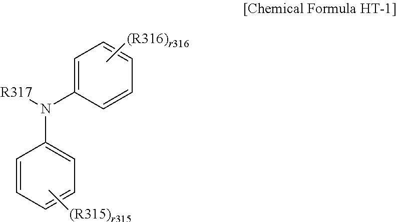

In Chemical Formula HT-1,
R315 to R317 are the same as or different from each other, and are each independently any one selected from the group consisting of hydrogen; deuterium; a substituted or unsubstituted alkyl group; a substituted or unsubstituted aryl group; a substituted or unsubstituted heteroaryl group; and a combination thereof, or are bonded to an adjacent group to form a substituted or unsubstituted ring,
r315 is an integer from 1 to 5, and when r315 is 2 or higher, two or more R315's are the same as or different from each other, and
r316 is an integer from 1 to 5, and when r316 is 2 or higher, two or more R316's are the same as or different from each other.

According to an exemplary embodiment of the present specification, R317 is any one selected from the group consisting of a substituted or unsubstituted aryl group; a substituted or unsubstituted heteroaryl group; and a combination thereof.

According to an exemplary embodiment of the present specification, R317 is any one selected from the group consisting of a carbazole group; a phenyl group; a biphenyl group; a fluorene group; and a combination thereof.

According to an exemplary embodiment of the present specification, R315 and R316 are the same as or different from each other, and are each independently a substituted or unsubstituted aryl group, or are bonded to an adjacent group to form an aromatic hydrocarbon ring substituted with an alkyl group.

According to an exemplary embodiment of the present specification, R315 and R316 are the same as or different from each other, and are each independently a phenyl group or a phenanthrene group, or are bonded to an adjacent group to form indene substituted with a methyl group.

According to an exemplary embodiment of the present specification, Chemical Formula HT-1 is represented by any one of the following compounds.

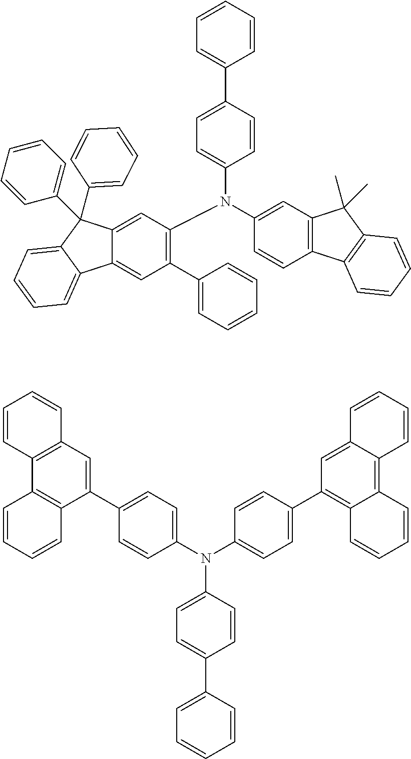 [SMILES: CC1(C)c2ccccc2-c2ccc(N(c3ccc(-c4ccccc4)cc3)c3cc4c(cc3-c3ccccc3)-c3ccccc3C4(c3ccccc3)c3ccccc3)cc21 ; c1ccc(-c2ccc(N(c3ccc(-c4cc5ccccc5c5ccccc45)cc3)c3ccc(-c4cc5ccccc5c5ccccc45)cc3)cc2)cc1]

The hole adjusting layer is a layer which may improve the service life and efficiency of a device by adjusting holes transported from a hole transport layer such that the holes are smoothly injected into a light emitting layer, and preventing electrons injected from an electron injection layer from passing through a light emitting layer and entering a hole injection layer. As the hole adjusting layer, the publicly-known material can be used without limitation, and the hole adjusting layer may be formed between a light emitting layer and a hole injection layer, between a light emitting layer and a hole transport layer, or between a light emitting layer and a layer which simultaneously injects and transports holes.

As a material for the electron adjusting layer, the examples of Chemical Formula HI-I may be applied, but the material is not limited thereto.

The electron adjusting layer is a layer that adjusts electrons transferred from the electron transport layer to be smoothly injected into the light emitting layer, and known materials can be used without limitation.

According to an exemplary embodiment of the present specification, the electron adjusting layer includes a compound of the following Chemical Formula EG-1, but is not limited thereto.

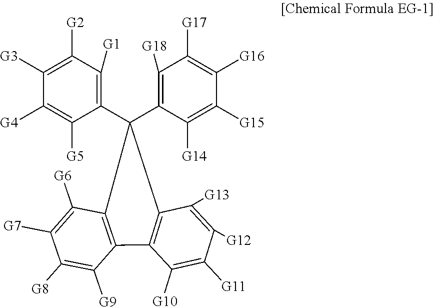

In Chemical Formula EG-1,
at least one of G1 to G18 is -L5-Ar5, the others are hydrogen, or G1 and G18 are linked through -L51- to form a substituted or unsubstituted ring,
L5 is a direct bond; or a substituted or unsubstituted arylene group,
Ar5 is a substituted or unsubstituted aryl group; or a substituted or unsubstituted heteroaryl group, and L51 is 0; or S.

According to an exemplary embodiment of the present specification, L51 is 0.

According to an exemplary embodiment of the present specification, L51 is S.

According to an exemplary embodiment of the present specification, G1 and G18 are linked through -L51- to form a substituted or unsubstituted hetero ring.

According to an exemplary embodiment of the present specification, G1 and G18 are linked through -L51- to form a substituted or unsubstituted xanthene ring; or a substituted or unsubstituted thioxanthene ring.

According to an exemplary embodiment of the present specification, G1 and G18 are linked through —O— to form a substituted or unsubstituted xanthene ring.

According to an exemplary embodiment of the present specification, G1 and G18 are linked through —S— to form a substituted or unsubstituted thioxanthene ring.

According to an exemplary embodiment of the present specification, G1 and G18 are linked through —O— to form a xanthene ring.

According to an exemplary embodiment of the present specification, G1 and G18 are linked through —S— to form a thioxanthene ring.

According to an exemplary embodiment of the present specification, L5 is a direct bond; or a substituted or unsubstituted monocyclic or polycyclic arylene group having 6 to 30 carbon atoms.

According to an exemplary embodiment of the present specification, L5 is a direct bond; or a substituted or unsubstituted monocyclic or polycyclic arylene group having 6 to 20 carbon atoms.

According to an exemplary embodiment of the present specification, L5 is a direct bond; or a monocyclic or polycyclic arylene group having 6 to 30 carbon atoms.

According to an exemplary embodiment of the present specification, L5 is a direct bond; or a monocyclic or polycyclic arylene group having 6 to 20 carbon atoms.

According to an exemplary embodiment of the present specification, L5 is a direct bond; or a phenylene group.

According to an exemplary embodiment of the present specification, Ar5 is a substituted or unsubstituted triazine group.

According to an exemplary embodiment of the present specification, Ar5 is a triazine group which is unsubstituted or substituted with a monocyclic or polycyclic aryl group having 6 to 30 carbon atoms.

According to an exemplary embodiment of the present specification, Ar5 is a triazine group substituted with a phenyl group.

According to an exemplary embodiment of the present specification, EG-1 is represented by the following compound.

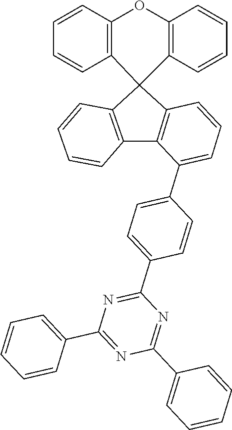 [SMILES: c1ccc(-c2nc(-c3ccccc3)nc(-c3ccc(-c4cccc5c4-c4ccccc4C54c5ccccc5Oc5ccccc54)cc3)n2)cc1]

The electron transport layer is a layer which accepts electrons from an electron injection layer and transports the electrons to a light emitting layer. An electron transport material is preferably a material having high electron mobility which may proficiently accept electrons from a cathode and transfer the electrons to a light emitting layer. Specific examples thereof include: Al complexes of 8-hydroxyquinoline; complexes including Alq3; organic radical compounds; hydroxyflavone-metal complexes; and the like, but are not limited thereto. The electron transport layer may be used with any desired cathode material, as used according to the related art. In particular, an appropriate cathode material is a typical material which has a low work function, followed by an aluminum layer or a silver layer. Specific examples thereof include cesium, barium, calcium, ytterbium, and samarium, in each case followed by an aluminum layer or a silver layer.

According to an exemplary embodiment of the present specification, the electron transport layer includes a compound of the following Chemical Formula ET-1, but is not limited thereto.

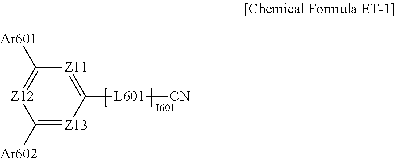

In Chemical Formula ET-1,
at least one of Z11 to Z13 is N, and the others are CH,
L601 is a direct bond; a substituted or unsubstituted arylene group; or a substituted or unsubstituted heteroarylene group,
Ar601 and Ar602 are the same as or different from each other, and are each independently a substituted or unsubstituted aryl group; or a substituted or unsubstituted heteroaryl group, and
1601 is an integer from 1 to 5, and when 1601 is 2 or higher, two or more L601's are the same as or different from each other.

According to an exemplary embodiment of the present specification, L601 is a substituted or unsubstituted monocyclic or polycyclic arylene group having 6 to 30 carbon atoms.

According to an exemplary embodiment of the present specification, L601 is a phenylene group; a biphenylylene group; or a naphthylene group.

According to an exemplary embodiment of the present specification, Ar601 and Ar602 are the same as or different from each other, and are each independently a substituted or unsubstituted monocyclic or polycyclic aryl group having 6 to 30 carbon atoms.

According to an exemplary embodiment of the present specification, Ar601 and Ar602 are a phenyl group.

According to an exemplary embodiment of the present specification, Chemical Formula ET-1 is represented by the following compound.

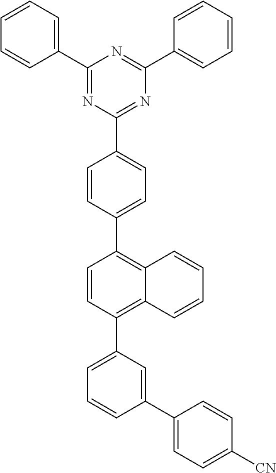 [SMILES: N#Cc1ccc(-c2cccc(-c3ccc(-c4ccc(-c5nc(-c6ccccc6)nc(-c6ccccc6)n5)cc4)c4ccccc34)c2)cc1]

The electron injection layer is a layer which accepts electrons from an electrode. It is preferred that an electron injection material is excellent in ability to transport electrons and has an effect of accepting electrons from the second electrode and an excellent electron injection effect for a light emitting layer or a light emitting material. Further, the electron injection material is preferably a material which prevents excitons produced from a light emitting layer from moving to a hole injection layer and is excellent in ability to form a thin film. Specific examples thereof include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, metal complex compounds, nitrogen-containing 5-membered ring derivatives, and the like, but are not limited thereto.

Examples of the metal complex compounds include 8-hydroxyquinolinato lithium, bis(8-hydroxyquinolinato) zinc, bis(8-hydroxyquinolinato) copper, bis(8-hydroxyquinolinato) manganese, tris(8-hydroxyquinolinato) aluminum, tris(2-methyl-8-hydroxyquinolinato) aluminum, tris(8-hydroxyquinolinato) gallium, bis(10-hydroxybenzo[h]quinolinato) beryllium, bis(10-hydroxybenzo[h]quinolinato) zinc, bis(2-methyl-8-quinolinato) chlorogallium, bis(2-methyl-8-quinolinato) (o-cresolato) gallium, bis(2-methyl-8-quinolinato) (1-naphtholato) aluminum, bis(2-methyl-8-quinolinato) (2-naphtholato) gallium, and the like, but are not limited thereto.

According to an exemplary embodiment of the present specification, the electron injection and transport layer is a layer that transports electrons to the light emitting layer. For the electron injection and transport layer, materials exemplified for the electron transport layer and the electron injection layer may be used, but the materials are not limited thereto.

According to an exemplary embodiment of the present specification, the electron injection and transport layer may further include a metal complex compound. The metal complex compound is as described above.

The electron adjusting layer is a layer which may improve the service life and efficiency of a device by preventing electrons injected from an electron injection layer from passing through a light emitting layer and entering a hole injection layer. The publicly-known material can be used without limitation, and the electron adjusting layer may be formed between a light emitting layer and a hole injection layer, between a light emitting layer and a hole transport layer, or between a light emitting layer and a layer which simultaneously injects and transports holes.

As a material for the electron adjusting layer, the examples of Chemical Formula HI-I may be applied, but the material is not limited thereto.

The hole adjusting layer is a layer which blocks holes from reaching a cathode, and may be generally formed under the same conditions as those of the electron injection layer. Specific examples thereof include oxadiazole derivatives or triazole derivatives, phenanthroline derivatives, aluminum complexes, and the like, but are not limited thereto.

The capping layer is formed to prevent a considerable amount of light from being lost through total reflection of light in the organic light emitting device, and the capping layer has the performance capable of sufficiently protecting the underlying negative electrode and light emitting layer from external moisture penetration or contamination, and has a high refractive index, and thus may prevent light loss caused by total reflection, and materials in the related art can be used without limitation.

According to an exemplary embodiment of the present specification, the capping layer includes a compound represented by the following Chemical Formula CP-1, but is not limited thereto.

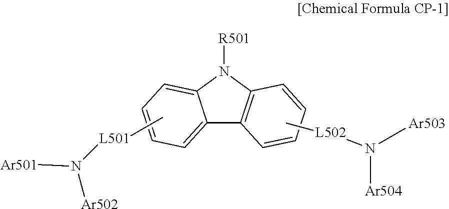

In Chemical Formula CP-1,
L501 and L502 are the same as or different from each other, and are each independently a direct bond; a substituted or unsubstituted arylene group; or a substituted or unsubstituted heteroarylene group, and
R501 and Ar501 to Ar504 are the same as or different from each other, and are each independently a substituted or unsubstituted aryl group; or a substituted or unsubstituted heteroaryl group, or adjacent groups are bonded to each other to form a substituted or unsubstituted ring.

According to an exemplary embodiment of the present specification, L501 and L502 are the same as or different from each other, and are each independently a substituted or unsubstituted monocyclic or polycyclic arylene group having 6 to 30 carbon atoms.

According to an exemplary embodiment of the present specification, L501 and L502 are a phenylene group.

According to an exemplary embodiment of the present specification, R501 and Ar501 to Ar504 are the same as or different from each other, and are each independently a substituted or unsubstituted monocyclic or polycyclic aryl group having 6 to 30 carbon atoms, or are bonded to an adjacent group to form a substituted or unsubstituted monocyclic or polycyclic hetero ring having 2 to 30 carbon atoms.

According to an exemplary embodiment of the present specification, R501 and Ar501 to Ar504 are a phenyl group, or are bonded to an adjacent group to form a carbazole which is unsubstituted or substituted with a phenyl group.

According to an exemplary embodiment of the present specification, Ar501 and L501 are bonded to form a carbazole substituted with a phenyl group.

According to an exemplary embodiment of the present specification, Ar503 is bonded to L503 to form a carbazole substituted with a phenyl group.

According to an exemplary embodiment of the present specification, Chemical Formula CP-1 is represented by the following compound.

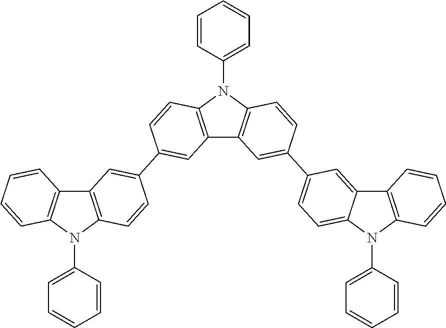 [SMILES: c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1]

The organic light emitting device according to the present specification may be a top-emission type, a bottom-emission type or a dual-emission type depending on the materials used.

The organic light emitting device according to the present specification may be included and used in various electronic devices. For example, the electronic device may be a display panel, a touch panel, a solar module, a lighting device, and the like, and is not limited thereto.

Hereinafter, the present specification will be described in detail with reference to Examples for specifically describing the present specification. However, the Examples according to the present specification may be modified in various forms, and it is not interpreted that the scope of the present application is limited to the Examples described in detail below. The Examples of the present application are provided to explain the present specification more completely to a person with ordinary skill in the art.

### Preparation Example 1 (Synthesis of Chemical Formula 1)

### 1) Synthesis of Chemical Formulae A1 and B1

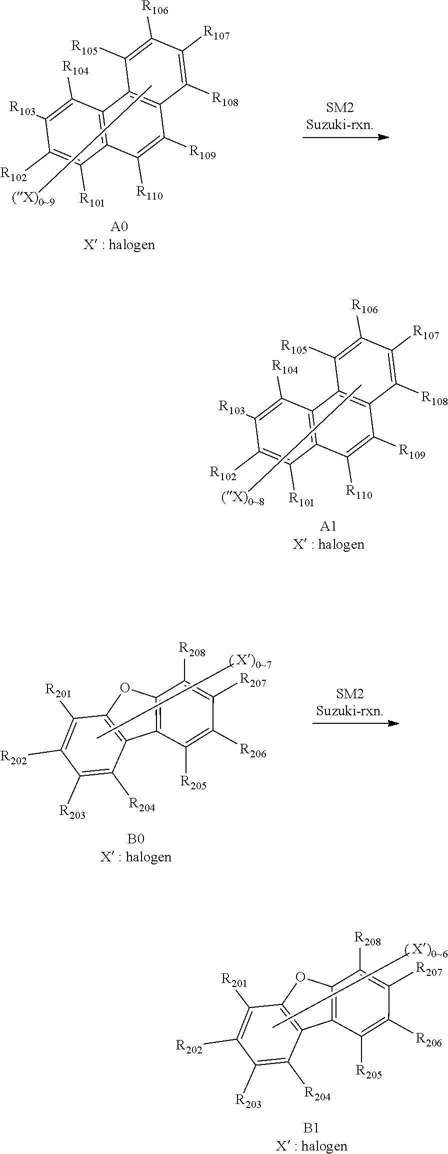

After SM1 (one of A0 or B0, 1 eq) and SM2 (1.1 eq) were added to tetrahydrofuran (THF) ° C. (excess), a 2 M aqueous potassium carbonate solution (30 volume ratio compared to THF) was added thereto, and tetrakis triphenyl-phosphino palladium (2 mol %) was added thereto, and then the resulting mixture was heated and stirred at 85° C. for 10 hours. After the temperature was lowered to room temperature and the reaction was terminated, layers were separated by removing the aqueous potassium carbonate solution, and the residue was columned with hexane and ethyl acetate to prepare Chemical Formulae A1 and B1 (A1-1 to A1-6 and B1-1 to B1-3), respectively.

A1-1 to A1-6 in Table 1 and B1-1 to B1-3 in Table 2 were synthesized in the same manner as in the method of synthesizing Chemical Formulae A1 and B1, except that SM1 and SM2 were changed to the materials in the following Tables 1 and 2.

TABLE 1

| | | | | | MS |
|---|---|---|---|---|---|
| | SM1 | SM2 | Product | Yield | [M + H]+ |
| A1-1 | 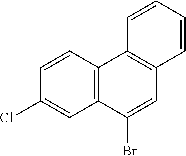 | 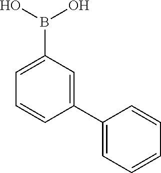 | 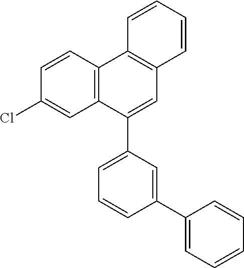 [SMILES: Clc1ccc2c(c1)c(-c1cccc(-c3ccccc3)c1)cc1ccccc12] | 73% | 365.87 |
| A1-2 | 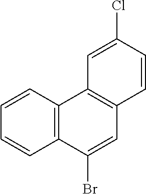 | 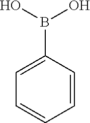 | 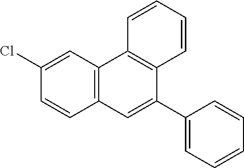 [SMILES: Clc1ccc2cc(-c3ccccc3)c3ccccc3c2c1] | 75% | 289.77 |
| A1-3 | 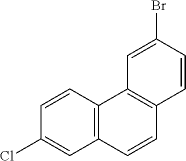 | 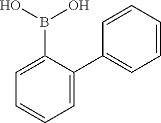 | 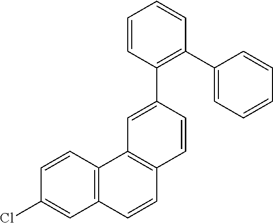 [SMILES: Clc1ccc2c(ccc3ccc(-c4ccccc4-c4ccccc4)cc32)c1] | 70% | 365.87 |
| A1-4 | 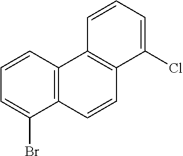 | 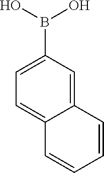 | 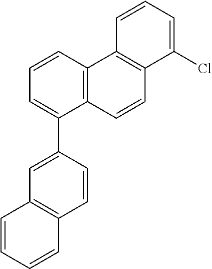 [SMILES: Clc1cccc2c1ccc1c(-c3ccc4ccccc4c3)cccc12] | 71% | 339.83 |
| A1-5 | 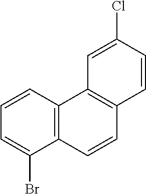 | 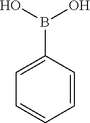 | 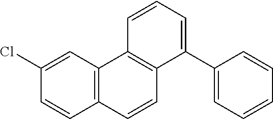 [SMILES: Clc1ccc2ccc3c(-c4ccccc4)cccc3c2c1] | 69% | 289.77 |
| A1-6 | 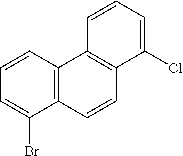 | 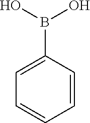 | 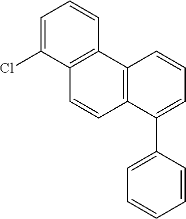 [SMILES: Clc1cccc2c1ccc1c(-c3ccccc3)cccc12] | 65% | 289.77 |

TABLE 2

| | | | | | MS |
|---|---|---|---|---|---|
| | SM1 | SM2 | Product | Yield | [M + H]+ |
| B1-1 | 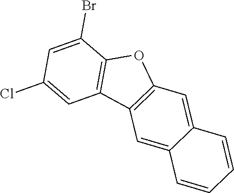 | 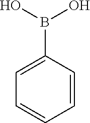 | 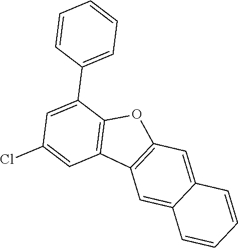 [SMILES: Clc1cc(-c2ccccc2)c2oc3cc4ccccc4cc3c2c1] | 75% | 329.80 |
| B1-2 | 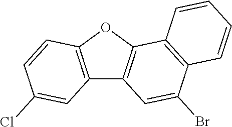 | 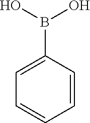 | 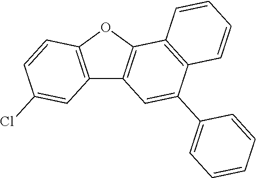 [SMILES: Clc1ccc2oc3c4ccccc4c(-c4ccccc4)cc3c2c1] | 73% | 329.80 |
| B1-3 | 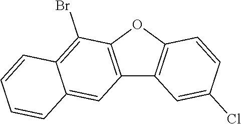 | 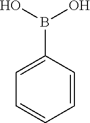 | 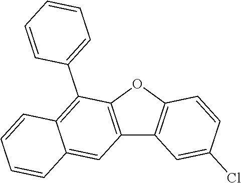 [SMILES: Clc1ccc2oc3c(-c4ccccc4)c4ccccc4cc3c2c1] | 74% | 329.80 |

### 2) Synthesis of Chemical Formulae A2 and B2

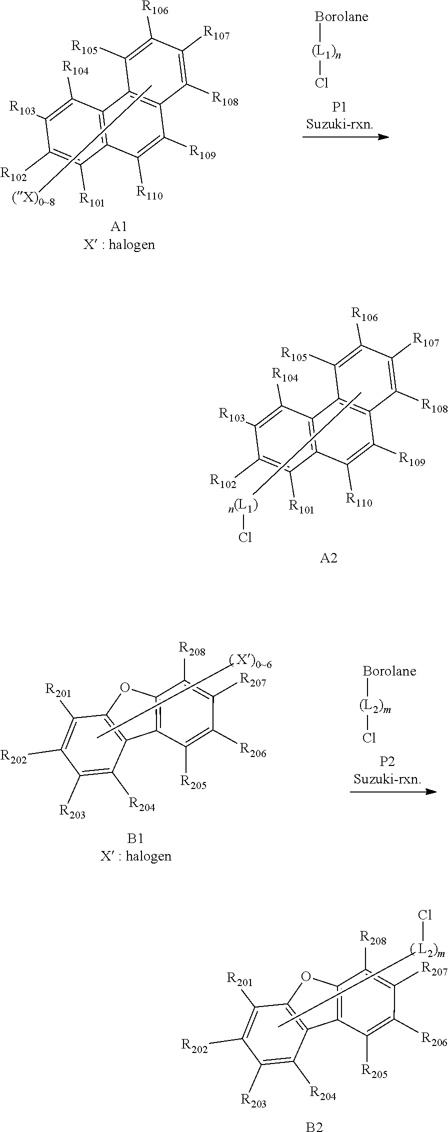

After SM1 (one of A1 or B1, 1 eq) and SM2 (one of P1 or P2, 1.1 eq) were added to tetrahydrofuran (THF) (excess), a 2 M aqueous potassium carbonate solution (30 volume ratio compared to THF) was added thereto, and tetrakis triphenyl-phosphino palladium (2 mol %) was added thereto, and then the resulting mixture was heated and stirred at 85° C. for 10 hours. After the temperature was lowered to room temperature and the reaction was terminated, layers were separated by removing the aqueous potassium carbonate solution, and the residue was columned with hexane and ethyl acetate to prepare Chemical Formulae A2 and B2 (A2-1 to A2-4 and B2- to B2-2), respectively.

A2-1 to A2-4 in Table 3 and B2-1 to B2-2 in Table 4 were synthesized in the same manner as in the method of synthesizing Chemical Formulae A2 and B2, except that SM1 and SM2 were changed to the materials in the following Tables 3 and 4.

TABLE 3

| | | | | | MS |
|---|---|---|---|---|---|
| | SM1 | SM2 | Product | Yield | [M + H]+ |
| A2-1 | 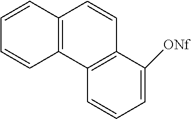 | 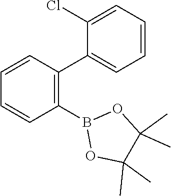 | 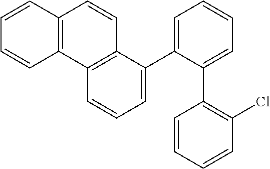 [SMILES: Clc1ccccc1-c1ccccc1-c1cccc2c1ccc1ccccc12] | 73% | 365.87 |
| A2-2 | 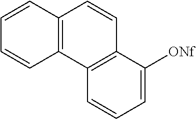 | 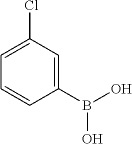 | 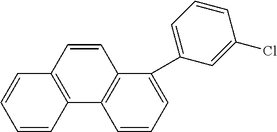 [SMILES: Clc1cccc(-c2cccc3c2ccc2ccccc23)c1] | 72% | 289.77 |
| A2-3 | 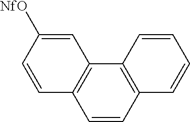 | 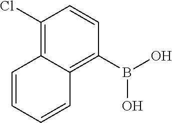 | 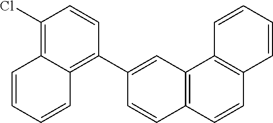 [SMILES: Clc1ccc(-c2ccc3ccc4ccccc4c3c2)c2ccccc12] | 70% | 339.83 |
| A2-4 | 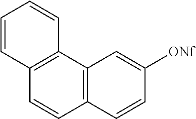 | 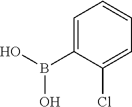 | 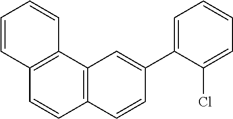 [SMILES: Clc1ccccc1-c1ccc2ccc3ccccc3c2c1] | 65% | 289.77 |

TABLE 4

| | | | | | MS |
|---|---|---|---|---|---|
| | SM1 | SM2 | Product | Yield | [M + H]+ |
| B2-1 | 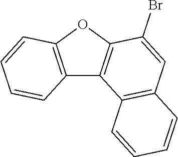 | 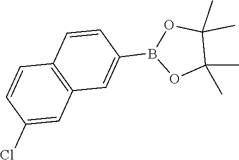 | 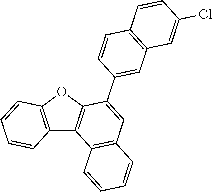 [SMILES: Clc1ccc2ccc(-c3cc4ccccc4c4c3oc3ccccc34)cc2c1] | 70% | 379.86 |
| B2-2 | 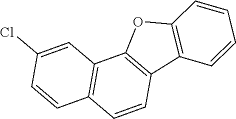 | 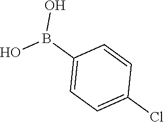 | 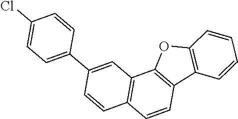 [SMILES: Clc1ccc(-c2ccc3ccc4c5ccccc5oc4c3c2)cc1] | 74% | 329.80 |

### 3) Synthesis of Chemical Formulae A3 and B3

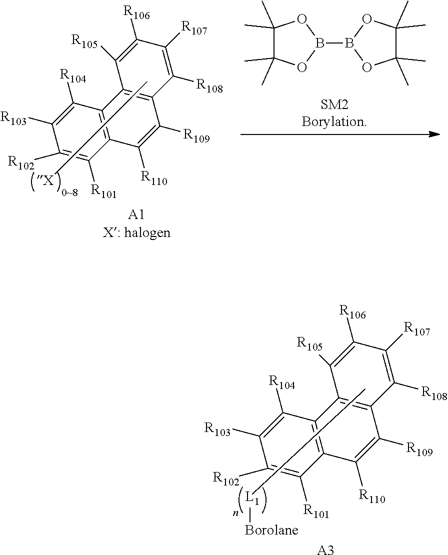

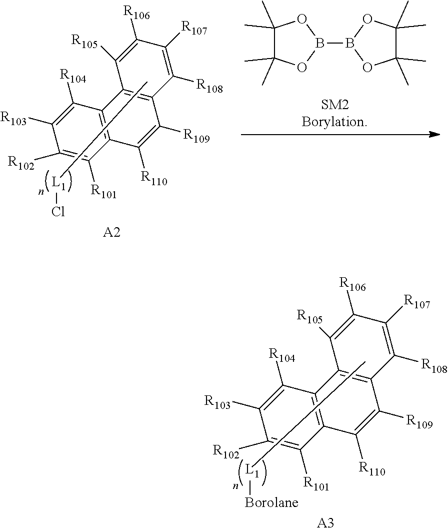

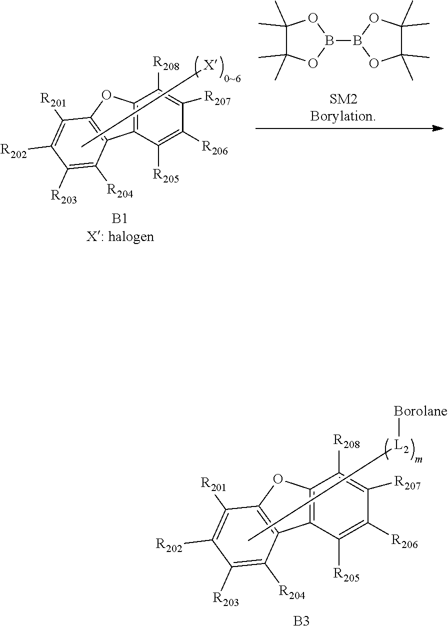

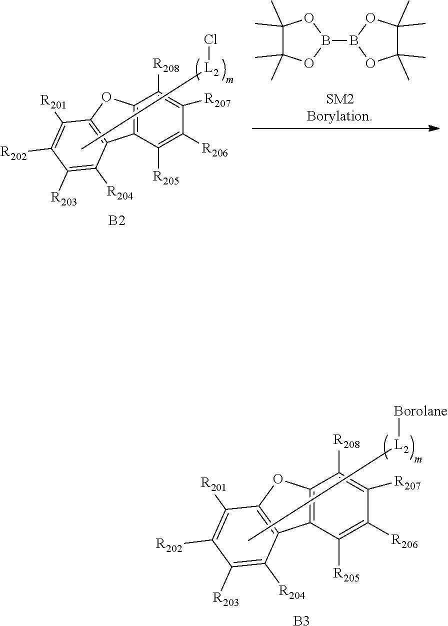

SM1 (one of A1 and A2 or B1 and B2, 1 eq) and SM2 (1.3 eq) were put into 1,4-dioxane (12-fold <mass ratio> compared to SM1), and potassium acetate (3 eq) was added thereto, and the resulting mixture was stirred and refluxed. After bis(diphenylphosphino)ferrocene dichloropalladium (0.05 eq) was stirred in 1,4-dioxane for 5 minutes, the resulting mixture was added thereto, and 2 hours later, the mixture was cooled to room temperature after confirming that the reaction was terminated. After ethanol and water were added thereto, the resulting mixture was filtered and purified by recrystallization with ethyl acetate and ethanol to prepare Chemical Formulae A3 and B3 (A3-1 to A3-9 and B3-1 to B3-27), respectively.

A3-1 to A3-9 in Table 5 and B3-1 to B3-27 in Table 6 were synthesized in the same manner as in the method of synthesizing Chemical Formulae A3 and B3, except that SM1 and SM2 were changed to the materials in the following Tables 5 and 6.

TABLE 5

| | | | | | MS |
|---|---|---|---|---|---|
| | SM1 | SM2 | Product | Yield | [M + H]+ |
| A3-1 | A1-1 | 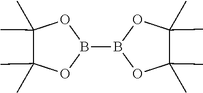 | 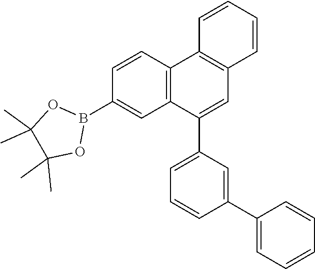 [SMILES: CC1(C)OB(c2ccc3c(c2)c(-c2cccc(-c4ccccc4)c2)cc2ccccc23)OC1(C)C] | 75% | 457.39 |
| A3-2 | A1-2 | 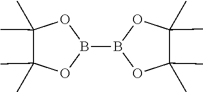 | 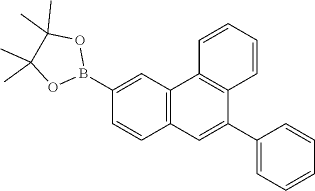 [SMILES: CC1(C)OB(c2ccc3cc(-c4ccccc4)c4ccccc4c3c2)OC1(C)C] | 70% | 381.29 |
| A3-3 | A1-3 | 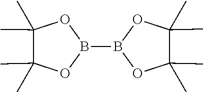 | 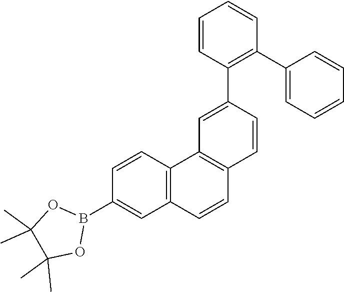 [SMILES: CC1(C)OB(c2ccc3c(ccc4ccc(-c5ccccc5-c5ccccc5)cc43)c2)OC1(C)C] | 76% | 457.39 |
| A3-4 | A1-4 | 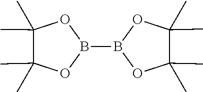 | 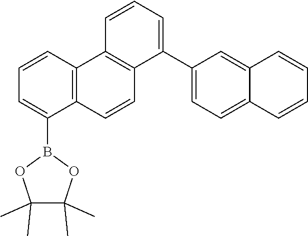 [SMILES: CC1(C)OB(c2cccc3c2ccc2c(-c4ccc5ccccc5c4)cccc23)OC1(C)C] | 73% | 431.35 |
| A3-5 | A2-1 | 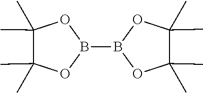 | 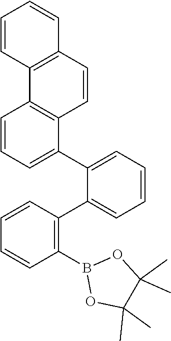 [SMILES: CC1(C)OB(c2ccccc2-c2ccccc2-c2cccc3c2ccc2ccccc23)OC1(C)C] | 74% | 457.39 |
| A3-6 | A2-2 | 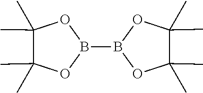 | 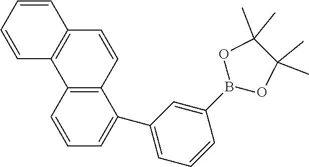 [SMILES: CC1(C)OB(c2cccc(-c3cccc4c3ccc3ccccc34)c2)OC1(C)C] | 71% | 381.29 |
| A3-7 | A2-3 | 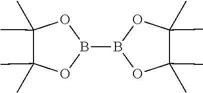 | 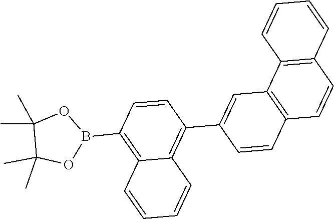 [SMILES: CC1(C)OB(c2ccc(-c3ccc4ccc5ccccc5c4c3)c3ccccc23)OC1(C)C] | 70% | 431.35 |
| A3-8 | A1-5 | 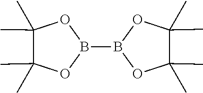 | 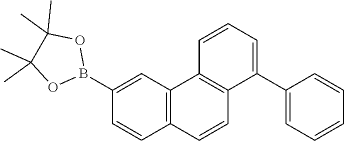 [SMILES: CC1(C)OB(c2ccc3ccc4c(-c5ccccc5)cccc4c3c2)OC1(C)C] | 73% | 381.29 |
| A3-9 | A2-4 | 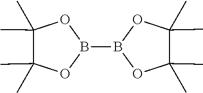 | 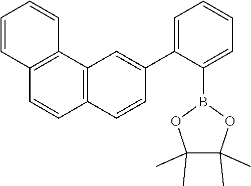 [SMILES: CC1(C)OB(c2ccccc2-c2ccc3ccc4ccccc4c3c2)OC1(C)C] | 70% | 381.29 |

TABLE 6

| | | | | | MS |
|---|---|---|---|---|---|
| | SM1 | SM2 | Product | Yield | [M + H]+ |
| B3-1 | B1-1 | 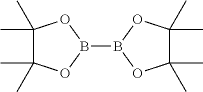 | 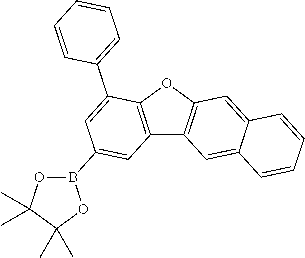 [SMILES: CC1(C)OB(c2cc(-c3ccccc3)c3oc4cc5ccccc5cc4c3c2)OC1(C)C] | 75% | 421.32 |
| B3-2 | 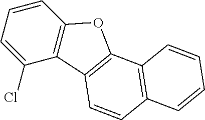 | 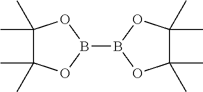 | 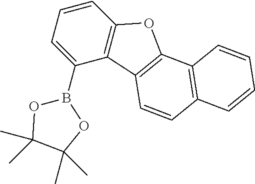 [SMILES: CC1(C)OB(c2cccc3oc4c5ccccc5ccc4c23)OC1(C)C] | 70% | 421.32 |
| B3-3 | 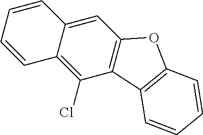 | 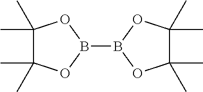 | 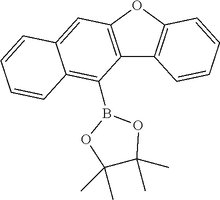 [SMILES: CC1(C)OB(c2c3ccccc3cc3oc4ccccc4c23)OC1(C)C] | 75% | 345.22 |
| B3-4 | 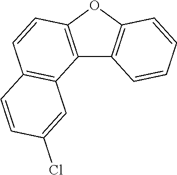 | 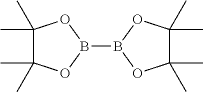 | 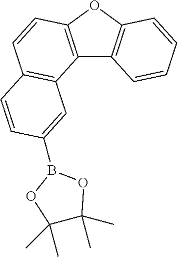 [SMILES: CC1(C)OB(c2ccc3ccc4oc5ccccc5c4c3c2)OC1(C)C] | 76% | 345.22 |
| B3-5 | B1-2 | 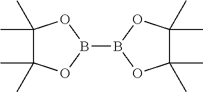 | 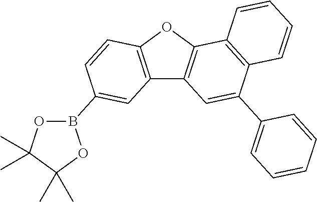 [SMILES: CC1(C)OB(c2ccc3oc4c5ccccc5c(-c5ccccc5)cc4c3c2)OC1(C)C] | 80% | 421.32 |
| B3-6 | 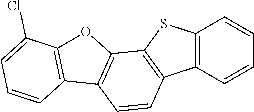 | 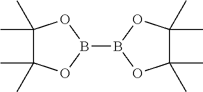 | 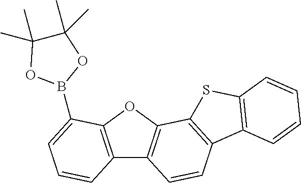 [SMILES: CC1(C)OB(c2cccc3c2oc2c3ccc3c4ccccc4sc32)OC1(C)C] | 77% | 401.30 |
| B3-7 | 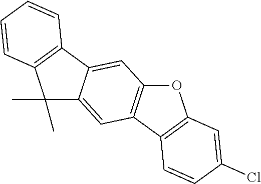 | 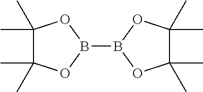 | 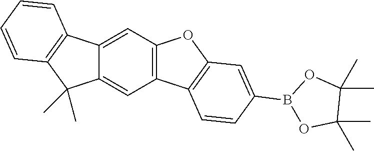 [SMILES: CC1(C)c2ccccc2-c2cc3oc4cc(B5OC(C)(C)C(C)(C)O5)ccc4c3cc21] | 73% | 411.32 |
| B3-8 | 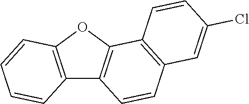 | 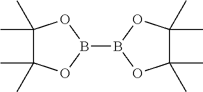 | 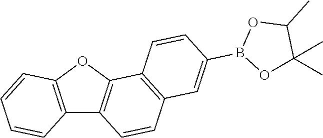 [SMILES: CC1OB(c2ccc3c(ccc4c5ccccc5oc34)c2)OC1(C)C] | 78% | 345.22 |
| B3-9 | B1-3 | 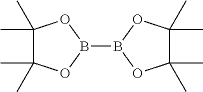 | 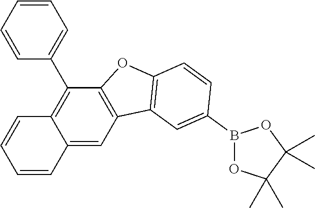 [SMILES: CC1(C)OB(c2ccc3oc4c(-c5ccccc5)c5ccccc5cc4c3c2)OC1(C)C] | 71% | 421.32 |
| B3-10 | 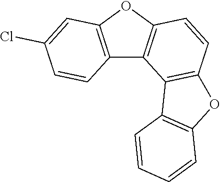 | 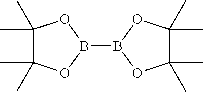 | 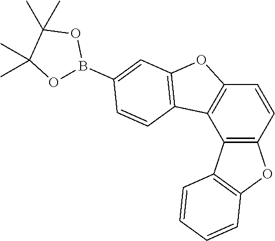 [SMILES: CC1(C)OB(c2ccc3c(c2)oc2ccc4oc5ccccc5c4c23)OC1(C)C] | 70% | 385.24 |
| B3-11 | 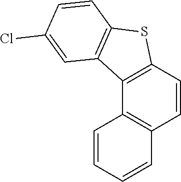 | 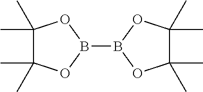 | 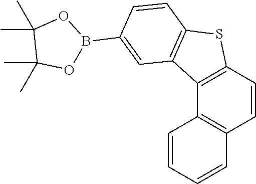 [SMILES: CC1(C)OB(c2ccc3sc4ccc5ccccc5c4c3c2)OC1(C)C] | 73% | 361.28 |
| B3-12 | 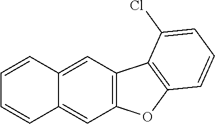 | 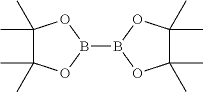 | 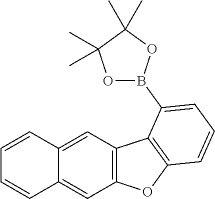 [SMILES: CC1(C)OB(c2cccc3oc4cc5ccccc5cc4c23)OC1(C)C] | 77% | 345.22 |
| B3-13 | 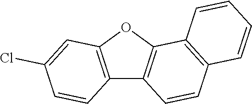 | 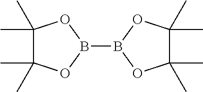 | 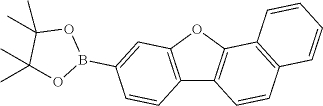 [SMILES: CC1(C)OB(c2ccc3c(c2)oc2c4ccccc4ccc32)OC1(C)C] | 73% | 345.22 |
| B3-14 | 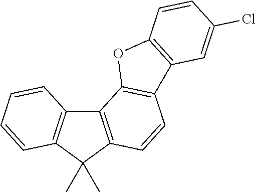 | 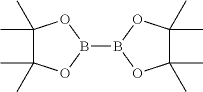 | 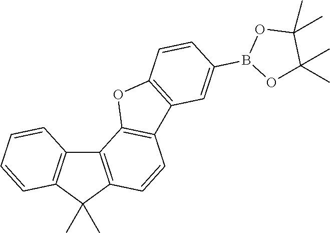 [SMILES: CC1(C)c2ccccc2-c2c1ccc1c2oc2ccc(B3OC(C)(C)C(C)(C)O3)cc21] | 75% | 411.32 |
| B3-15 | B2-1 | 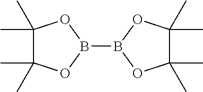 | 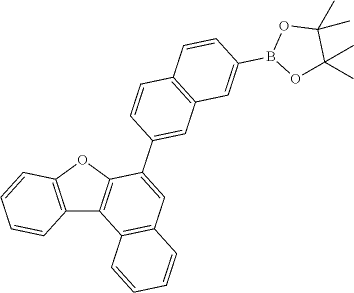 [SMILES: CC1(C)OB(c2ccc3ccc(-c4cc5ccccc5c5c4oc4ccccc45)cc3c2)OC1(C)C] | 77% | 471.38 |
| B3-16 | 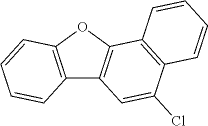 | 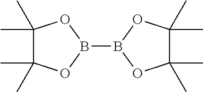 | 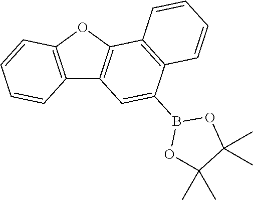 [SMILES: CC1(C)OB(c2cc3c4ccccc4oc3c3ccccc23)OC1(C)C] | 73% | 345.22 |
| B3-17 | 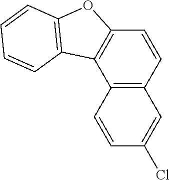 | 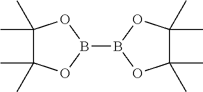 | 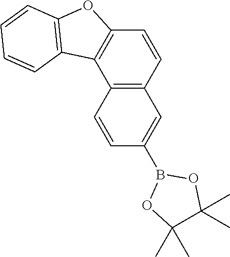 [SMILES: CC1(C)OB(c2ccc3c(ccc4oc5ccccc5c43)c2)OC1(C)C] | 75% | 345.22 |
| B3-18 | 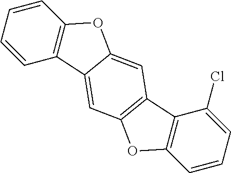 | 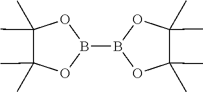 | 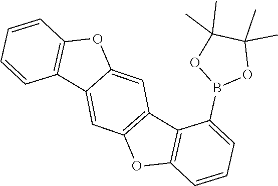 [SMILES: CC1(C)OB(c2cccc3oc4cc5c(cc4c23)oc2ccccc25)OC1(C)C] | 74% | 385.24 |
| B3-19 | 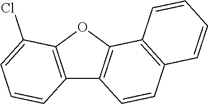 | 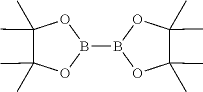 | 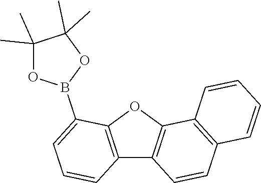 [SMILES: CC1(C)OB(c2cccc3c2oc2c4ccccc4ccc32)OC1(C)C] | 74% | 345.22 |
| B3-20 | B2-2 | 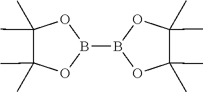 | 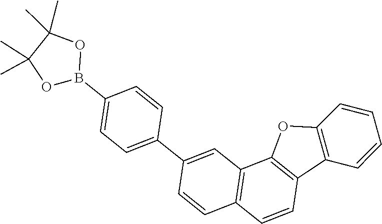 [SMILES: CC1(C)OB(c2ccc(-c3ccc4ccc5c6ccccc6oc5c4c3)cc2)OC1(C)C] | 73% | 421.32 |
| B3-21 | 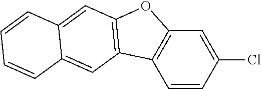 | 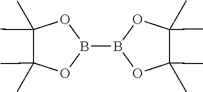 | 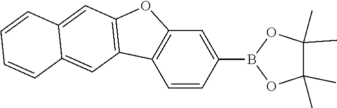 [SMILES: CC1(C)OB(c2ccc3c(c2)oc2cc4ccccc4cc23)OC1(C)C] | 76% | 345.22 |
| B3-22 | 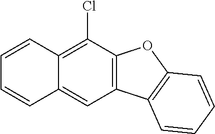 | 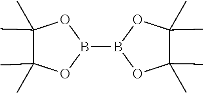 | 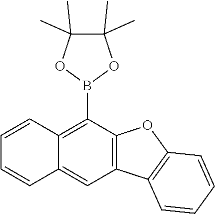 [SMILES: CC1(C)OB(c2c3ccccc3cc3c2oc2ccccc23)OC1(C)C] | 77% | 345.22 |
| B3-23 | 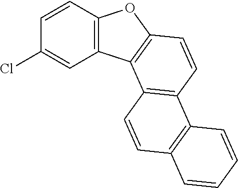 | 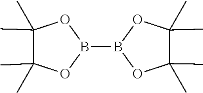 | 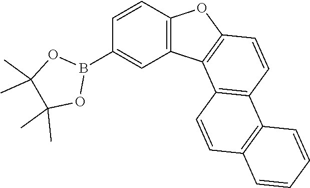 [SMILES: CC1(C)OB(c2ccc3oc4ccc5c6ccccc6ccc5c4c3c2)OC1(C)C] | 73% | 395.28 |
| B3-24 | 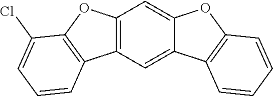 | 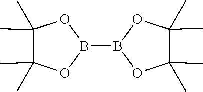 | 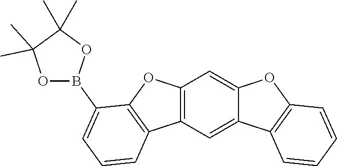 [SMILES: CC1(C)OB(c2cccc3c2oc2cc4oc5ccccc5c4cc23)OC1(C)C] | 70% | 385.24 |
| B3-25 | 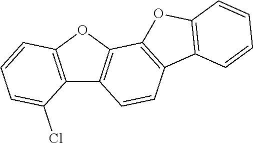 | 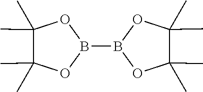 | 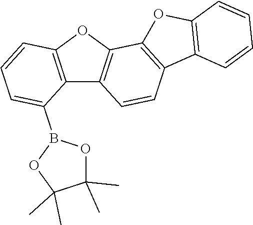 [SMILES: CC1(C)OB(c2cccc3oc4c(ccc5c6ccccc6oc54)c23)OC1(C)C] | 73% | 385.24 |
| B3-26 | 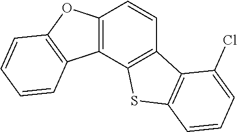 | 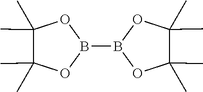 | 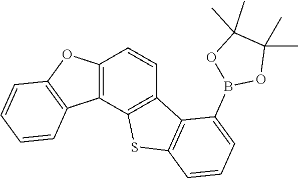 [SMILES: CC1(C)OB(c2cccc3sc4c(ccc5oc6ccccc6c54)c23)OC1(C)C] | 71% | 401.30 |
| B3-27 | 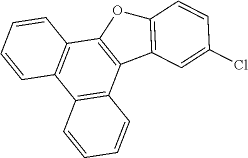 | 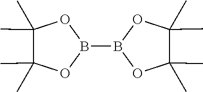 | 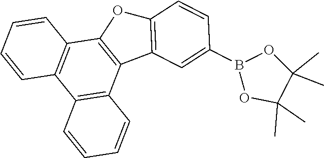 [SMILES: CC1(C)OB(c2ccc3oc4c5ccccc5c5ccccc5c4c3c2)OC1(C)C] | 73% | 395.28 |

### 4) Synthesis of Int 1.

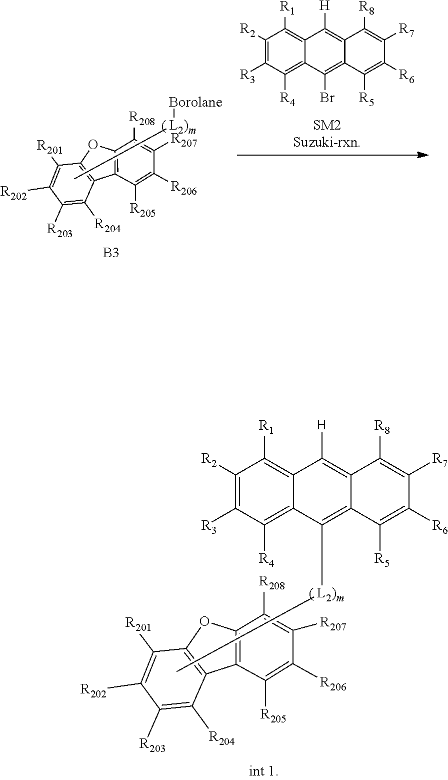

After SM1 (one of B3, 1 eq) and SM2 (1.1 eq or 2.1 eq) were added to tetrahydrofuran (THF) (excess), a 2 M aqueous potassium carbonate solution (30 volume ratio compared to THF) was added thereto, and tetrakis triphenyl-phosphino palladium (2 mol %) was added thereto, and then the resulting mixture was heated and stirred at 85° C. for 10 hours. After the temperature was lowered to room temperature and the reaction was terminated, layers were separated by removing the aqueous potassium carbonate solution, and the residue was recrystallized with chloroform and ethyl acetate to prepare Chemical Formula int. (int 1-1. to int 1-27.) above.

int 1-1 to int 1-27 in [Table 7]were synthesized in the same manner as in the method of synthesizing Chemical Formula int 1., except that SM1 and SM2 were changed to the materials in the following Table 7.

TABLE 7

| | | | | | MS |
|---|---|---|---|---|---|
| | SM1 | SM2 | Product | Yield | [M + H]+ |
| int 1-1 | 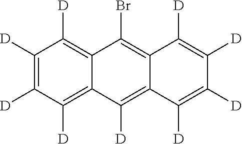 | B3-1 | 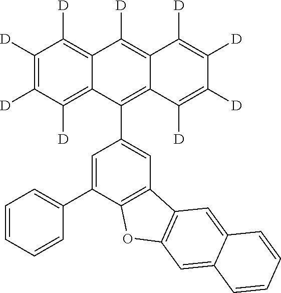 [SMILES: [2H]c1c([2H])c([2H])c2c(-c3cc(-c4ccccc4)c4oc5cc6ccccc6cc5c4c3)c3c([2H])c([2H])c([2H])c([2H])c3c([2H])c2c1[2H]] | 75% | 480.63 |
| int 1-2 | 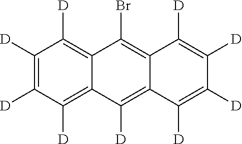 | B3-2 | 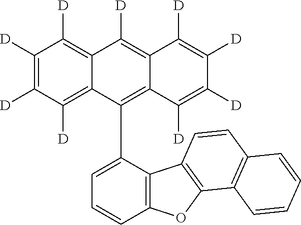 [SMILES: [2H]c1c([2H])c([2H])c2c(-c3cccc4oc5c6ccccc6ccc5c34)c3c([2H])c([2H])c([2H])c([2H])c3c([2H])c2c1[2H]] | 76% | 404.53 |
| int 1-3 | 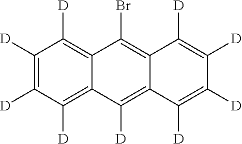 | B3-3 | 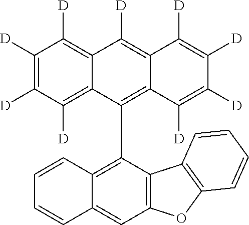 [SMILES: [2H]c1c([2H])c([2H])c2c(-c3c4ccccc4cc4oc5ccccc5c34)c3c([2H])c([2H])c([2H])c([2H])c3c([2H])c2c1[2H]] | 73% | 404.53 |
| int 1-4 | 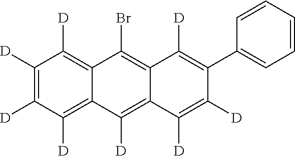 | B3-4 | 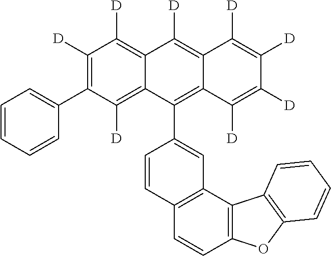 [SMILES: [2H]c1c([2H])c([2H])c2c(-c3ccc4ccc5oc6ccccc6c5c4c3)c3c([2H])c(-c4ccccc4)c([2H])c([2H])c3c([2H])c2c1[2H]] | 70% | 479.62 |
| int 1-5 | 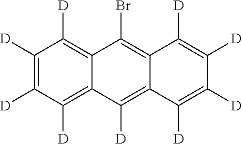 | B3-5 | 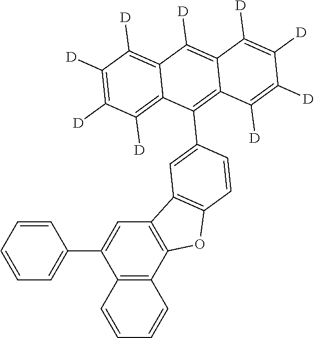 [SMILES: [2H]c1c([2H])c([2H])c2c(-c3ccc4oc5c6ccccc6c(-c6ccccc6)cc5c4c3)c3c([2H])c([2H])c([2H])c([2H])c3c([2H])c2c1[2H]] | 71% | 480.63 |
| int 1-6 | 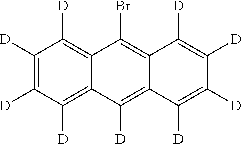 | B3-6 | 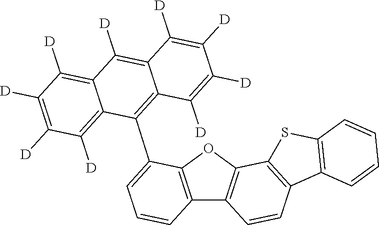 [SMILES: [2H]c1c([2H])c([2H])c2c(-c3cccc4c3oc3c4ccc4c5ccccc5sc43)c3c([2H])c([2H])c([2H])c([2H])c3c([2H])c2c1[2H]] | 73% | 460.61 |
| int 1-7 | 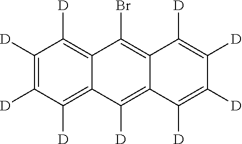 | B3-7 | 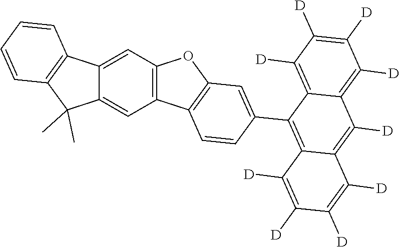 [SMILES: [2H]c1c([2H])c([2H])c2c(-c3ccc4c(c3)oc3cc5c(cc34)C(C)(C)c3ccccc3-5)c3c([2H])c([2H])c([2H])c([2H])c3c([2H])c2c1[2H]] | 75% | 470.63 |
| int 1-8 | 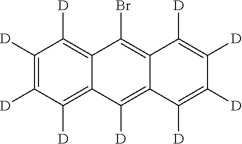 | B3-8 | 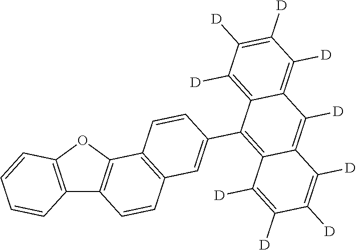 [SMILES: [2H]c1c([2H])c([2H])c2c(-c3ccc4c(ccc5c6ccccc6oc45)c3)c3c([2H])c([2H])c([2H])c([2H])c3c([2H])c2c1[2H]] | 71% | 404.53 |
| int 1-9 | 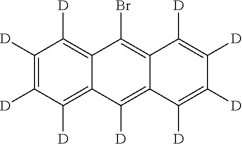 | B3-9 | 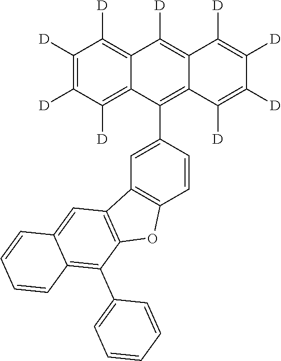 [SMILES: [2H]c1c([2H])c([2H])c2c(-c3ccc4oc5c(-c6ccccc6)c6ccccc6cc5c4c3)c3c([2H])c([2H])c([2H])c([2H])c3c([2H])c2c1[2H]] | 73% | 480.63 |
| int 1-10 | 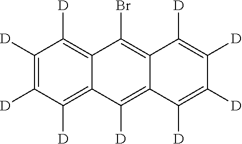 | B3-10 | 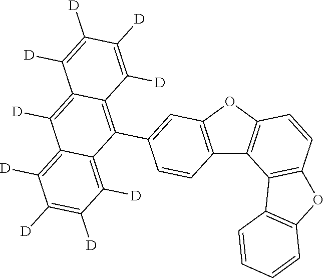 [SMILES: [2H]c1c([2H])c([2H])c2c(-c3ccc4c(c3)oc3ccc5oc6ccccc6c5c34)c3c([2H])c([2H])c([2H])c([2H])c3c([2H])c2c1[2H]] | 74% | 444.55 |
| int 1-11 | 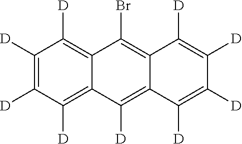 | B3-11 | 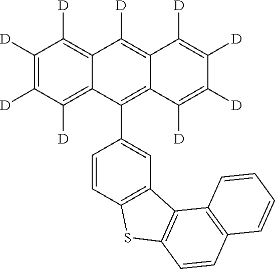 [SMILES: [2H]c1c([2H])c([2H])c2c(-c3ccc4sc5ccc6ccccc6c5c4c3)c3c([2H])c([2H])c([2H])c([2H])c3c([2H])c2c1[2H]] | 70% | 420.59 |
| int 1-12 | 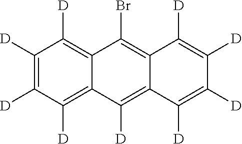 | B3-12 | 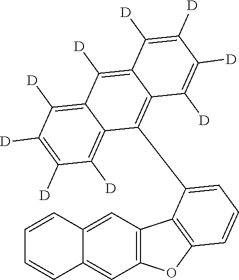 [SMILES: [2H]c1c([2H])c([2H])c2c(-c3cccc4oc5cc6ccccc6cc5c34)c3c([2H])c([2H])c([2H])c([2H])c3c([2H])c2c1[2H]] | 73% | 404.53 |
| int 1-13 | 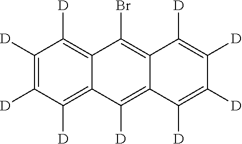 | B3-13 | 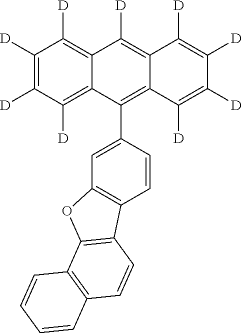 [SMILES: [2H]c1c([2H])c([2H])c2c(-c3ccc4c(c3)oc3c5ccccc5ccc43)c3c([2H])c([2H])c([2H])c([2H])c3c([2H])c2c1[2H]] | 75% | 404.53 |
| int 1-14 | 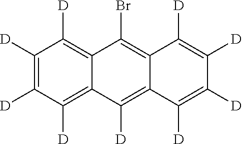 | B3-14 | 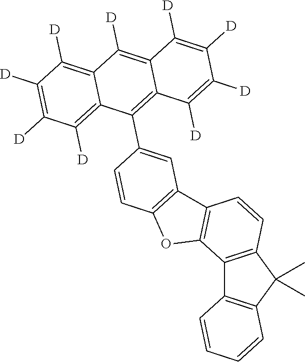 [SMILES: [2H]c1c([2H])c([2H])c2c(-c3ccc4oc5c6c(ccc5c4c3)C(C)(C)c3ccccc3-6)c3c([2H])c([2H])c([2H])c([2H])c3c([2H])c2c1[2H]] | 71% | 470.63 |
| int 1-15 | 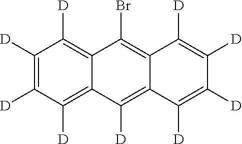 | B3-15 | 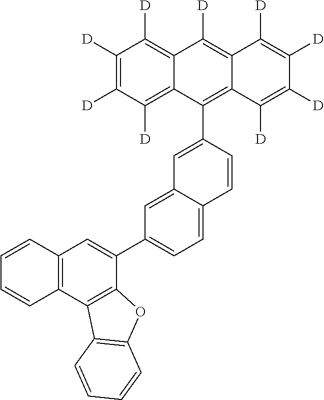 [SMILES: [2H]c1c([2H])c([2H])c2c(-c3ccc4ccc(-c5cc6ccccc6c6c5oc5ccccc56)cc4c3)c3c([2H])c([2H])c([2H])c([2H])c3c([2H])c2c1[2H]] | 75% | 530.69 |
| int 1-16 | 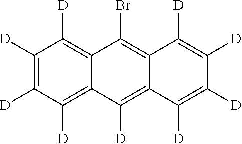 | B3-16 | 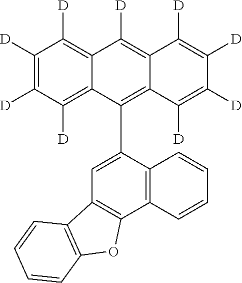 [SMILES: [2H]c1c([2H])c([2H])c2c(-c3cc4c5ccccc5oc4c4ccccc34)c3c([2H])c([2H])c([2H])c([2H])c3c([2H])c2c1[2H]] | 70% | 404.53 |
| int 1-17 | 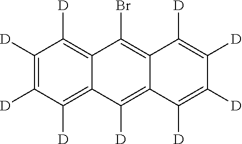 | B3-17 | 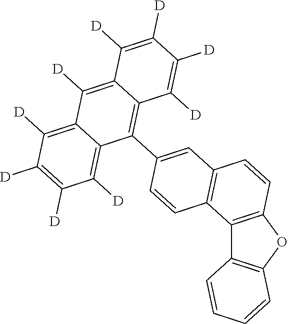 [SMILES: [2H]c1c([2H])c([2H])c2c(-c3ccc4c(ccc5oc6ccccc6c54)c3)c3c([2H])c([2H])c([2H])c([2H])c3c([2H])c2c1[2H]] | 73% | 404.53 |
| int 1-18 | 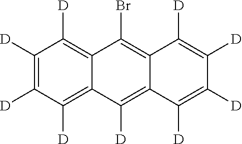 | B3-18 | 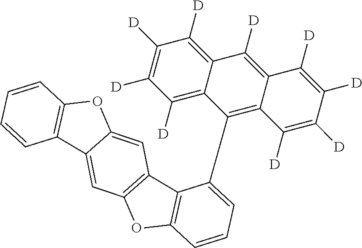 [SMILES: [2H]c1c([2H])c([2H])c2c(-c3cccc4oc5cc6c(cc5c34)oc3ccccc36)c3c([2H])c([2H])c([2H])c([2H])c3c([2H])c2c1[2H]] | 71% | 460.59 |
| int 1-19 | 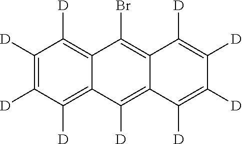 | B3-19 | 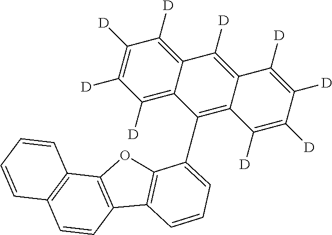 [SMILES: [2H]c1c([2H])c([2H])c2c(-c3cccc4c3oc3c5ccccc5ccc43)c3c([2H])c([2H])c([2H])c([2H])c3c([2H])c2c1[2H]] | 71% | 404.53 |
| int 1-20 | 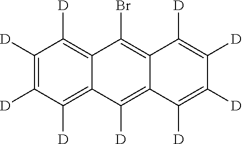 | B3-20 | 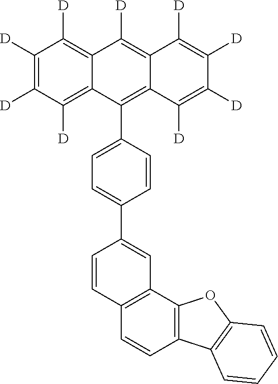 [SMILES: [2H]c1c([2H])c([2H])c2c(-c3ccc(-c4ccc5ccc6c7ccccc7oc6c5c4)cc3)c3c([2H])c([2H])c([2H])c([2H])c3c([2H])c2c1[2H]] | 70% | 480.63 |
| int 1-21 | 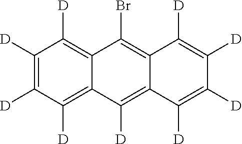 | B3-21 | 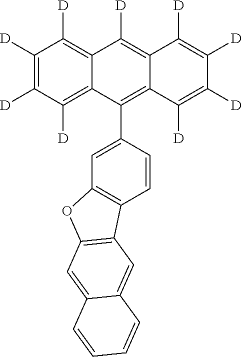 [SMILES: [2H]c1c([2H])c([2H])c2c(-c3ccc4c(c3)oc3cc5ccccc5cc34)c3c([2H])c([2H])c([2H])c([2H])c3c([2H])c2c1[2H]] | 73% | 404.53 |
| int 1-22 | 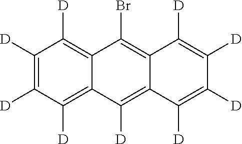 | B3-22 | 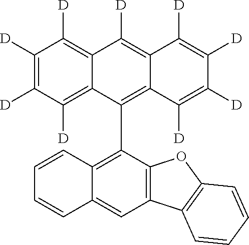 [SMILES: [2H]c1c([2H])c([2H])c2c(-c3c4ccccc4cc4c3oc3ccccc34)c3c([2H])c([2H])c([2H])c([2H])c3c([2H])c2c1[2H]] | 75% | 404.53 |
| int 1-23 | 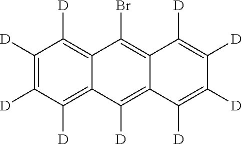 | B3-23 | 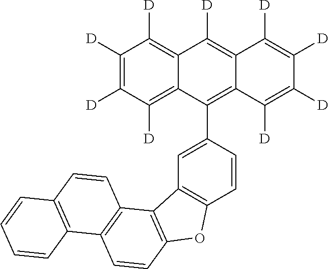 [SMILES: [2H]c1c([2H])c([2H])c2c(-c3ccc4oc5ccc6c7ccccc7ccc6c5c4c3)c3c([2H])c([2H])c([2H])c([2H])c3c([2H])c2c1[2H]] | 76% | 454.59 |
| int 1-24 | 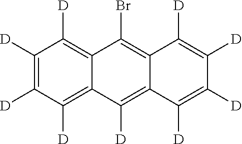 | B3-24 | 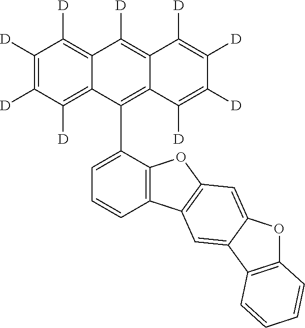 [SMILES: [2H]c1c([2H])c([2H])c2c(-c3cccc4c3oc3cc5oc6ccccc6c5cc34)c3c([2H])c([2H])c([2H])c([2H])c3c([2H])c2c1[2H]] | 77% | 444.55 |
| int 1-25 | 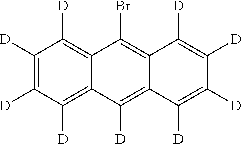 | B3-25 | 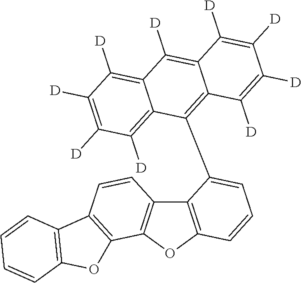 [SMILES: [2H]c1c([2H])c([2H])c2c(-c3cccc4oc5c(ccc6c7ccccc7oc65)c34)c3c([2H])c([2H])c([2H])c([2H])c3c([2H])c2c1[2H]] | 72% | 444.55 |
| int 1-26 | 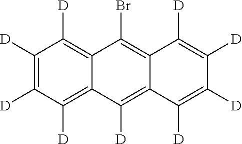 | B3-26 | 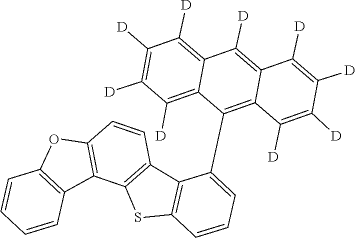 [SMILES: [2H]c1c([2H])c([2H])c2c(-c3cccc4sc5c(ccc6oc7ccccc7c65)c34)c3c([2H])c([2H])c([2H])c([2H])c3c([2H])c2c1[2H]] | 73% | 460.61 |
| int 1-27 | 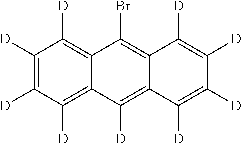 | B3-27 | 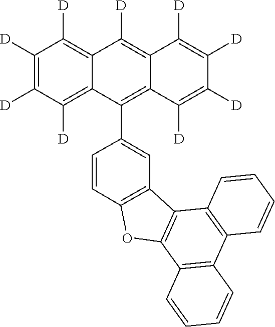 [SMILES: [2H]c1c([2H])c([2H])c2c(-c3ccc4oc5c6ccccc6c6ccccc6c5c4c3)c3c([2H])c([2H])c([2H])c([2H])c3c([2H])c2c1[2H]] | 75% | 454.59 |

### 5) Synthesis of Int 2.

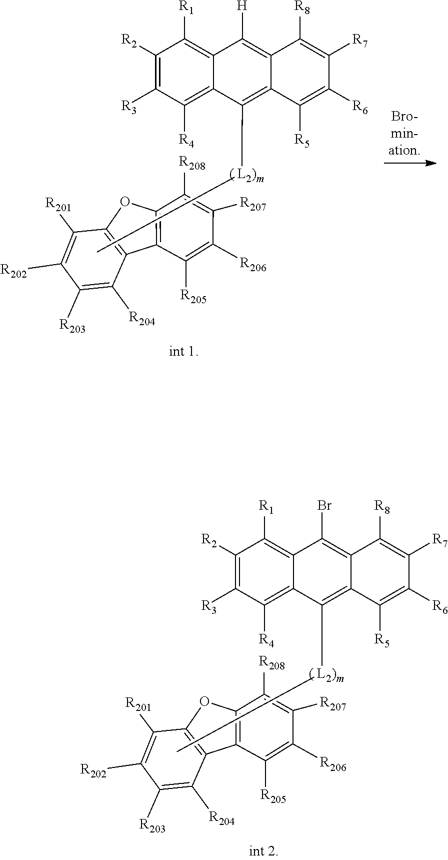

After SM1 (one of int 1., 1 eq) was dissolved in chloroform (excess), the temperature was lowered to 0° C. and stabilized, and then N-bromosuccinimide (1 eq) was dissolved in DMF (excess), and the resulting solution was added dropwise thereto. Thereafter, the reactant was warmed to room temperature and stirred for 1 hour, and 1 N HCl (excess) was added thereto to terminate the reaction. After the reaction was completed, the solvent was removed by separating layers, and then the residue was subjected to silica column chromatography (ethyl acetate/hexane 1:15) to prepare Chemical Formula int 2. (int 2-1. to int 2-27.).

int 2-1. to t 2-27. in Table 8 were synthesized in the same manner as in the method of synthesizing Chemical Formula it 2., except that SM and SM2 were changed to the materials in the following Table 8.

TABLE 8

| | | | | | MS |
|---|---|---|---|---|---|
| | SM1 | SM2 | Product | Yield | [M + H]+ |
| int 2-1 | int 1-1 | NBS (1 eq) chloroform, DMF | 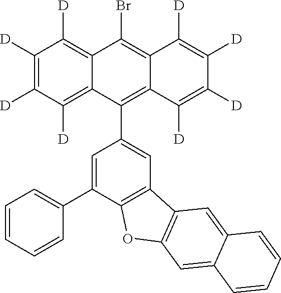 [SMILES: [2H]c1c([2H])c([2H])c2c(-c3cc(-c4ccccc4)c4oc5cc6ccccc6cc5c4c3)c3c([2H])c([2H])c([2H])c([2H])c3c(Br)c2c1[2H]] | 85% | 558.52 |
| int 2-2 | int 1-2 | NBS (1 eq) chloroform, DMF | 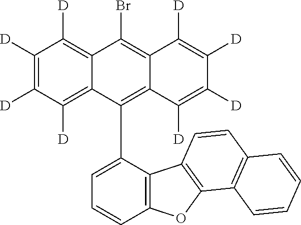 [SMILES: [2H]c1c([2H])c([2H])c2c(-c3cccc4oc5c6ccccc6ccc5c34)c3c([2H])c([2H])c([2H])c([2H])c3c(Br)c2c1[2H]] | 80% | 482.42 |
| int 2-3 | int 1-3 | NBS (1 eq) chloroform, DMF | 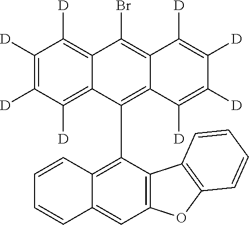 [SMILES: [2H]c1c([2H])c([2H])c2c(-c3c4ccccc4cc4oc5ccccc5c34)c3c([2H])c([2H])c([2H])c([2H])c3c(Br)c2c1[2H]] | 81% | 482.42 |
| int 2-4 | int 1-4 | NBS (1 eq) chloroform, DMF | 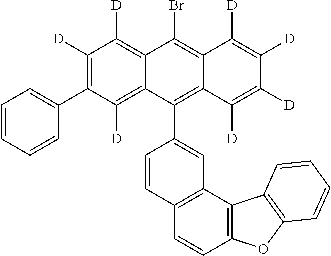 [SMILES: [2H]c1c([2H])c([2H])c2c(-c3ccc4ccc5oc6ccccc6c5c4c3)c3c([2H])c(-c4ccccc4)c([2H])c([2H])c3c(Br)c2c1[2H]] | 83% | 557.51 |
| int 2-5 | int 1-5 | NBS (1 eq) chloroform, DMF | 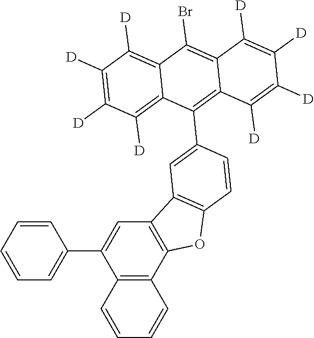 [SMILES: [2H]c1c([2H])c([2H])c2c(-c3ccc4oc5c6ccccc6c(-c6ccccc6)cc5c4c3)c3c([2H])c([2H])c([2H])c([2H])c3c(Br)c2c1[2H]] | 80% | 558.52 |
| int 2-6 | int 1-6 | NBS (1 eq) chloroform, DMF | 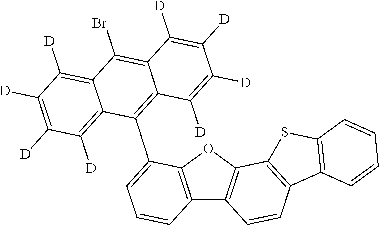 [SMILES: [2H]c1c([2H])c([2H])c2c(-c3cccc4c3oc3c4ccc4c5ccccc5sc43)c3c([2H])c([2H])c([2H])c([2H])c3c(Br)c2c1[2H]] | 84% | 538.50 |
| int 2-7 | int 1-7 | NBS (1 eq) chloroform, DMF | 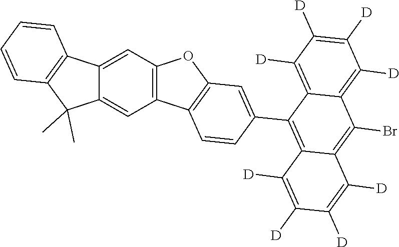 [SMILES: [2H]c1c([2H])c([2H])c2c(-c3ccc4c(c3)oc3cc5c(cc34)C(C)(C)c3ccccc3-5)c3c([2H])c([2H])c([2H])c([2H])c3c(Br)c2c1[2H]] | 80% | 548.52 |
| int 2-8 | int 1-8 | NBS (1 eq) chloroform, DMF | 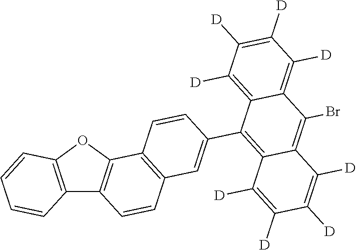 [SMILES: [2H]c1c([2H])c([2H])c2c(-c3ccc4c(ccc5c6ccccc6oc45)c3)c3c([2H])c([2H])c([2H])c([2H])c3c(Br)c2c1[2H]] | 79% | 482.42 |
| int 2-9 | int 1-9 | NBS (leq) chloroform, DMF | 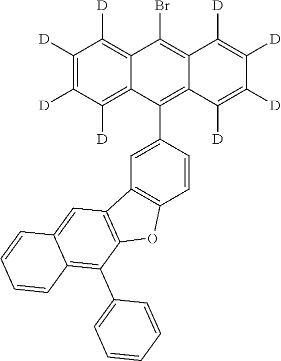 [SMILES: [2H]c1c([2H])c([2H])c2c(-c3ccc4oc5c(-c6ccccc6)c6ccccc6cc5c4c3)c3c([2H])c([2H])c([2H])c([2H])c3c(Br)c2c1[2H]] | 80% | 558.52 |
| int 2-10 | int 1-10 | NBS (leq) chloroform, DMF | 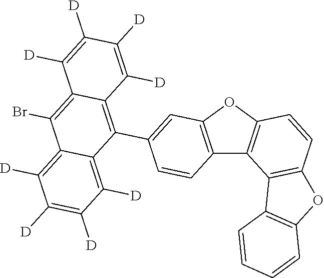 [SMILES: [2H]c1c([2H])c([2H])c2c(-c3ccc4c(c3)oc3ccc5oc6ccccc6c5c34)c3c([2H])c([2H])c([2H])c([2H])c3c(Br)c2c1[2H]] | 79% | 522.44 |
| int 2-11 | int 1-11 | NBS (leq) chloroform, DMF | 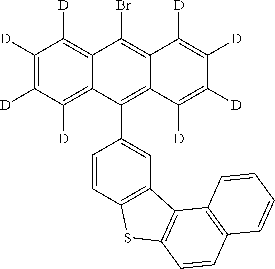 [SMILES: [2H]c1c([2H])c([2H])c2c(-c3ccc4sc5ccc6ccccc6c5c4c3)c3c([2H])c([2H])c([2H])c([2H])c3c(Br)c2c1[2H]] | 83% | 498.48 |
| int 2-12 | int 1-12 | NBS (1 eq) chloroform, DMF | 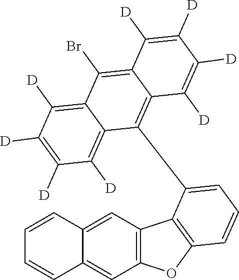 [SMILES: [2H]c1c([2H])c([2H])c2c(-c3cccc4oc5cc6ccccc6cc5c34)c3c([2H])c([2H])c([2H])c([2H])c3c(Br)c2c1[2H]] | 80% | 482.42 |
| int 2-13 | int 1-13 | NBS (1 eq) chloroform, DMF | 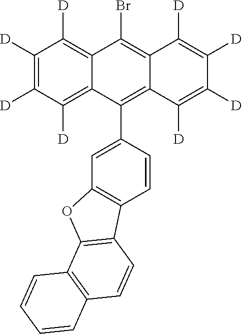 [SMILES: [2H]c1c([2H])c([2H])c2c(-c3ccc4c(c3)oc3c5ccccc5ccc43)c3c([2H])c([2H])c([2H])c([2H])c3c(Br)c2c1[2H]] | 81% | 482.42 |
| int 2-14 | int 1-14 | NBS (1 eq) chloroform, DMF | 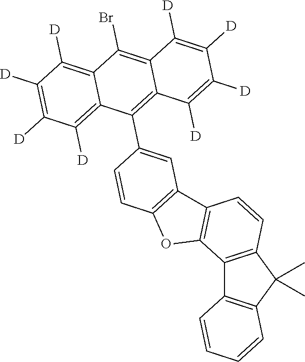 [SMILES: [2H]c1c([2H])c([2H])c2c(-c3ccc4oc5c6c(ccc5c4c3)C(C)(C)c3ccccc3-6)c3c([2H])c([2H])c([2H])c([2H])c3c(Br)c2c1[2H]] | 82% | 548.52 |
| int 2-15 | int 1-15 | NBS (1 eq) chloroform, DMF | 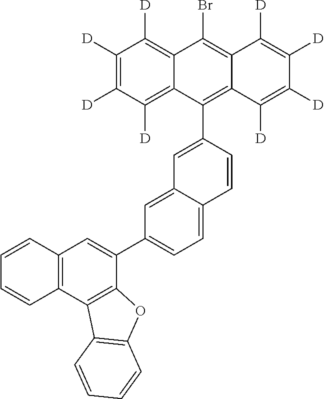 [SMILES: [2H]c1c([2H])c([2H])c2c(-c3ccc4ccc(-c5cc6ccccc6c6c5oc5ccccc56)cc4c3)c3c([2H])c([2H])c([2H])c([2H])c3c(Br)c2c1[2H]] | 83% | 608.58 |
| int 2-16 | int 1-16 | NBS (1 eq) chloroform, DMF | 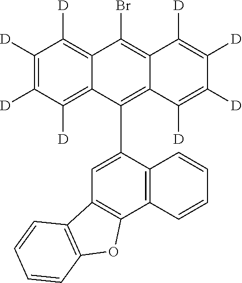 [SMILES: [2H]c1c([2H])c([2H])c2c(-c3cc4c5ccccc5oc4c4ccccc34)c3c([2H])c([2H])c([2H])c([2H])c3c(Br)c2c1[2H]] | 84% | 482.42 |
| int 2-17 | int 1-17 | NBS (1 eq) chloroform, DMF | 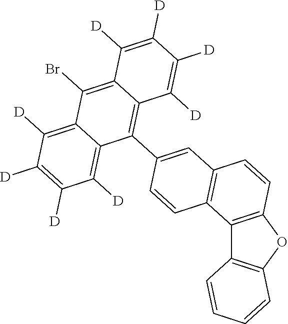 [SMILES: [2H]c1c([2H])c([2H])c2c(-c3ccc4c(ccc5oc6ccccc6c54)c3)c3c([2H])c([2H])c([2H])c([2H])c3c(Br)c2c1[2H]] | 85% | 482.42 |
| int 2-18 | int 1-18 | NBS (1 eq) chloroform, DMF | 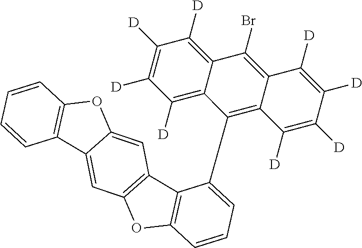 [SMILES: [2H]c1c([2H])c([2H])c2c(-c3cccc4oc5cc6c(cc5c34)oc3ccccc36)c3c([2H])c([2H])c([2H])c([2H])c3c(Br)c2c1[2H]] | 80% | 538.48 |
| int 2-19 | int 1-19 | NBS (leq) chloroform, DMF | 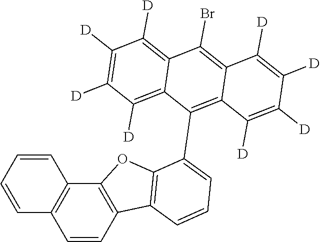 [SMILES: [2H]c1c([2H])c([2H])c2c(-c3cccc4c3oc3c5ccccc5ccc43)c3c([2H])c([2H])c([2H])c([2H])c3c(Br)c2c1[2H]] | 83% | 482.42 |
| int 2-20 | int 1-20 | NBS (leq) chloroform, DMF | 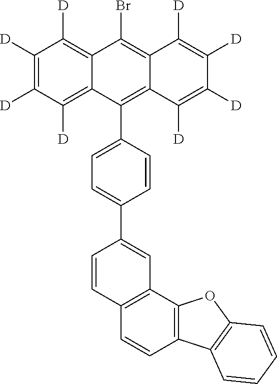 [SMILES: [2H]c1c([2H])c([2H])c2c(-c3ccc(-c4ccc5ccc6c7ccccc7oc6c5c4)cc3)c3c([2H])c([2H])c([2H])c([2H])c3c(Br)c2c1[2H]] | 80% | 558.52 |
| int 2-21 | int 1-21 | NBS (1 eq) chloroform, DMF | 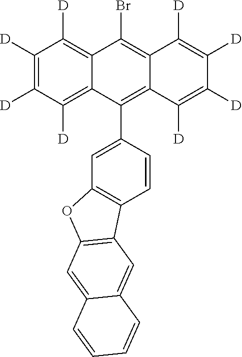 [SMILES: [2H]c1c([2H])c([2H])c2c(-c3ccc4c(c3)oc3cc5ccccc5cc34)c3c([2H])c([2H])c([2H])c([2H])c3c(Br)c2c1[2H]] | 84% | 482.42 |
| int 2-22 | int 1-22 | NBS (1 eq) chloroform, DMF | 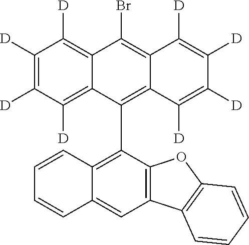 [SMILES: [2H]c1c([2H])c([2H])c2c(-c3c4ccccc4cc4c3oc3ccccc34)c3c([2H])c([2H])c([2H])c([2H])c3c(Br)c2c1[2H]] | 83% | 482.42 |
| int 2-23 | int 1-23 | NBS (1 eq) chloroform, DMF | 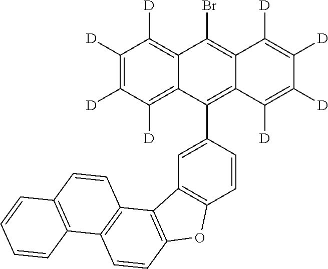 [SMILES: [2H]c1c([2H])c([2H])c2c(-c3ccc4oc5ccc6c7ccccc7ccc6c5c4c3)c3c([2H])c([2H])c([2H])c([2H])c3c(Br)c2c1[2H]] | 81% | 532.48 |
| int 2-24 | int 1-24 | NBS (1 eq) chloroform, DMF | 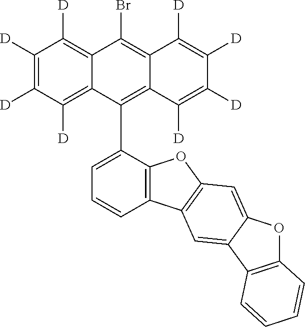 [SMILES: [2H]c1c([2H])c([2H])c2c(-c3cccc4c3oc3cc5oc6ccccc6c5cc34)c3c([2H])c([2H])c([2H])c([2H])c3c(Br)c2c1[2H]] | 80% | 522.44 |
| int 2-25 | int 1-25 | NBS (1 eq) chloroform, DMF | 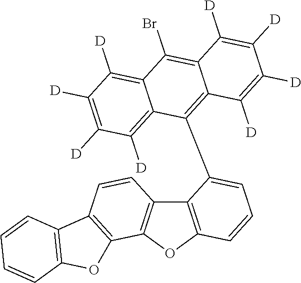 [SMILES: [2H]c1c([2H])c([2H])c2c(-c3cccc4oc5c(ccc6c7ccccc7oc65)c34)c3c([2H])c([2H])c([2H])c([2H])c3c(Br)c2c1[2H]] | 84% | 522.44 |
| int 2-26 | int 1-26 | NBS (1 eq) chloroform, DMF | 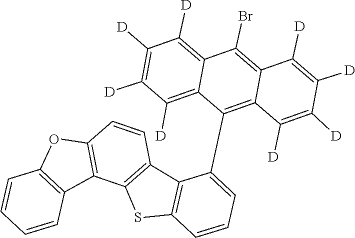 [SMILES: [2H]c1c([2H])c([2H])c2c(-c3cccc4sc5c(ccc6oc7ccccc7c65)c34)c3c([2H])c([2H])c([2H])c([2H])c3c(Br)c2c1[2H]] | 80% | 538.50 |
| int 2-27 | int 1-27 | NBS (1 eq) chloroform, DMF | 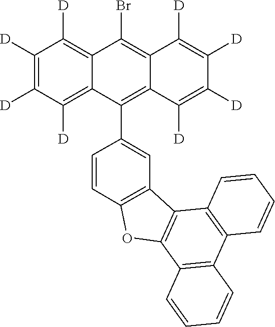 [SMILES: [2H]c1c([2H])c([2H])c2c(-c3ccc4oc5c6ccccc6c6ccccc6c5c4c3)c3c([2H])c([2H])c([2H])c([2H])c3c(Br)c2c1[2H]] | 83% | 532.48 |

### 6) Synthesis of Chemical Formula 1

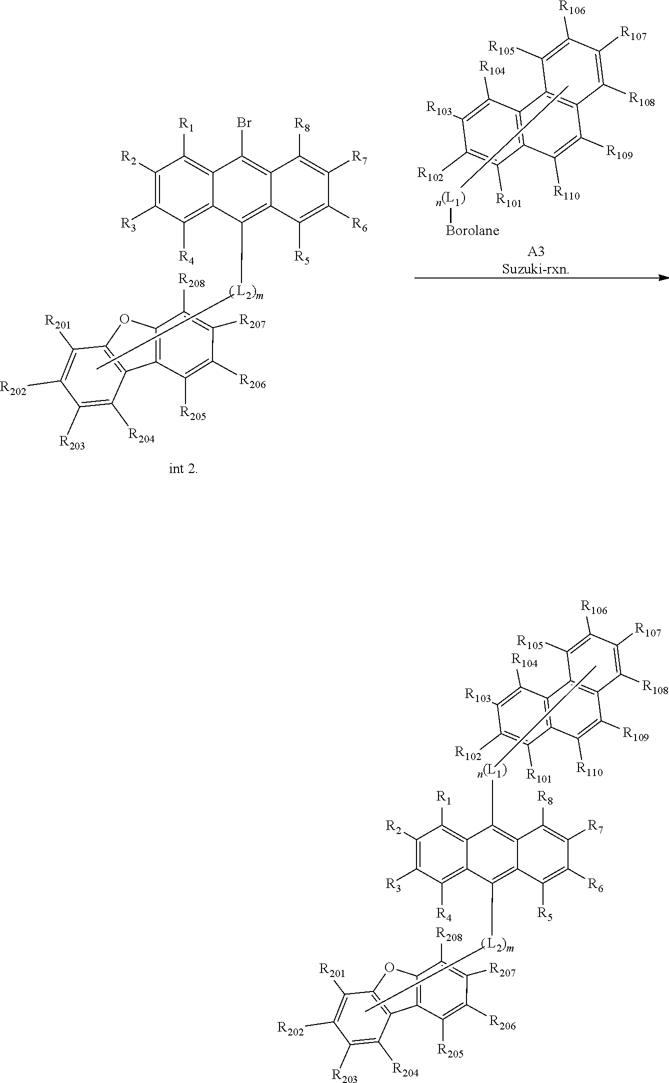

After SM1 (one of int 2., 1 eq) and SM2 (1.1 eq) were added to tetrahydrofuran (THF) (excess), a 2 M aqueous potassium carbonate solution (30 volume ratio compared to THF) was added thereto, and tetrakis triphenyl-phosphino palladium (2 mol %) was added thereto, and then the resulting mixture was heated and stirred at 85° C. for 10 hours. After the temperature was lowered to room temperature and the reaction was terminated, layers were separated by removing the aqueous potassium carbonate solution, and the residue was columned with hexane and ethyl acetate to prepare Chemical Formula 1 (Compounds 1 to 27) above.

Compounds 1 to 27 in [Table 9]were synthesized in the same manner as in the method of synthesizing Chemical Formula int., except that SM1 and SM2 were changed to the materials in the following Table 9.

TABLE 9

| | | | | | MS |
|---|---|---|---|---|---|
| Compound | SM1 | SM2 | Product | Yield | [M + H]+ |
| 1 | int 2-1 | 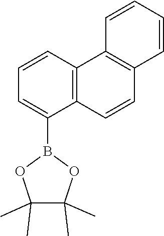 | 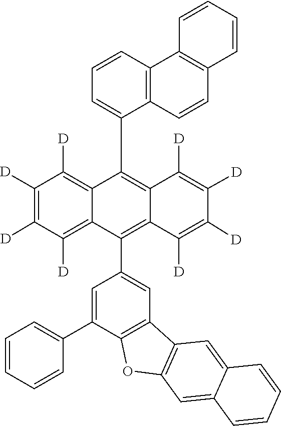 [SMILES: [2H]c1c([2H])c([2H])c2c(-c3cccc4c3ccc3ccccc34)c3c([2H])c([2H])c([2H])c([2H])c3c(-c3cc(-c4ccccc4)c4oc5cc6ccccc6cc5c4c3)c2c1[2H]] | 67% | 655.84 |
| 2 | int 2-2 | 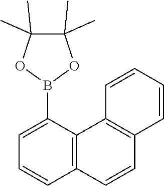 | 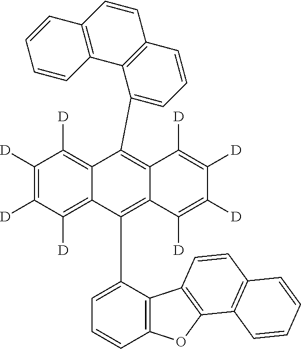 [SMILES: [2H]c1c([2H])c([2H])c2c(-c3cccc4oc5c6ccccc6ccc5c34)c3c([2H])c([2H])c([2H])c([2H])c3c(-c3cccc4ccc5ccccc5c34)c2c1[2H]] | 65% | 579.74 |
| 3 | int 2-3 | 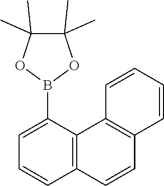 | 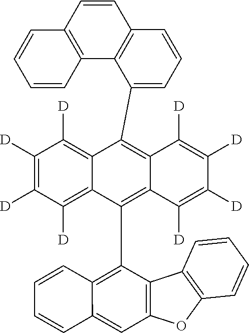 [SMILES: [2H]c1c([2H])c([2H])c2c(-c3c4ccccc4cc4oc5ccccc5c34)c3c([2H])c([2H])c([2H])c([2H])c3c(-c3cccc4ccc5ccccc5c34)c2c1[2H]] | 68% | 579.74 |
| 4 | int 2-4 | 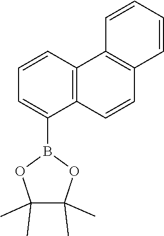 | 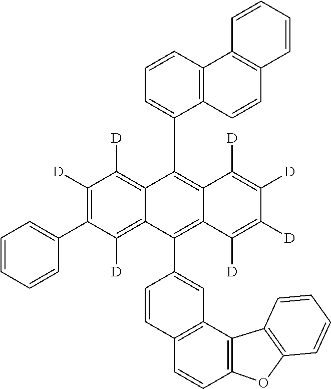 [SMILES: [2H]c1c([2H])c([2H])c2c(-c3cccc4c3ccc3ccccc34)c3c([2H])c([2H])c(-c4ccccc4)c([2H])c3c(-c3ccc4ccc5oc6ccccc6c5c4c3)c2c1[2H]] | 65% | 654.83 |
| 5 | int 2-5 | 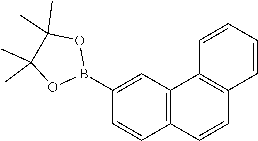 | 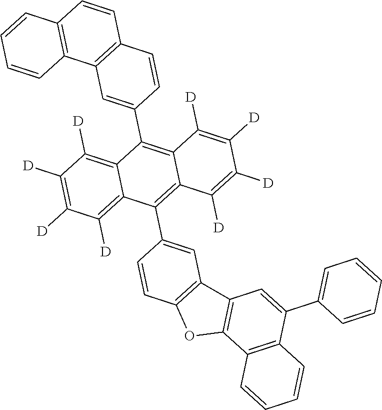 [SMILES: [2H]c1c([2H])c([2H])c2c(-c3ccc4oc5c6ccccc6c(-c6ccccc6)cc5c4c3)c3c([2H])c([2H])c([2H])c([2H])c3c(-c3ccc4ccc5ccccc5c4c3)c2c1[2H]] | 66% | 655.84 |
| 6 | int 2-6 | 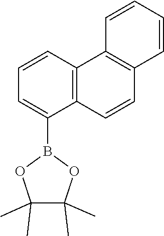 | 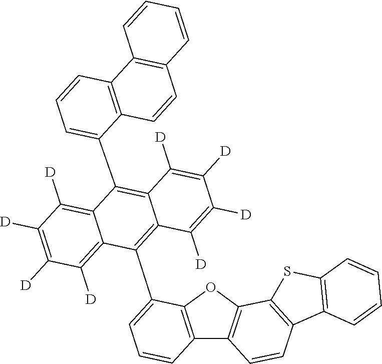 [SMILES: [2H]c1c([2H])c([2H])c2c(-c3cccc4c3oc3c4ccc4c5ccccc5sc43)c3c([2H])c([2H])c([2H])c([2H])c3c(-c3cccc4c3ccc3ccccc34)c2c1[2H]] | 63% | 635.82 |
| 7 | int 2-7 | 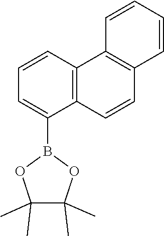 | 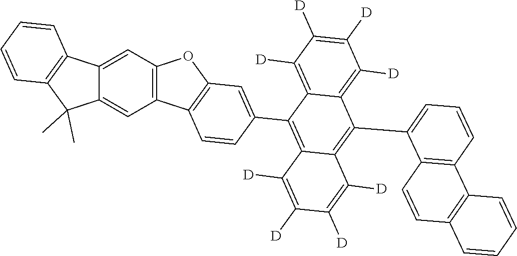 [SMILES: [2H]c1c([2H])c([2H])c2c(-c3cccc4c3ccc3ccccc34)c3c([2H])c([2H])c([2H])c([2H])c3c(-c3ccc4c(c3)oc3cc5c(cc34)C(C)(C)c3ccccc3-5)c2c1[2H]] | 65% | 645.84 |
| 8 | int 2-8 | 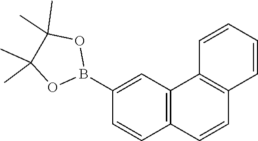 | 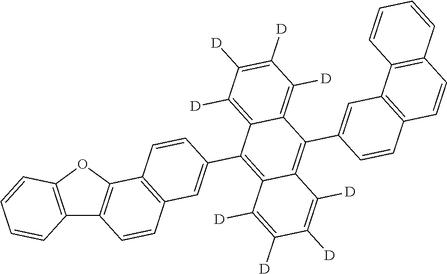 [SMILES: [2H]c1c([2H])c([2H])c2c(-c3ccc4ccc5ccccc5c4c3)c3c([2H])c([2H])c([2H])c([2H])c3c(-c3ccc4c(ccc5c6ccccc6oc45)c3)c2c1[2H]] | 67% | 579.74 |
| 9 | int 2-9 | 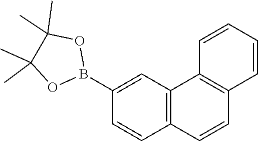 | 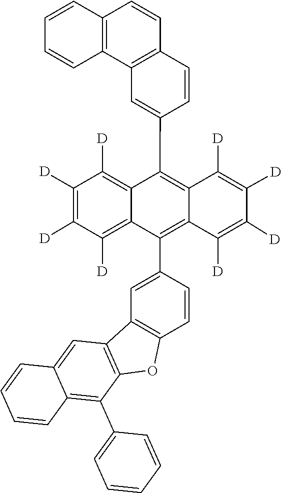 [SMILES: [2H]c1c([2H])c([2H])c2c(-c3ccc4oc5c(-c6ccccc6)c6ccccc6cc5c4c3)c3c([2H])c([2H])c([2H])c([2H])c3c(-c3ccc4ccc5ccccc5c4c3)c2c1[2H]] | 70% | 655.84 |
| 10 | int 2-10 | 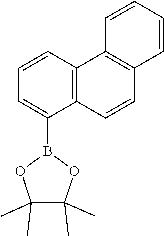 | 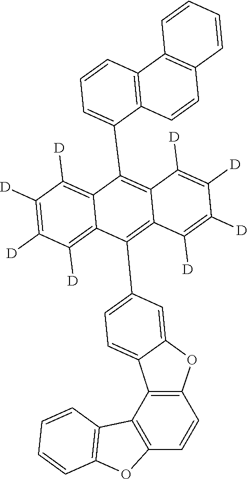 [SMILES: [2H]c1c([2H])c([2H])c2c(-c3cccc4c3ccc3ccccc34)c3c([2H])c([2H])c([2H])c([2H])c3c(-c3ccc4c(c3)oc3ccc5oc6ccccc6c5c34)c2c1[2H]] | 63% | 619.76 |
| 11 | int 2-11 | A3-1 | 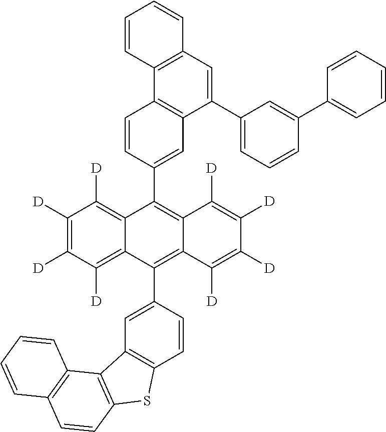 [SMILES: [2H]c1c([2H])c([2H])c2c(-c3ccc4sc5ccc6ccccc6c5c4c3)c3c([2H])c([2H])c([2H])c([2H])c3c(-c3ccc4c(c3)c(-c3cccc(-c5ccccc5)c3)cc3ccccc34)c2c1[2H]] | 65% | 748.00 |
| 12 | int 2-12 | A3-2 | 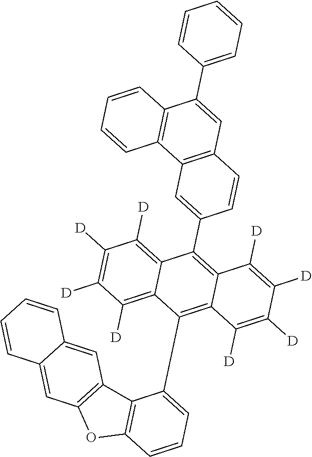 [SMILES: [2H]c1c([2H])c([2H])c2c(-c3cccc4oc5cc6ccccc6cc5c34)c3c([2H])c([2H])c([2H])c([2H])c3c(-c3ccc4cc(-c5ccccc5)c5ccccc5c4c3)c2c1[2H]] | 64% | 655.84 |
| 13 | int 2-13 | A3-3 | 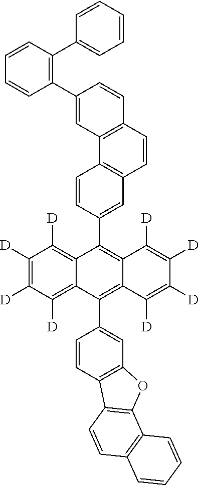 [SMILES: [2H]c1c([2H])c([2H])c2c(-c3ccc4c(c3)oc3c5ccccc5ccc43)c3c([2H])c([2H])c([2H])c([2H])c3c(-c3ccc4c(ccc5ccc(-c6ccccc6-c6ccccc6)cc54)c3)c2c1[2H]] | 60% | 731.94 |
| 14 | int 2-14 | A3-4 | 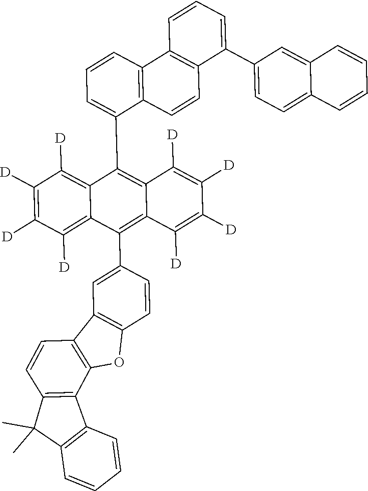 [SMILES: [2H]c1c([2H])c([2H])c2c(-c3cccc4c3ccc3c(-c5ccc6ccccc6c5)cccc34)c3c([2H])c([2H])c([2H])c([2H])c3c(-c3ccc4oc5c6c(ccc5c4c3)C(C)(C)c3ccccc3-6)c2c1[2H]] | 63% | 772.00 |
| 15 | int 2-15 | 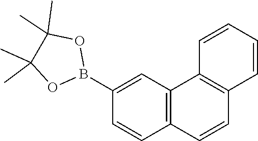 | 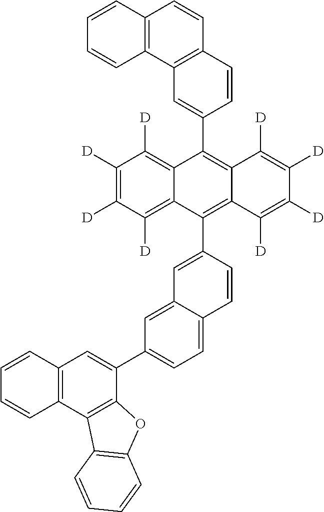 [SMILES: [2H]c1c([2H])c([2H])c2c(-c3ccc4ccc5ccccc5c4c3)c3c([2H])c([2H])c([2H])c([2H])c3c(-c3ccc4ccc(-c5cc6ccccc6c6c5oc5ccccc56)cc4c3)c2c1[2H]] | 65% | 705.90 |
| 16 | int 2-16 | A3-5 | 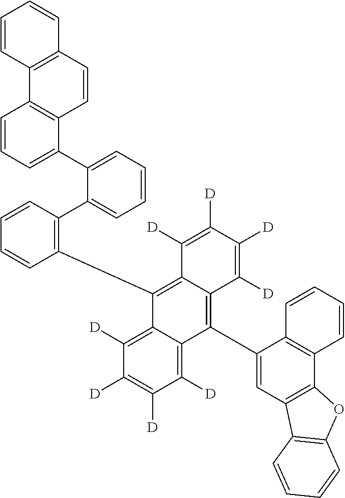 [SMILES: [2H]c1c([2H])c([2H])c2c(-c3cc4c5ccccc5oc4c4ccccc34)c3c([2H])c([2H])c([2H])c([2H])c3c(-c3ccccc3-c3ccccc3-c3cccc4c3ccc3ccccc34)c2c1[2H]] | 68% | 731.94 |
| 17 | int 2-17 | A3-6 | 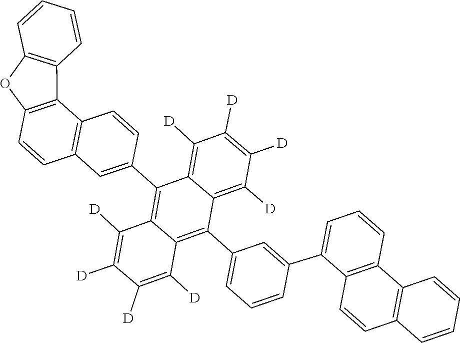 [SMILES: [2H]c1c([2H])c([2H])c2c(-c3ccc4c(ccc5oc6ccccc6c54)c3)c3c([2H])c([2H])c([2H])c([2H])c3c(-c3cccc(-c4cccc5c4ccc4ccccc45)c3)c2c1[2H]] | 63% | 655.84 |
| 18 | int 2-18 | A3-7 | 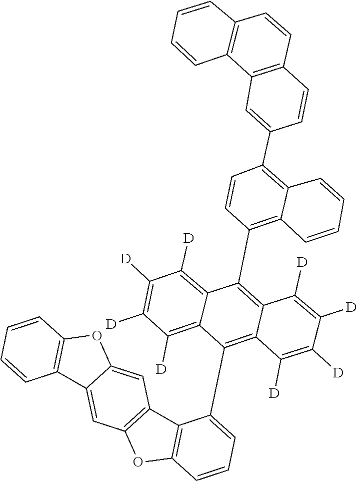 [SMILES: [2H]c1c([2H])c([2H])c2c(-c3cccc4oc5cc6c(cc5c34)oc3ccccc36)c3c([2H])c([2H])c([2H])c([2H])c3c(-c3ccc(-c4ccc5ccc6ccccc6c5c4)c4ccccc34)c2c1[2H]] | 61% | 745.92 |
| 19 | int 2-19 | 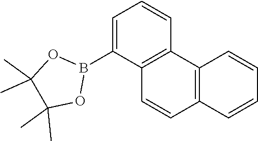 | 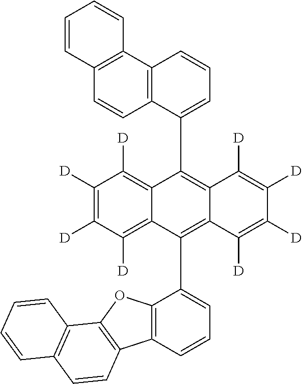 [SMILES: [2H]c1c([2H])c([2H])c2c(-c3cccc4c3oc3c5ccccc5ccc43)c3c([2H])c([2H])c([2H])c([2H])c3c(-c3cccc4c3ccc3ccccc34)c2c1[2H]] | 64% | 579.74 |
| 20 | int 2-20 | 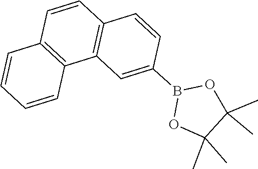 | 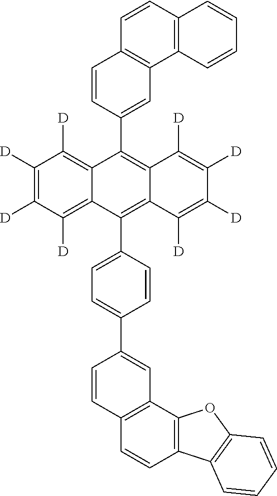 [SMILES: [2H]c1c([2H])c([2H])c2c(-c3ccc4ccc5ccccc5c4c3)c3c([2H])c([2H])c([2H])c([2H])c3c(-c3ccc(-c4ccc5ccc6c7ccccc7oc6c5c4)cc3)c2c1[2H]] | 60% | 655.84 |
| 21 | int 2-21 | A3-8 | 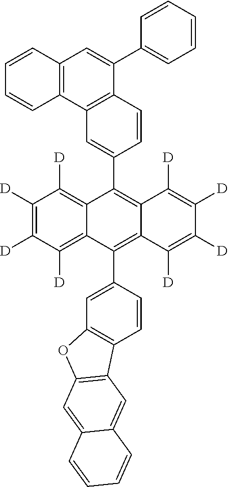 [SMILES: [2H]c1c([2H])c([2H])c2c(-c3ccc4c(-c5ccccc5)cc5ccccc5c4c3)c3c([2H])c([2H])c([2H])c([2H])c3c(-c3ccc4c(c3)oc3cc5ccccc5cc34)c2c1[2H]] | 68% | 655.84 |
| 22 | int 2-22 | A3-9 | 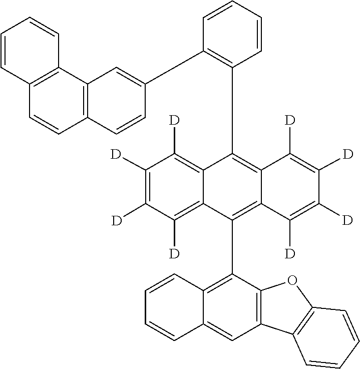 [SMILES: [2H]c1c([2H])c([2H])c2c(-c3c4ccccc4cc4c3oc3ccccc34)c3c([2H])c([2H])c([2H])c([2H])c3c(-c3ccccc3-c3ccc4ccc5ccccc5c4c3)c2c1[2H]] | 63% | 655.84 |
| 23 | int 2-23 | 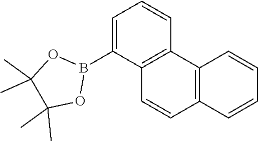 | 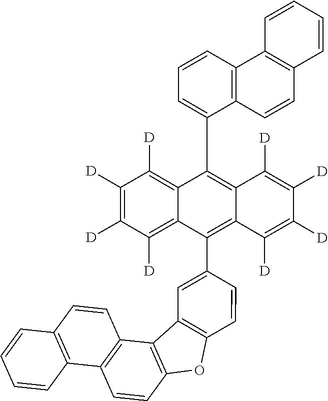 [SMILES: [2H]c1c([2H])c([2H])c2c(-c3cccc4c3ccc3ccccc34)c3c([2H])c([2H])c([2H])c([2H])c3c(-c3ccc4oc5ccc6c7ccccc7ccc6c5c4c3)c2c1[2H]] | 65% | 629.80 |
| 24 | int 2-24 | A3-10 | 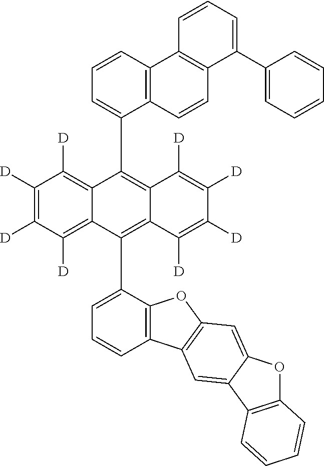 [SMILES: [2H]c1c([2H])c([2H])c2c(-c3cccc4c3oc3cc5oc6ccccc6c5cc34)c3c([2H])c([2H])c([2H])c([2H])c3c(-c3cccc4c3ccc3c(-c5ccccc5)cccc34)c2c1[2H]] | 66% | 695.86 |
| 25 | int 2-25 | 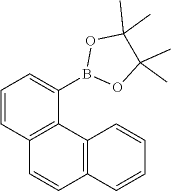 | 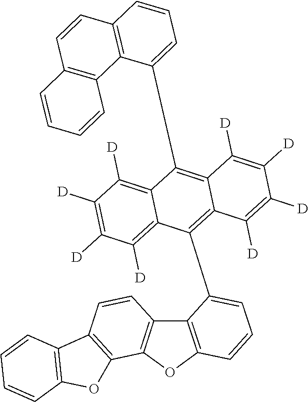 [SMILES: [2H]c1c([2H])c([2H])c2c(-c3cccc4oc5c(ccc6c7ccccc7oc65)c34)c3c([2H])c([2H])c([2H])c([2H])c3c(-c3cccc4ccc5ccccc5c34)c2c1[2H]] | 68% | 619.76 |
| 26 | int 2-26 | 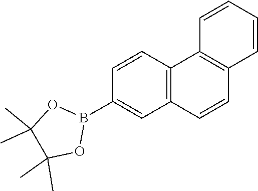 | 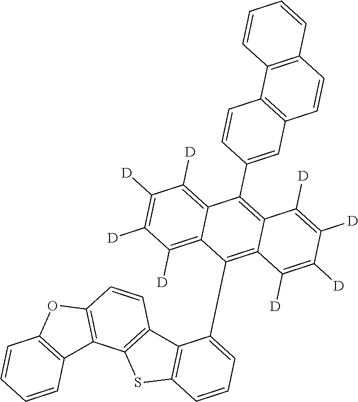 [SMILES: [2H]c1c([2H])c([2H])c2c(-c3cccc4sc5c(ccc6oc7ccccc7c65)c34)c3c([2H])c([2H])c([2H])c([2H])c3c(-c3ccc4c(ccc5ccccc54)c3)c2c1[2H]] | 60% | 635.82 |
| 27 | int 2-27 | A3-2 | 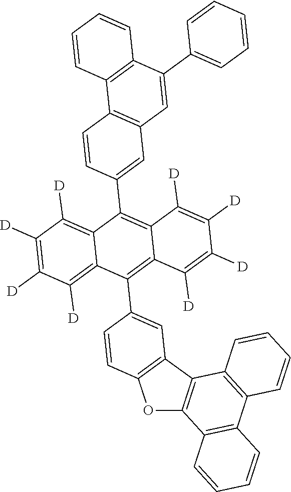 [SMILES: [2H]c1c([2H])c([2H])c2c(-c3ccc4oc5c6ccccc6c6ccccc6c5c4c3)c3c([2H])c([2H])c([2H])c([2H])c3c(-c3ccc4c(c3)cc(-c3ccccc3)c3ccccc34)c2c1[2H]] | 64% | 705.90 |

### 7) Synthesis of Chemical Formula 1D

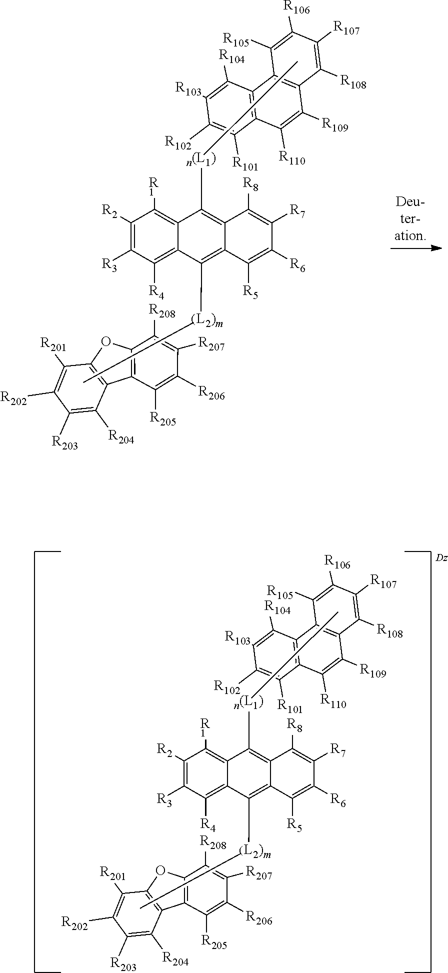

The reactant (1 eq) and trifluoromethanesulfonic acid (cat.) were put into C₆D₆ (10- to 50-fold mass ratio compared to the reactant), and the resulting mixture was stirred at 70° C. for 10 minutes to 100 minutes. After the reaction was completed, D₂O (excess) was added thereto, the resulting solution was stirred for 30 minutes, and then trimethylamine (excess) was added dropwise thereto. The reaction solution was transferred to a separatory funnel, and an extraction with water and chloroform was performed. The extract was dried over MgSO₄, and then recrystallized by heating with toluene to obtain the products (Compounds 28 to 31) of the following Table 10.

| | | | | | | Product | Number | | Ratio | Ratio of | |
|---|---|---|---|---|---|---|---|---|---|---|---|
| | | | | Product | Reaction | actual | of total | Number | of D | Total D | |
| | | | Reactant | theoretical | time | max | hydrogen | of D | substitutions | substitutions | |
| Compound | Reactant | Product | m/z | max m/z | (min) | mz | atoms | Solutions | (%) | (%) | Yield |
| 28 | Compund 5 | 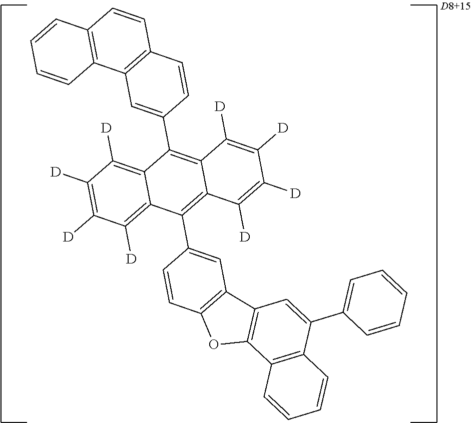 | 654 | 544 | 50 | 669 | 22 | 15 | 68% | 77% | 64% |
| 29 | Compound 12 | 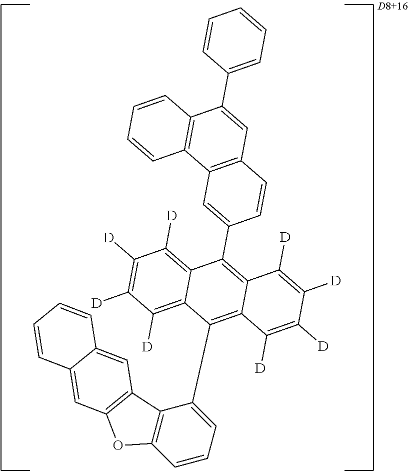 | 654 | 624 | 50 | 670 | 22 | 16 | 73% | 80% | 61% |
| 30 | Compound 17 | 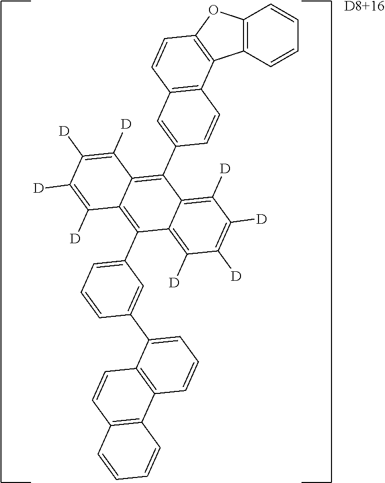 | 654 | 676 | 50 | 670 | 22 | 16 | 73% | 80% | 60% |
| 31 | Compound 25 | 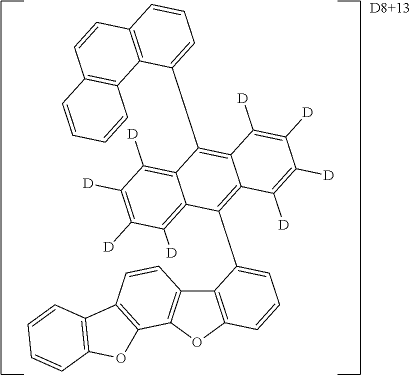 | 618 | 636 | 50 | 631 | 18 | 13 | 72% | 81% | 66% |

### [Each Product has a Different Degree of Deuterium Substitution Depending on the Reaction Time, and the Substitution Rate was Determined According to the Maximum m/z (M+) Value]

### <Example 1> Manufacture of OLED

As a positive electrode, a substrate on which ITO/Ag/ITO were deposited to have a thickness of 70 Å/1,000 Å/70 Å was cut into a size of 50 mm×50 mm×0.5 mm, put into distilled water in which a dispersant was dissolved, and washed with ultrasonic waves. A product manufactured by Fischer Co., was used as the detergent, and distilled water twice filtered using a filter manufactured by Millipore Co., was used as the distilled water. After the ITO was washed for 30 minutes, ultrasonic washing was conducted twice repeatedly using distilled water for 10 minutes. After the washing using distilled water was completed, ultrasonic washing was conducted using isopropyl alcohol, acetone, and methanol solvents in this order, and drying was then conducted.

HI-1 was thermally vacuum-deposited to have a thickness of 50 Å on the positive electrode thus prepared, thereby forming a hole injection layer, and HT1 as a material which transports holes was vacuum-deposited to have a thickness of 1,150 Å thereon, thereby forming a hole transport layer. Next, a hole adjusting layer was formed using EB1 (150 Å). Next, the host compound 1 synthesized in Preparation Example 1 and a dopant BD1 (2 wt %) were vacuum-deposited to have a thickness of 360 Å, thereby forming a light emitting layer. Thereafter, HB1 was deposited to have a thickness of 50 Å, thereby forming an electron adjusting layer, and Compounds ET1 and Liq were mixed at 5:5 (mass ratio), thereby forming an electron transport layer having a thickness of 250 Å. Sequentially, magnesium and lithium fluoride (LiF) were deposited to have a thickness of 50 Å to form a film as an electron injection layer <EIL>, magnesium and silver (1:4) were used to form a negative electrode having a thickness of 200 Å, and then CP1 was deposited to have a thickness of 600 Å, thereby completing a device. In the aforementioned procedure, the deposition rates of the organic materials were each maintained at 1 Å/sec.

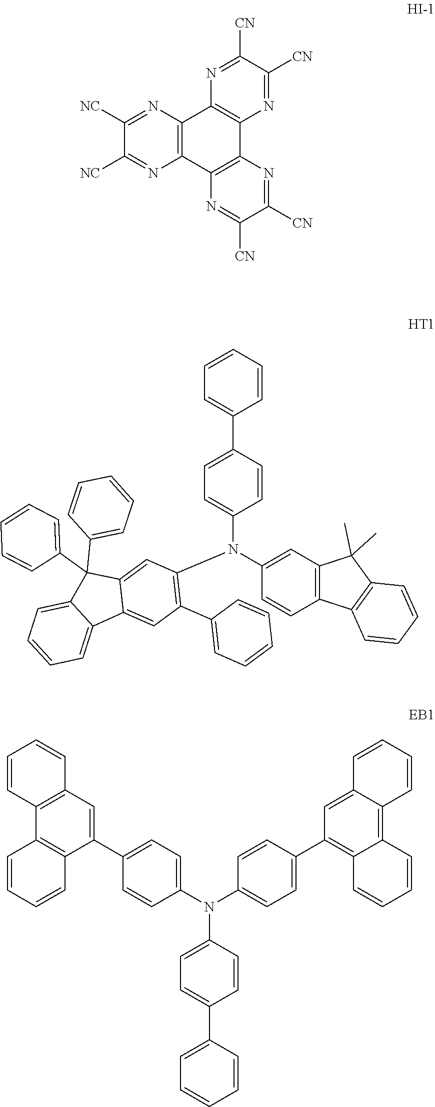 [SMILES: N#Cc1nc2c3nc(C#N)c(C#N)nc3c3nc(C#N)c(C#N)nc3c2nc1C#N ; CC1(C)c2ccccc2-c2ccc(N(c3ccc(-c4ccccc4)cc3)c3cc4c(cc3-c3ccccc3)-c3ccccc3C4(c3ccccc3)c3ccccc3)cc21 ; c1ccc(-c2ccc(N(c3ccc(-c4cc5ccccc5c5ccccc45)cc3)c3ccc(-c4cc5ccccc5c5ccccc45)cc3)cc2)cc1]

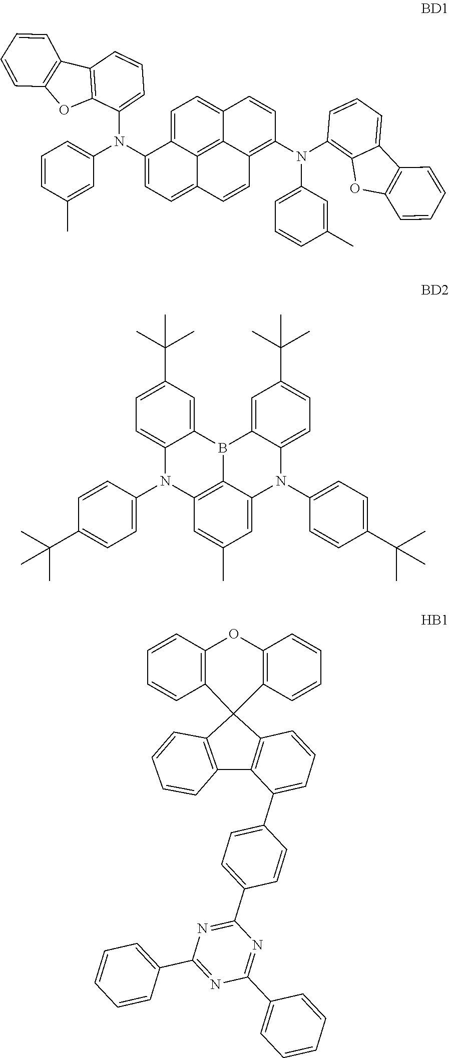 [SMILES: Cc1cccc(N(c2ccc3ccc4c(N(c5cccc(C)c5)c5cccc6c5oc5ccccc56)ccc5ccc2c3c54)c2cccc3c2oc2ccccc23)c1 ; Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1 ; c1ccc(-c2nc(-c3ccccc3)nc(-c3ccc(-c4cccc5c4-c4ccccc4C54c5ccccc5Oc5ccccc54)cc3)n2)cc1]

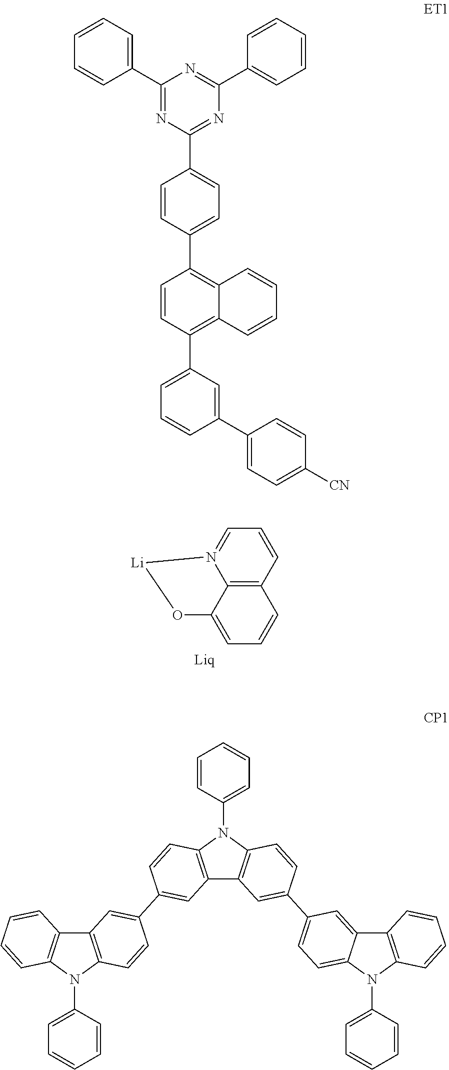

### Comparative Examples 1 to 17 and Examples 1 to 35

Devices were manufactured in the same manner as in Example 1, except that in Comparative Examples 1 to 17 and Examples 1 to 35, the materials shown in the following Table 11 were used as materials for the light emitting layer.

For the devices manufactured in Comparative Examples 1 to 17 and Examples 1 to 35, the driving voltage, light emitting efficiency, and a time (LT95) for reaching a 95% value compared to the initial luminance were measured at a current density of 20 mA/cm². The results are shown in the following Table 11.

TABLE 11

| | | | Device results | | | |
|---|---|---|---|---|---|---|
| | | | | | Color | Service life |
| Experimental | Light emitting layer | | Voltage (V) | Cd/A | coordinate | (T95, h) |
| Example | Host | Dopant | (@20mA/cm²) | (@20mA/cm²) | (x,y) | (@20mA/cm²) |
| Comparative | BH1 | BD1 | 3.65 | 5.44 | (0.140, | 51.6 |
| Example 1 | | | | | 0.053) | |
| Comparative | BH2 | BD1 | 3.73 | 5.28 | (0.140, | 53.0 |
| Example 2 | | | | | 0.055) | |
| Comparative | BH3 | BD1 | 3.67 | 5.40 | (0.141, | 52.9 |
| Example 3 | | | | | 0.058) | |
| Comparative | BH4 | BD1 | 3.63 | 5.43 | (0.142, | 50.3 |
| Example 4 | | | | | 0.073) | |
| Comparative | BH5 | BD1 | 3.60 | 5.35 | (0.142, | 54.2 |
| Example 5 | | | | | 0.071) | |
| Comparative | BH6 | BD1 | 3.64 | 5.38 | (0.140, | 53.1 |
| Example 6 | | | | | 0.078) | |
| Comparative | BH7 | BD1 | 3.65 | 5.40 | (0.140, | 52.3 |
| Example 7 | | | | | 0.078) | |
| Comparative | BH8 | BD1 | 3.61 | 5.31 | (0.142, | 51.6 |
| Example 8 | | | | | 0.073) | |
| Comparative | BH9 | BD1 | 3.58 | 5.48 | (0.140, | 46.7 |
| Example 9 | | | | | 0.072) | |
| Comparative | BH10 | BD1 | 3.60 | 5.43 | (0.140, | 47.8 |
| Example 10 | | | | | 0.076) | |
| Comparative | BH11 | BD1 | 3.61 | 5.44 | (0.140, | 48.3 |
| Example 11 | | | | | 0.075) | |
| Comparative | BH1 | BD2 | 3.70 | 5.44 | (0.132, | 47.3 |
| Example 14 | | | | | 0.057) | |
| Comparative | BH2 | BD2 | 3.78 | 5.28 | (0.132, | 48.8 |
| Example 15 | | | | | 0.056) | |
| Comparative | BH6 | BD2 | 3.69 | 5.38 | (0.133, | 47.4 |
| Example 16 | | | | | 0.081) | |
| Comparative | BH9 | BD2 | 3.64 | 5.48 | (0.133, | 43.0 |
| Example 17 | | | | | 0.079) | |
| Example 1 | Compound | BD1 | 3.49 | 5.69 | (0.140, | 61.3 |
| | 1 | | | | 0.054) | |
| Example 2 | Compound | BD1 | 3.36 | 5.73 | (0.140, | 60.9 |
| | 2 | | | | 0.053) | |
| Example 3 | Compound | BD1 | 3.39 | 5.70 | (0.140, | 63.8 |
| | 3 | | | | 0.053) | |
| Example 4 | Compound | BD1 | 3.43 | 5.75 | (0.141, | 65.0 |
| | 4 | | | | 0.065) | |
| Example 5 | Compound | BD1 | 3.47 | 5.78 | (0.140, | 62.5 |
| | 5 | | | | 0.055) | |
| Example 6 | Compound | BD1 | 3.41 | 5.73 | (0.141, | 67.5 |
| | 6 | | | | 0.063) | |
| Example 7 | Compound | BD1 | 3.38 | 5.76 | (0.141, | 65.4 |
| | 7 | | | | 0.068) | |
| Example 8 | Compound | BD1 | 3.40 | 5.80 | (0.140, | 68.1 |
| | 8 | | | | 0.058) | |
| Example 9 | Compound | BD1 | 3.42 | 5.72 | (0.140, | 63.9 |
| | 9 | | | | 0.055) | |
| Example 10 | Compound | BD1 | 3.36 | 5.77 | (0.140, | 64.2 |
| | 10 | | | | 0.065) | |
| Example 11 | Compound | BD1 | 3.40 | 5.80 | (0.141, | 63.0 |
| | 11 | | | | 0.055) | |
| Example 12 | Compound | BD1 | 3.38 | 5.79 | (0.140, | 64.9 |
| | 12 | | | | 0.055) | |
| Example 13 | Compound | BD1 | 3.43 | 5.74 | (0.140, | 65.5 |
| | 13 | | | | 0.056) | |
| Example 14 | Compound | BD1 | 3.37 | 5.72 | (0.141, | 60.1 |
| | 14 | | | | 0.065) | |
| Example 15 | Compound | BD1 | 3.39 | 5.70 | (0.140, | 68.4 |
| | 15 | | | | 0.061) | |
| Example 16 | Compound | BD1 | 3.42 | 5.73 | (0.140, | 68.0 |
| | 16 | | | | 0.059) | |
| Example 17 | Compound | BD1 | 3.39 | 5.74 | (0.141, | 65.7 |
| | 17 | | | | 0.059) | |
| Example 18 | Compound | BD1 | 3.35 | 5.70 | (0.140, | 66.9 |
| | 18 | | | | 0.062) | |
| Example 19 | Compound | BD1 | 3.48 | 5.75 | (0.140, | 65.0 |
| | 19 | | | | 0.053) | |
| Example 20 | Compound | BD1 | 3.39 | 5.71 | (0.140, | 62.7 |
| | 20 | | | | 0.060) | |
| Example 21 | Compound | BD1 | 3.41 | 5.69 | (0.141, | 66.6 |
| | 21 | | | | 0.059) | |
| Example 22 | Compound | BD1 | 3.40 | 5.72 | (0.141, | 64.5 |
| | 22 | | | | 0.059) | |
| Example 23 | Compound | BD1 | 3.37 | 5.73 | (0.140, | 68.1 |
| | 23 | | | | 0.063) | |
| Example 24 | Compound | BD1 | 3.38 | 5.70 | (0.140, | 63.4 |
| | 24 | | | | 0.065) | |
| Example 25 | Compound | BD1 | 3.38 | 5.78 | (0.140, | 62.0 |
| | 25 | | | | 0.063) | |
| Example 26 | Compound | BD1 | 3.3.6 | 5.82 | (0.140, | 64.1 |
| | 26 | | | | 0.063) | |
| Example 27 | Compound | BD1 | 3.40 | 5.79 | (0.140, | 63.0 |
| | 27 | | | | 0.065) | |
| Example 28 | Compound | BD1 | 3.48 | 5.78 | (0.140, | 70.1 |
| | 28 | | | | 0.055) | |
| Example 29 | Compound | BD1 | 3.38 | 5.80 | (0.140, | 73.4 |
| | 29 | | | | 0.055) | |
| Example 30 | Compound | BD1 | 3.40 | 5.73 | (0.141, | 77.1 |
| | 30 | | | | 0.059) | |
| Example 31 | Compound | BD1 | 3.38 | 5.78 | (0.140, | 69.3 |
| | 31 | | | | 0.063) | |
| Example 32 | Compound | BD2 | 3.55 | 5.88 | (0.132, | 62.8 |
| | 28 | | | | 0.058) | |
| Example 33 | Compound | BD2 | 3.46 | 5.88 | (0.132, | 65.4 |
| | 29 | | | | 0.057) | |
| Example 34 | Compound | BD2 | 3.45 | 5.81 | (0.133, | 69.9 |
| | 30 | | | | 0.063) | |
| Example 35 | Compound | BD2 | 3.44 | 5.86 | (0.133, | 60.8 |
| | 31 | | | | 0.068) | |

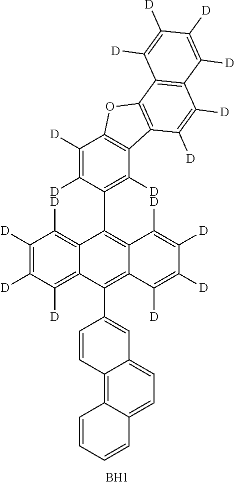

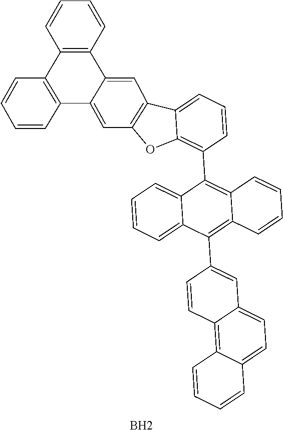

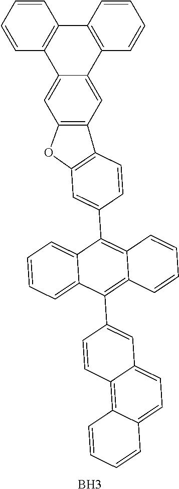

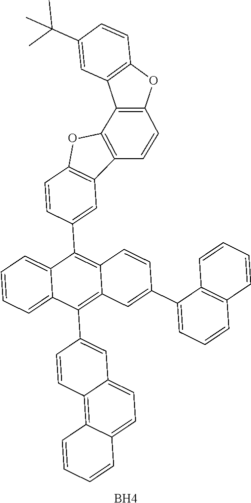

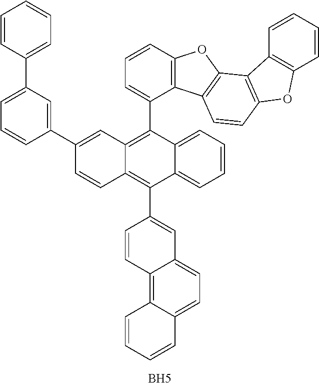

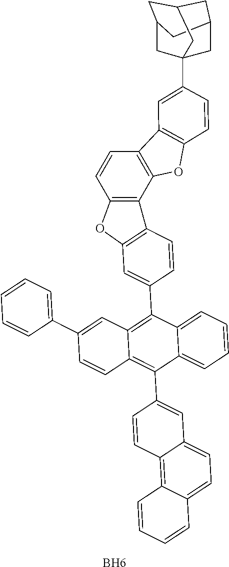

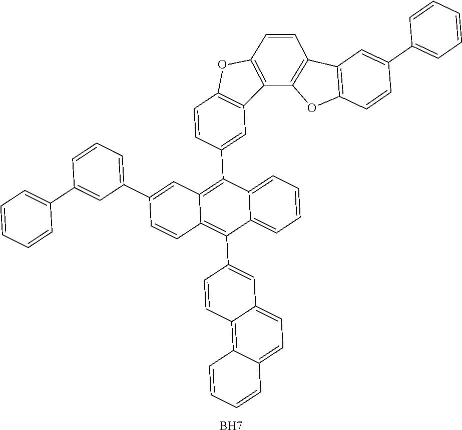

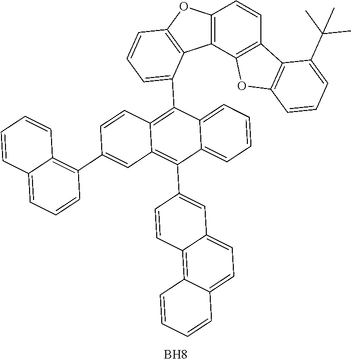

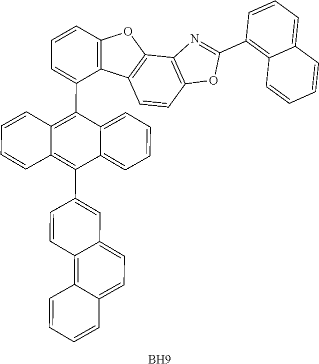

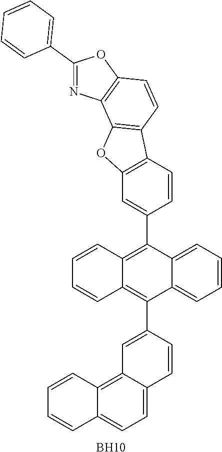

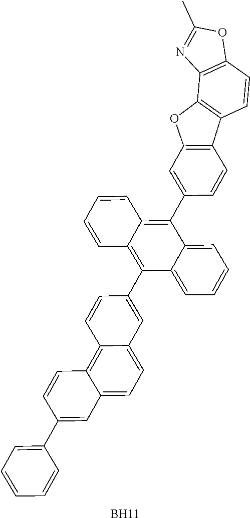 [SMILES: Cc1nc2c(ccc3c4ccc(-c5c6ccccc6c(-c6ccc7c(ccc8cc(-c9ccccc9)ccc87)c6)c6ccccc56)cc4oc32)o1 ; "BH11"]

The results in Table 11 above are for the case of having a device structure in which a light emitting layer is formed, in which the host of the light emitting layer is the sole host, and in which the compound in the claims of the present application is composed of the host of the light emitting layer.

Comparative Example 1 is a case in which the 2nd carbon of a phenanthrene is bonded to No. 10 position of an anthracene and the 2nd carbon of an [a]naphthobenzofuran is bonded to No. 9 position of an anthracene, with deuterium being introduced into the anthracene and the [a]naphthobenzofuran, and differs from the present application in that additional substituents are introduced into the phenanthrene or [a]naphthobenzofuran. As described above, in Example 9 or Example 11, an additional aryl group is substituted, which results in different carrier injection and migration speeds from those in the Comparative Example, so that it can be confirmed that the present device structure shows excellent performance, particularly in terms of efficiency.

Comparative Examples 2 and 3 are cases of having a skeleton in which the 2nd carbon of a phenanthrene is bonded to No. 10 position of an anthracene, and the 1st or 2nd carbon of a phenanthrenodibenzofuran is bonded to No. 9 position of the anthracene, and these also differ from the present application in the presence or absence of additional substituents. From the results of Comparative Example 1, Example 9, or Example 11, as it can be seen that the difference in the bonding position of phenanthrene or the introduction of a substituent into the skeleton as in Example 23 or Example 27 affects the stability and electrical characteristics, the excellent device performance in the above devices can be confirmed.

Comparative Examples 4 to 8 have the structures of the claims of the present application, but have an additional substituent introduced at the 2nd or 3rd carbon of an anthracene, and have a common structure in which the 2nd carbon of the phenanthrene is bonded to the 10th carbon of the anthracene, and the corresponding skeletal structures lengthen the color coordinate at the bond of the 2nd carbon of the phenanthrene, and show a deterioration in overall electrical characteristics.

Comparative Examples 9 to 11 have a structure in which an oxazole-condensed dibenzofuran core is bonded to the 9th carbon of the anthracene, and phenanthrene is introduced into the 10th carbon, unlike the skeleton of the present application, and Comparative Examples 9 to 11 show lower voltage characteristics than the previous Comparative Examples 1 to 8, but are inferior in terms of efficiency and service life, which is more noticeable when compared with Examples 1 to 35.

In Examples 1 to 27 of the present application, it can be seen that the performance of the device has been secured while applying the compound corresponding to Chemical Formula 1 as a host in the light emitting layer to grow a role of injecting holes and making a balance as a carrier, and maintaining low voltage characteristics and efficiency which are considered to be currently important in a blue light emitting device.

In Examples 1 to 27, the compounds of the claims are hosts for the light emitting layer, and the phenanthrene and naphthobenzofuran or heteroaryl-condensed dibenzofuran (including S) units are substituted with additional aryl groups to increase the efficiency and improve the stability of the device, so that the effect of deuterium in anthracene is maximized, thereby showing additional service life extension.

In addition, Examples 28 to 31 show that additional service life improvement effect can be achieved despite the cost disadvantage while showing an additional service life increase of 10 to 15% on average through additional deuterium substitution, and Examples 32 to 35 can show the device consistency when various dopants are introduced because changing the dopant (pyrene dopant->boron dopant) maintains the tendency of the device while maintaining the change of the dopant characteristics.

### <Example 36> Manufacture of OLED

As a positive electrode, a substrate on which ITO/Ag/ITO were deposited to have a thickness of 70 Å/1,000 Å/70 Å was cut into a size of 50 mm×50 mm×0.5 mm, put into distilled water in which a dispersant was dissolved, and washed with ultrasonic waves. A product manufactured by Fischer Co., was used as the detergent, and distilled water twice filtered using a filter manufactured by Millipore Co., was used as the distilled water. After the ITO was washed for 30 minutes, ultrasonic washing was conducted twice repeatedly using distilled water for 10 minutes. After the washing using distilled water was completed, ultrasonic washing was conducted using isopropyl alcohol, acetone, and methanol solvents in this order, and drying was then conducted.

HI-1 was thermally vacuum-deposited to have a thickness of 50 Å on the positive electrode thus prepared, thereby forming a hole injection layer, and HT1 as a material which transports holes was vacuum-deposited to have a thickness of 1,150 Å thereon, thereby forming a hole transport layer. Next, a hole adjusting layer was formed using EB1 (150 Å). Next, as a light emitting layer, the host compound 5 synthesized in Preparation Example 1, BH13, and a dopant BD1 (2 wt %) were vacuum-co-deposited to have a thickness of 360 Å, thereby forming a light emitting layer. Thereafter, HB1 was deposited to have a thickness of 50 Å, thereby forming an electron adjusting layer, and Compounds ET1 and Liq were mixed at 5:5 (mass ratio), thereby forming an electron transport layer having a thickness of 250 Å. Sequentially, magnesium and lithium fluoride (LiF) were deposited to have a thickness of 50 Å to form a film as an electron injection layer <EIL>, magnesium and silver (1:4) were used to form a negative electrode having a thickness of 200 Å, and then CP1 was deposited to have a thickness of 600 Å, thereby completing a device. In the aforementioned procedure, the deposition rates of the organic materials were each maintained at 1 Å/sec.

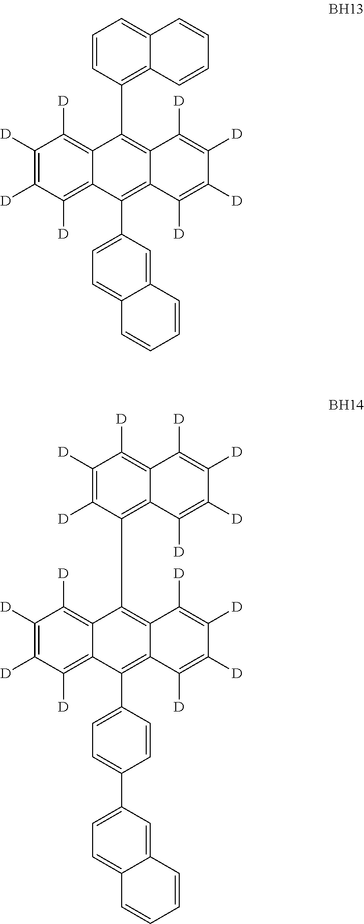 [SMILES: [2H]c1c([2H])c([2H])c2c(-c3cccc4ccccc34)c3c([2H])c([2H])c([2H])c([2H])c3c(-c3ccc4ccccc4c3)c2c1[2H] ; [2H]c1c([2H])c([2H])c2c(-c3c4c([2H])c([2H])c([2H])c([2H])c4c(-c4ccc(-c5ccc6ccccc6c5)cc4)c4c([2H])c([2H])c([2H])c([2H])c34)c([2H])c([2H])c([2H])c2c1[2H]]

### Comparative Examples 18 to 23 and Examples 36 to 40

Devices were manufactured in the same manner as in Example 27, except that in Comparative Examples 18 to 23 and Examples 36 to 40, the materials shown in the following Table 2 were used as materials for the light emitting layer.

However, the host of the light emitting layer is an embodiment in which two hosts as shown in Table 12 were co-deposited using other evaporation sources during device manufacture to form a light emitting layer.

For the devices manufactured in Comparative Examples 18 to 23 and Examples 36 to 40, the driving voltage, light emitting efficiency, and a time (LT95) for reaching a 95% value compared to the initial luminance were measured at a current density of 20 mA/cm². The results are shown in the following [Table 12].

TABLE 12

| | Light emitting layer | | | | | |
|---|---|---|---|---|---|---|
| | First host:host | | Device results | | | |
| | Second | | | | Color | Service life |
| Experimental | (Ratio, mixing | | Voltage (V) | Cd/A | coordinate | (T95, h) |
| Example | method) | Dopant | (@20 mA/cm²) | (@20 mA/cm²) | (x, y) | (@20 mA/cm²) |
| Comparative | BH1:BH13 | BD1 | 3.57 | 5.64 | (0.140, | 53.4 |
| Example 18 | (1:1) | | | | 0.050) | |
| Comparative | BH2:BH14 | BD1 | 3.68 | 5.36 | (0.140, | 55.9 |
| Example 19 | (1:1) | | | | 0.053) | |
| Comparative | BH5:BH13 | BD1 | 3.52 | 5.41 | (0.141, | 56.8 |
| Example 20 | (1:1) | | | | 0.068) | |
| Comparative | BH9:BH14 | BD1 | 3.50 | 5.62 | (0.140, | 51.2 |
| Example 21 | (1:1) | | | | 0.070) | |
| Comparative | BH1:BH13 | BD2 | 3.64 | 5.73 | (0.140, | 48.1 |
| Example 22 | (1:1) | | | | 0.050) | |
| Comparative | BH9:BH14 | BD2 | 3.56 | 5.65 | (0.140, | 46.2 |
| Example 23 | (1:1) | | | | 0.070) | |
| Example 36 | Compound5:BH13 | BD1 | 3.39 | 5.90 | (0.140, | 78.6 |
| | (1:1) | | | | 0.052) | |
| Example 37 | Compound12:BH13 | BD1 | 3.31 | 5.91 | (0.139, | 80.9 |
| | (1:1) | | | | 0.051) | |
| Example 38 | Compound17:BH14 | BD1 | 3.33 | 5.88 | (0.141, | 88.4 |
| | (1:1) | | | | 0.055) | |
| Example 39 | Compound25:BH14 | BD1 | 3.30 | 5.92 | (0.140, | 80.1 |
| | (1:1) | | | | 0.060) | |
| Example 40 | Compound25:BH14 | BD2 | 3.39 | 6.05 | (0.133, | 72.6 |
| | (1:1) | | | | 0.057) | |

The results in [Table 12]above are for the case where the host in the light emitting layer is composed of a mixed host (a mixed host of two anthracene species).

Comparative Examples 18 to 21 show examples in which mixed hosts were formed using BH13 among widely used aryl-based anthracene compounds and Comparative 14, and BH1, BH2, BH5, and BH9 among the structures of Comparative Examples 1 to 12, as comparative examples, and it was observed that devices having light emitting layers formed using the corresponding combinations showed device results that were partially improved in all aspects of voltage, efficiency, and service life. Furthermore, Comparative Examples 22 and 23 show the same tendency even when boron-based blue dopants are applied.

In Examples 36 to 40, Compounds 28 to 31 among the example compounds of the claims of the present application were mixed with the previous BH13 or BH14 to form mixed hosts, and the device results were observed, and it can be seen that the compounds of the claims of the present application, which had excellent characteristics compared to Comparative Examples 1 to 17, have the same tendency even under mixed host conditions.

What is claimed is:

1. An organic compound of the following Chemical Formula 1: 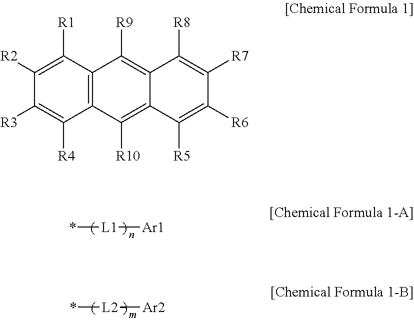
wherein, in Chemical Formula 1,
one or more of R1 to R10 are bonded to L1 in Chemical Formula 1-A above;
one or more of R1 to R10 not bonded to L1 in Chemical Formula 1-A above are bonded to L2 in Chemical Formula 1-B above,
R1 to R10, which are not bonded to L1 in Chemical Formula 1-A above and are not bonded to L2 in Chemical Formula 1-B above, are the same as or different from each other, and are each independently hydrogen; deuterium; a substituted or unsubstituted alkyl group; a substituted or unsubstituted cycloalkyl group; a substituted or unsubstituted aryl group; or a substituted or unsubstituted heteroaryl group,
L1 and L2 are the same as or different from each other, and are each independently a direct bond; a substituted or unsubstituted arylene group; or a substituted or unsubstituted heteroarylene group,
n and m are each an integer from 1 to 4,
Ar1 is the following Chemical Formula 1-C, 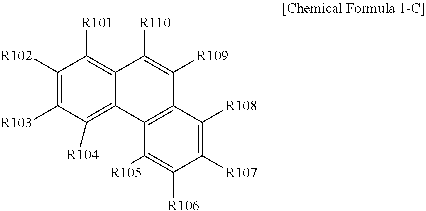
in Chemical Formula 1-C,
R101 to R110 are the same as or different from each other, and are each independently hydrogen; deuterium; a substituted or unsubstituted alkyl group; a substituted or unsubstituted cycloalkyl group; a substituted or unsubstituted aryl group; or a substituted or unsubstituted heteroaryl group, and one of R101 to R108 is bonded to L1,
Ar2 is the following Chemical Formula 1-D, 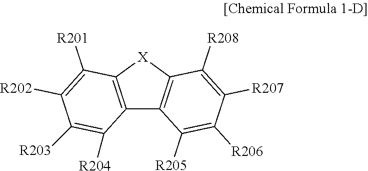
in Chemical Formula 1-D,
X is O or S,
R201 to R208 are the same as or different from each other, and are each independently hydrogen; deuterium; a substituted or unsubstituted alkyl group; a substituted or unsubstituted cycloalkyl group; a substituted or unsubstituted aryl group; or a substituted or unsubstituted heteroaryl group, and at least one pair adjacent to each other in R201 to R208 is bonded to each other to necessarily form one or more substituted or unsubstituted ring groups,
the substituted or unsubstituted ring group is a substituted or unsubstituted aromatic hydrocarbon ring group; a substituted or unsubstituted benzofuran ring group; or a substituted or unsubstituted benzothiophene ring group,
the substituted or unsubstituted ring group; or any one of R201 to R208 not forming the substituted or unsubstituted ring group is bonded to L2,
provided that when adjacent R201 to R208 are bonded to each other to form a substituted or unsubstituted aromatic hydrocarbon ring group, any one of R101, R103 to R106, and R108 in Chemical Formula 1-C is bonded to L1, or any one of R101 to R108, which are not bonded to L1 in Chemical Formula 1-C, and R201 to R208, which are not bonded to L2 in Chemical Formula 1-D, is necessarily one or more of a substituted or unsubstituted alkyl group, a substituted or unsubstituted cycloalkyl group, or a substituted or unsubstituted aryl group, or the substituted or unsubstituted ring group, which is not bonded to L2 in Chemical Formula 1-D, is substituted with a substituted or unsubstituted alkyl group, a substituted or unsubstituted cycloalkyl group, or a substituted or unsubstituted aryl group, and
when adjacent R201 to R208 are bonded to each other to form a substituted or unsubstituted benzofuran ring group or a substituted or unsubstituted benzothiophene ring group, R2, R3, R6, and R7 in Chemical Formula 1 are the same as or different from each other, and are each independently hydrogen or deuterium, and
* is a moiety bonded to Chemical Formula 1.

2. The organic compound of claim 1, wherein the substituted or unsubstituted ring groups of Chemical Formula 1-D are represented by the following 1-D-1 to 1-D-9: 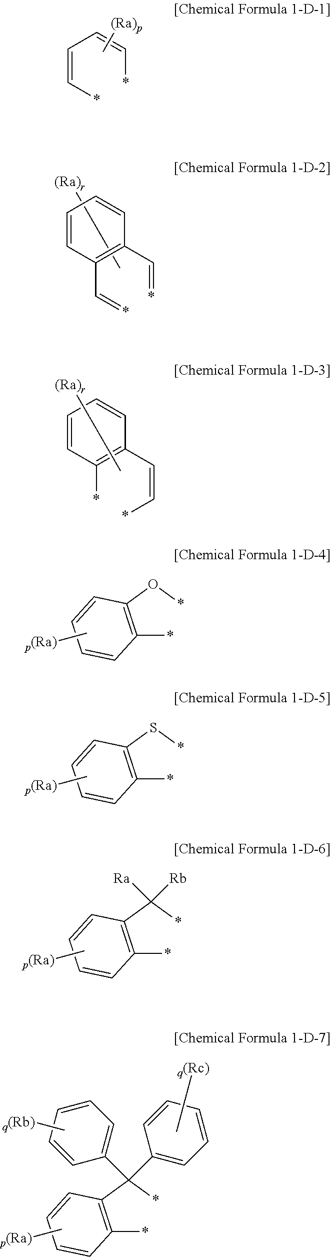 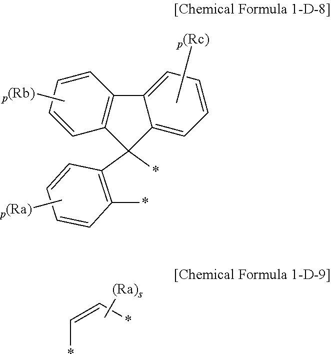
in Chemical Formulae 1-D-1 to 1-D-9,
Ra to Rc are the same as or different from each other, and are each independently hydrogen; deuterium; a substituted or unsubstituted alkyl group; a substituted or unsubstituted aryl group; or a substituted or unsubstituted heterocyclic group, or optionally bonded to an adjacent group to form a substituted or unsubstituted ring group, or bonded to L2 above,
p is an integer from 1 to 4, and when p is 2 or higher, two or more Ra's, Rb's, or Rc's are the same as or different from each other,
q is an integer from 1 to 5, and when q is 2 or higher, two or more Rb's or Rc's are the same as or different from each other,
r is an integer from 1 to 6, and when r is 2 or higher, two or more Ra's are the same as or different from each other,
s is 1 or 2, and when s is 2, two Ra's are the same as or different from each other, and
* is a moiety bonded to an adjacent pair of R201 to R208 in Chemical Formula 1-D.

3. The organic compound of claim 1, wherein Chemical Formula 1 is any one of the following Chemical Formulae 1-1 to 1-4: 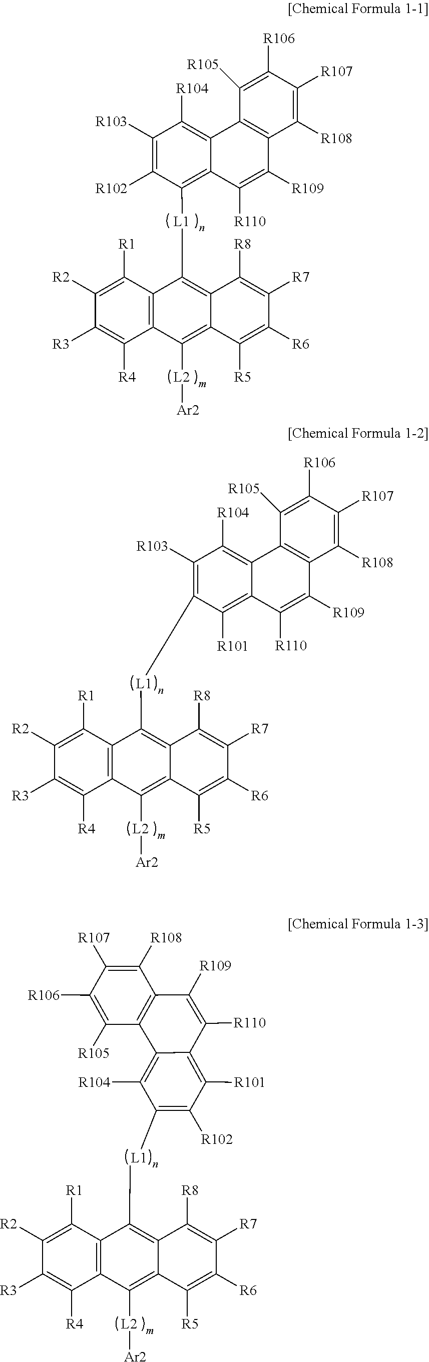 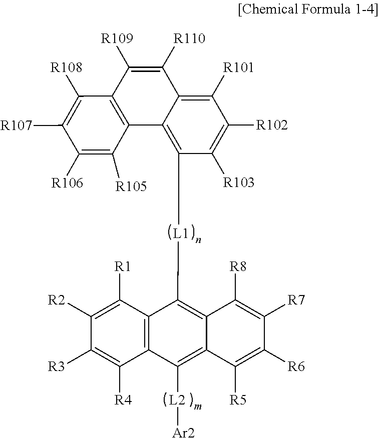
in Chemical Formulae 1-1 to 1-4,
the definitions of L1, L2, Ar2, R101 to R110, n, and m are the same as the definitions in Chemical Formula 1 above, and
R1 to R8 are hydrogen; deuterium; a substituted or unsubstituted alkyl group; a substituted or unsubstituted cycloalkyl group; a substituted or unsubstituted aryl group;
or a substituted or unsubstituted heteroaryl group.

4. The organic compound of claim 1, wherein Chemical Formula 1 is any one of the following Chemical Formulae 1-5 to 1-8: 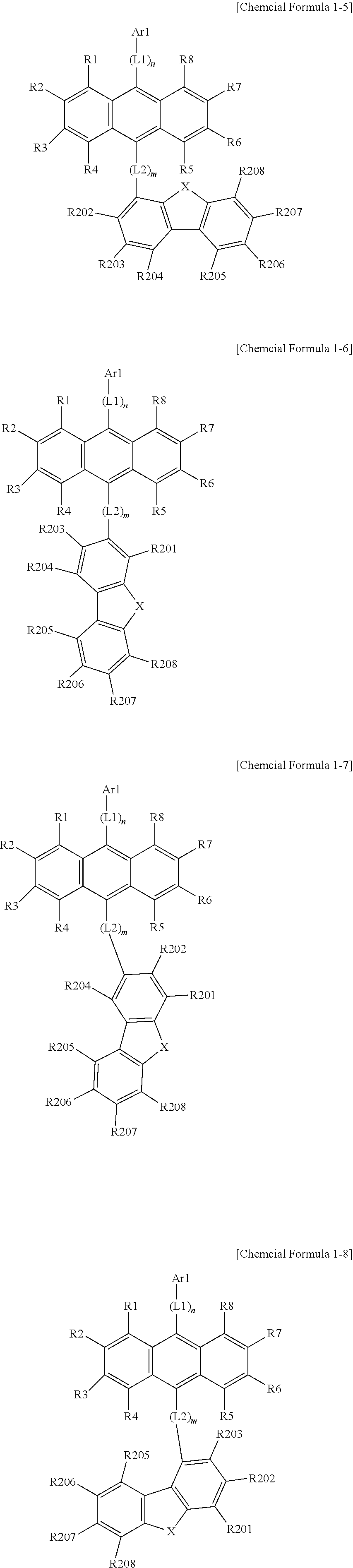
in Chemical Formulae 1-5 to 1-8,
the definitions of L1, L2, Ar1, X, R201 to R208, n, and m are the same as the definitions in Chemical Formula 1 above, and
R1 to R8 are hydrogen; deuterium; a substituted or unsubstituted alkyl group; a substituted or unsubstituted cycloalkyl group; a substituted or unsubstituted aryl group; or a substituted or unsubstituted heteroaryl group.

5. The organic compound of claim 1, wherein R9 of Chemical Formula 1 above is bonded to L1 of Chemical Formula 1-A above, and R10 of Chemical Formula 1 above is bonded to L2 of Chemical Formula 1-B above, and
all of R1 to R8 of Chemical Formula 1 above are deuterium.

6. The organic compound of claim 1, wherein R201 to R208, which are not bonded to L2, are the same as or different from each other, and are each independently hydrogen; or deuterium.

7. The organic compound of claim 1, wherein a deuterium substitution rate of Chemical Formula 1 above is 15% or more.

8. The organic compound of claim 1, wherein Chemical Formula 1 is any one of the following compounds:

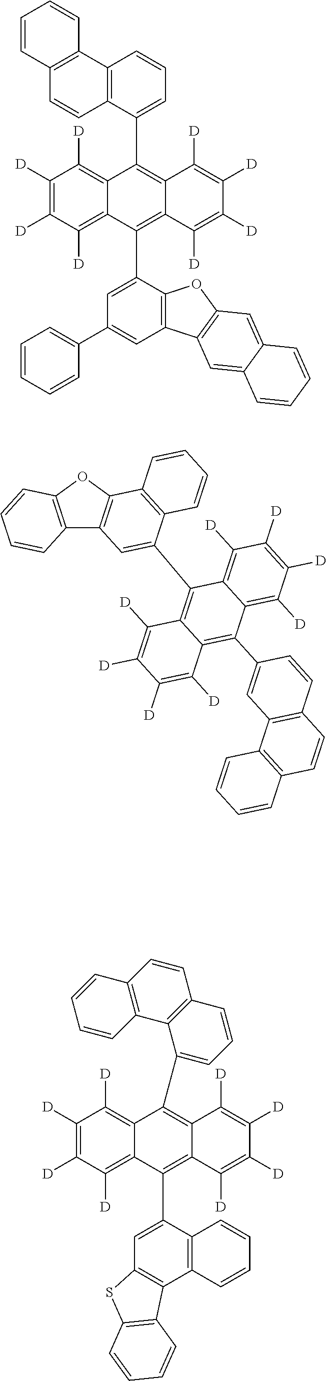 [SMILES: [2H]c1c([2H])c([2H])c2c(-c3cc(-c4ccccc4)cc4c3oc3cc5ccccc5cc34)c3c([2H])c([2H])c([2H])c([2H])c3c(-c3cccc4c3ccc3ccccc34)c2c1[2H] ; [2H]c1c([2H])c([2H])c2c(-c3cc4c5ccccc5oc4c4ccccc34)c3c([2H])c([2H])c([2H])c([2H])c3c(-c3ccc4ccc5ccccc5c4c3)c2c1[2H] ; [2H]c1c([2H])c([2H])c2c(-c3cccc4ccc5ccccc5c34)c3c([2H])c([2H])c([2H])c([2H])c3c(-c3cc4sc5ccccc5c4c4ccccc34)c2c1[2H]]

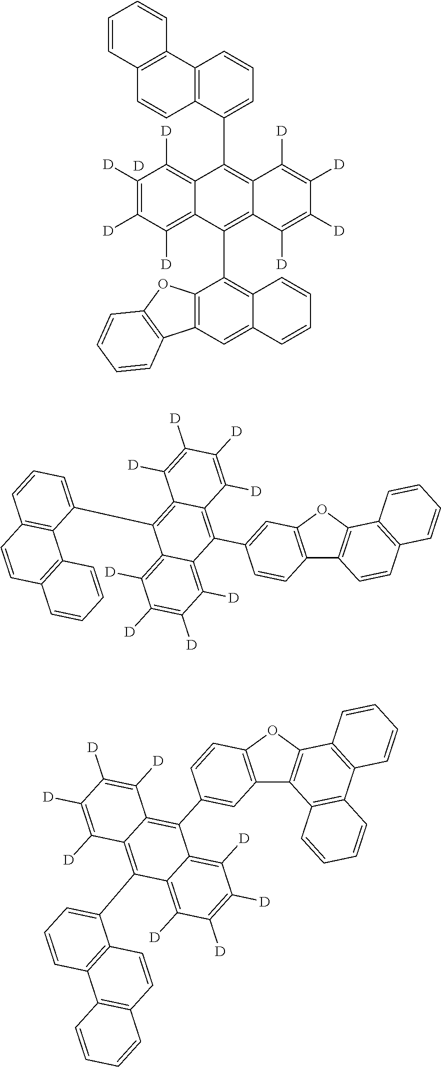

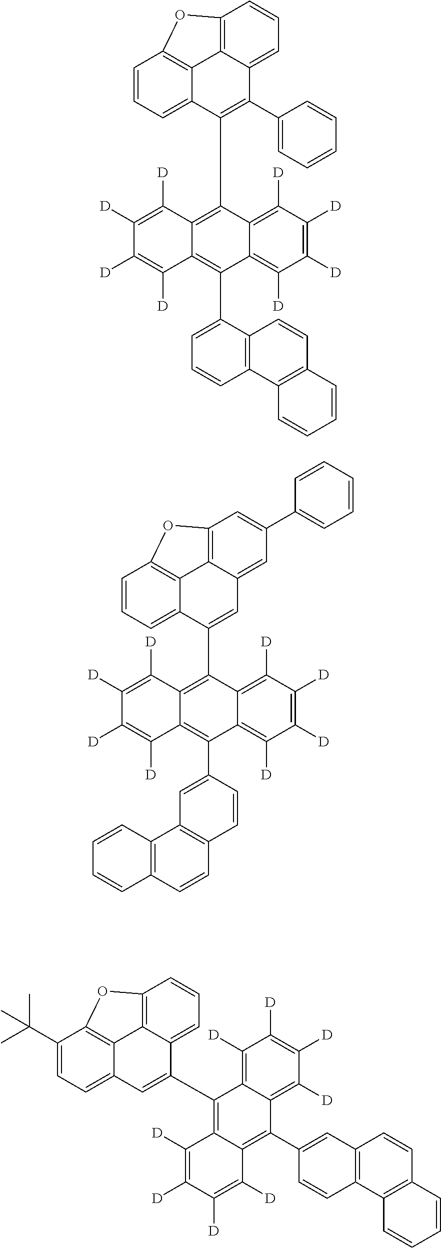

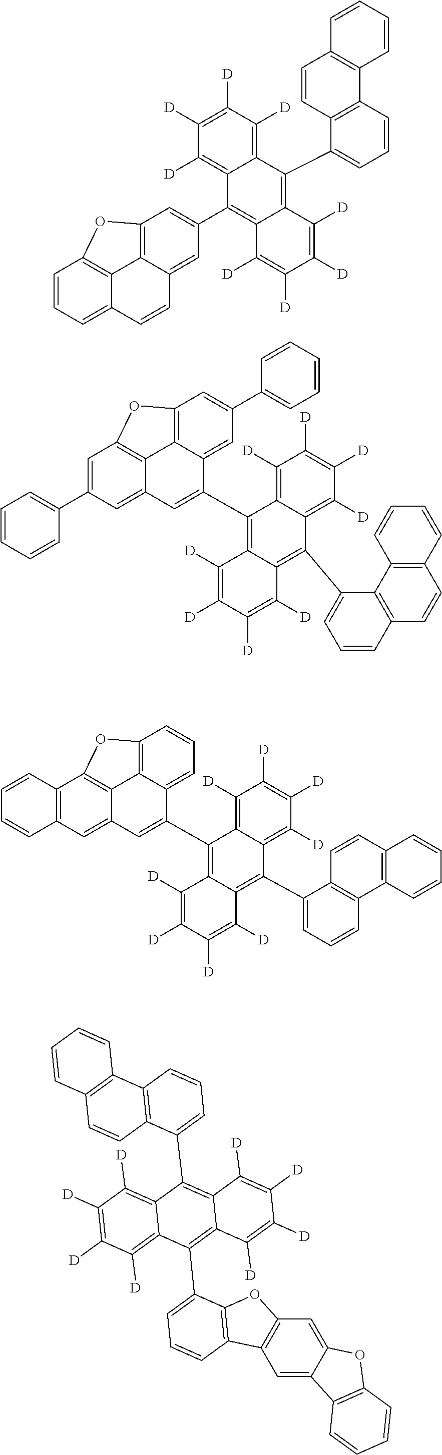

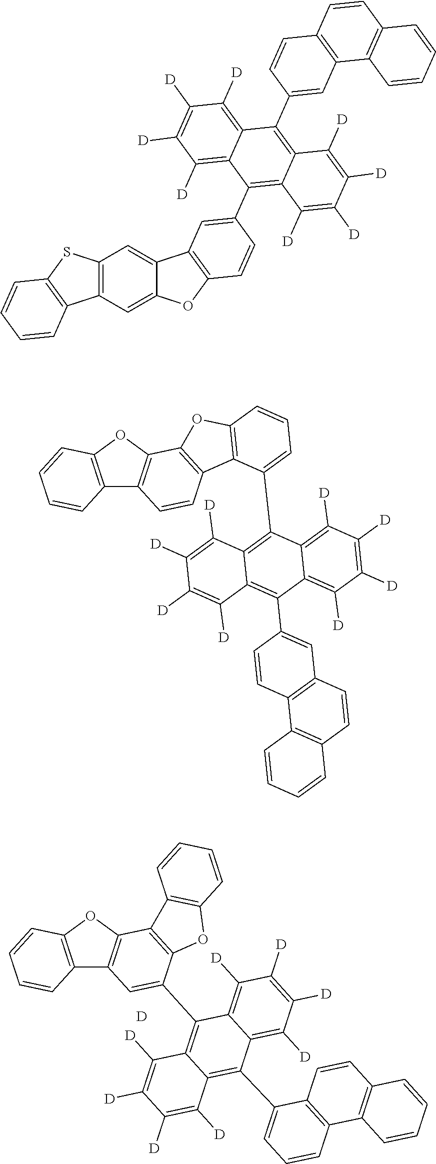

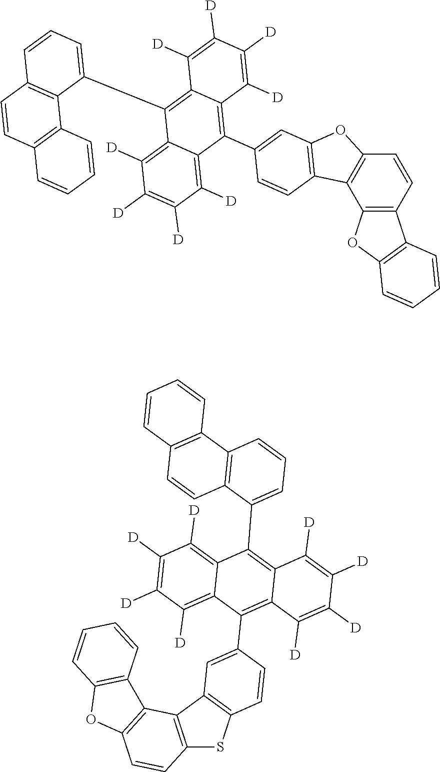 [SMILES: [2H]c1c([2H])c([2H])c2c(-c3cccc4ccc5ccccc5c34)c3c([2H])c([2H])c([2H])c([2H])c3c(-c3ccc4c(c3)oc3ccc5c6ccccc6oc5c34)c2c1[2H] ; [2H]c1c([2H])c([2H])c2c(-c3cccc4c3ccc3ccccc34)c3c([2H])c([2H])c([2H])c([2H])c3c(-c3ccc4sc5ccc6oc7ccccc7c6c5c4c3)c2c1[2H]]

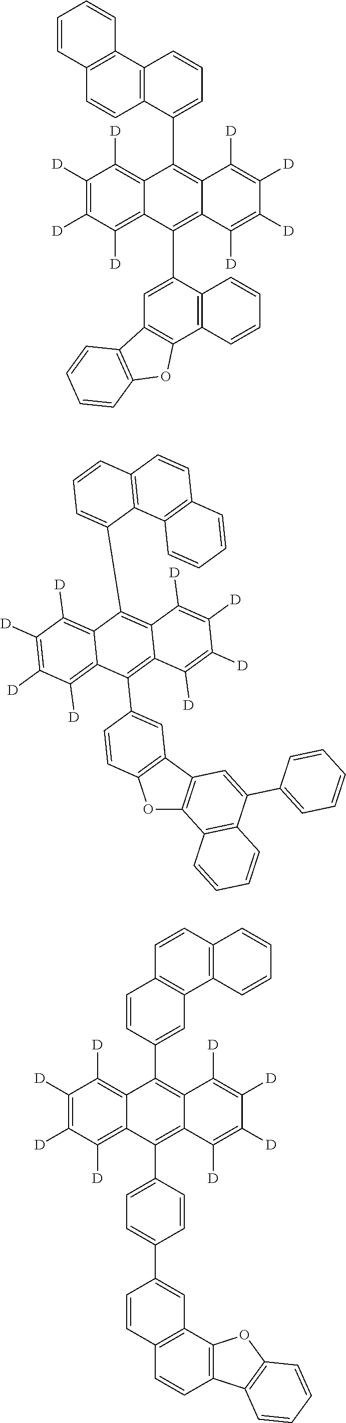

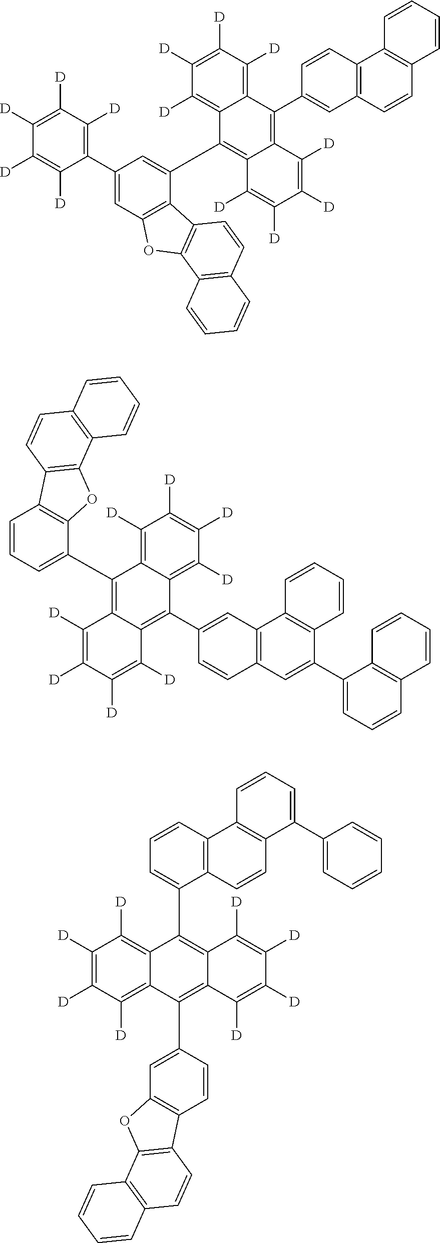 [SMILES: [2H]c1c([2H])c([2H])c(-c2cc(-c3c4c([2H])c([2H])c([2H])c([2H])c4c(-c4ccc5c(ccc6ccccc65)c4)c4c([2H])c([2H])c([2H])c([2H])c34)c3c(c2)oc2c4ccccc4ccc23)c([2H])c1[2H] ; [2H]c1c([2H])c([2H])c2c(-c3cccc4c3oc3c5ccccc5ccc43)c3c([2H])c([2H])c([2H])c([2H])c3c(-c3ccc4cc(-c5cccc6ccccc56)c5ccccc5c4c3)c2c1[2H] ; [2H]c1c([2H])c([2H])c2c(-c3cccc4c3ccc3c(-c5ccccc5)cccc34)c3c([2H])c([2H])c([2H])c([2H])c3c(-c3ccc4c(c3)oc3c5ccccc5ccc43)c2c1[2H]]

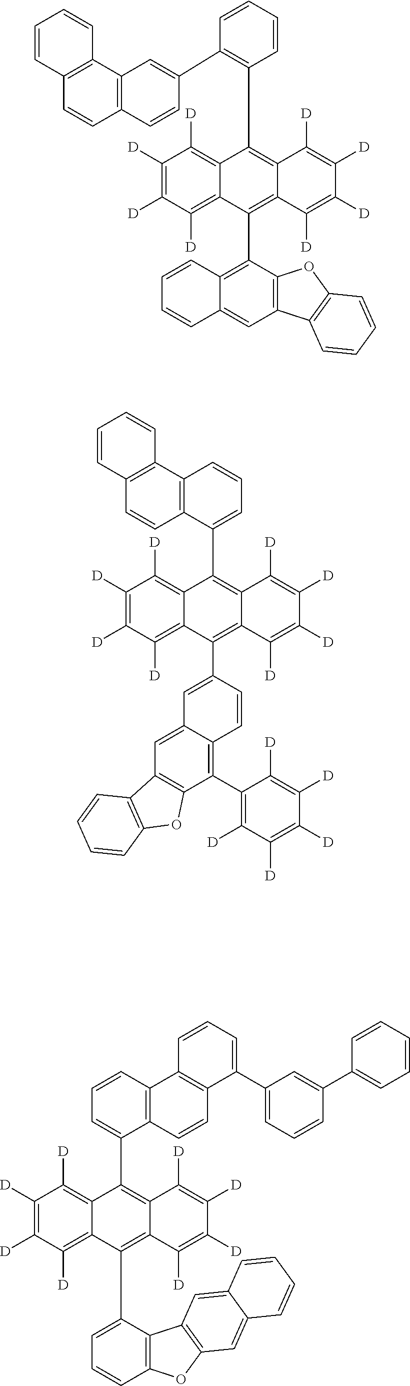 [SMILES: [2H]c1c([2H])c([2H])c2c(-c3c4ccccc4cc4c3oc3ccccc34)c3c([2H])c([2H])c([2H])c([2H])c3c(-c3ccccc3-c3ccc4ccc5ccccc5c4c3)c2c1[2H] ; [2H]c1c([2H])c([2H])c(-c2c3ccc(-c4c5c([2H])c([2H])c([2H])c([2H])c5c(-c5cccc6c5ccc5ccccc56)c5c([2H])c([2H])c([2H])c([2H])c45)cc3cc3c2oc2ccccc23)c([2H])c1[2H] ; [2H]c1c([2H])c([2H])c2c(-c3cccc4oc5cc6ccccc6cc5c34)c3c([2H])c([2H])c([2H])c([2H])c3c(-c3cccc4c3ccc3c(-c5cccc(-c6ccccc6)c5)cccc34)c2c1[2H]]

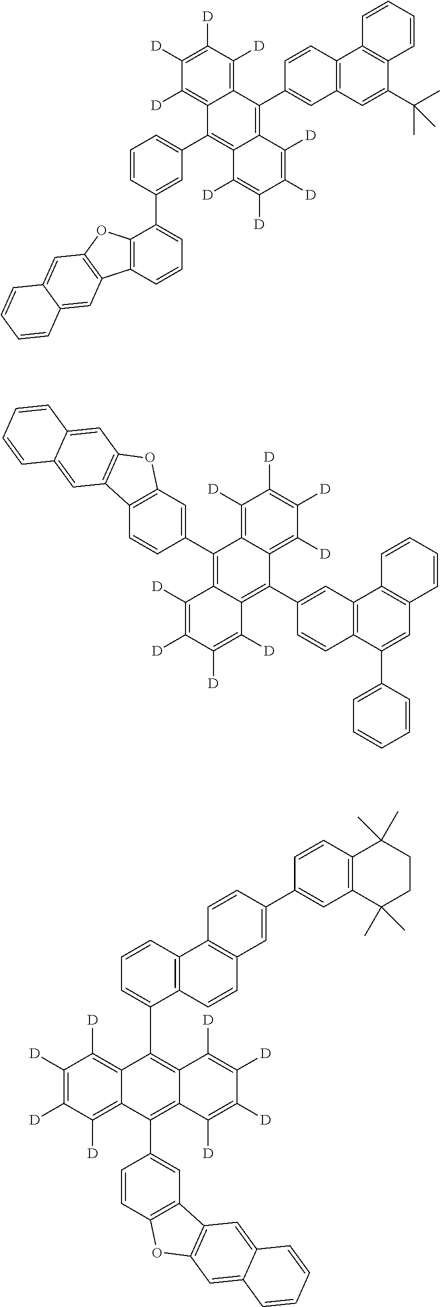 [SMILES: [2H]c1c([2H])c([2H])c2c(-c3ccc4c(c3)cc(C(C)(C)C)c3ccccc34)c3c([2H])c([2H])c([2H])c([2H])c3c(-c3cccc(-c4cccc5c4oc4cc6ccccc6cc45)c3)c2c1[2H] ; [2H]c1c([2H])c([2H])c2c(-c3ccc4c(-c5ccccc5)cc5ccccc5c4c3)c3c([2H])c([2H])c([2H])c([2H])c3c(-c3ccc4c(c3)oc3cc5ccccc5cc34)c2c1[2H] ; [2H]c1c([2H])c([2H])c2c(-c3cccc4c3ccc3cc(-c5ccc6c(c5)C(C)(C)CCC6(C)C)ccc34)c3c([2H])c([2H])c([2H])c([2H])c3c(-c3ccc4oc5cc6ccccc6cc5c4c3)c2c1[2H]]

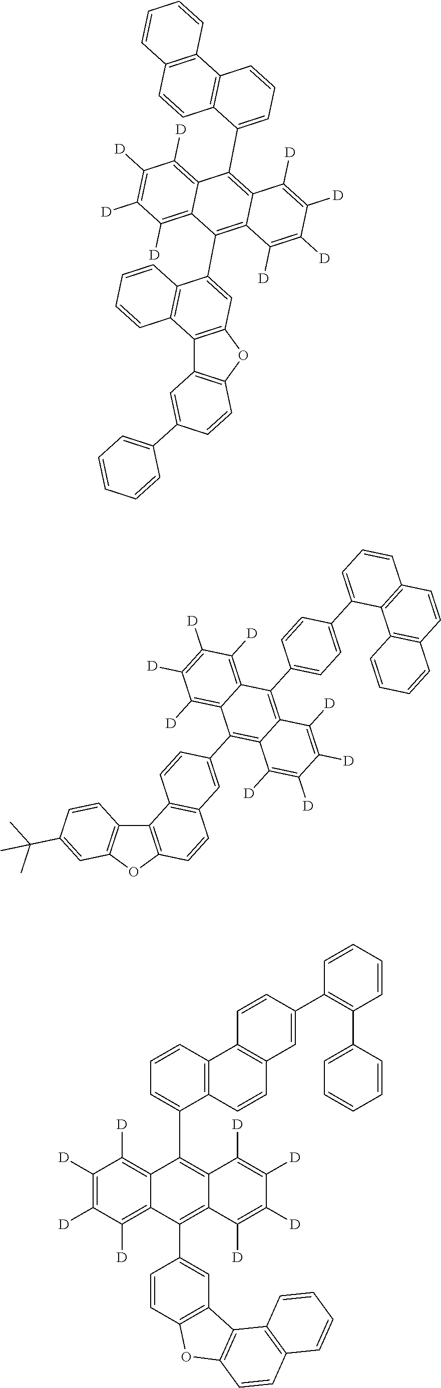 [SMILES: [2H]c1c([2H])c([2H])c2c(-c3cc4oc5ccc(-c6ccccc6)cc5c4c4ccccc34)c3c([2H])c([2H])c([2H])c([2H])c3c(-c3cccc4c3ccc3ccccc34)c2c1[2H] ; [2H]c1c([2H])c([2H])c2c(-c3ccc4c(ccc5oc6cc(C(C)(C)C)ccc6c54)c3)c3c([2H])c([2H])c([2H])c([2H])c3c(-c3ccc(-c4cccc5ccc6ccccc6c45)cc3)c2c1[2H] ; [2H]c1c([2H])c([2H])c2c(-c3cccc4c3ccc3cc(-c5ccccc5-c5ccccc5)ccc34)c3c([2H])c([2H])c([2H])c([2H])c3c(-c3ccc4oc5ccc6ccccc6c5c4c3)c2c1[2H]]

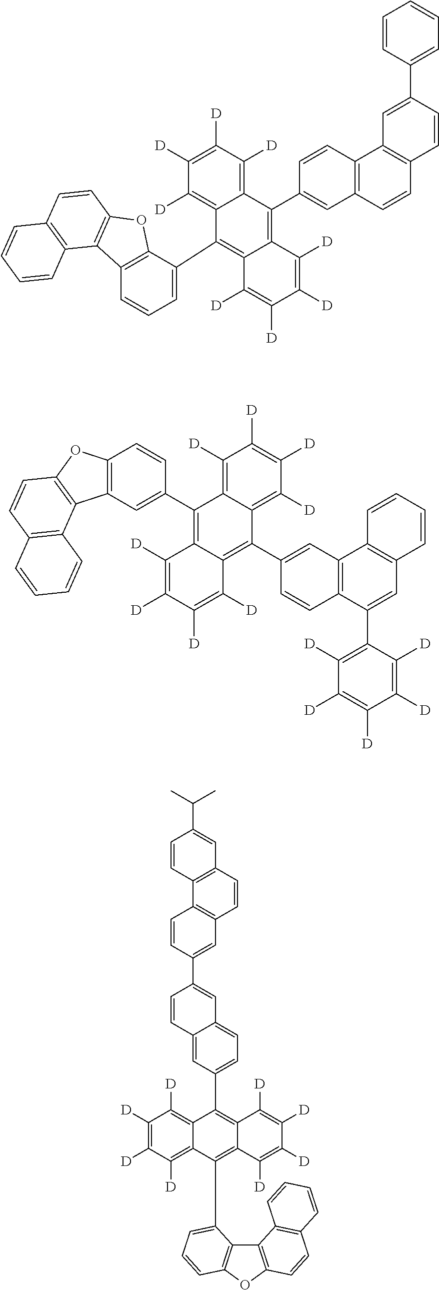 [SMILES: [2H]c1c([2H])c([2H])c2c(-c3cccc4c3oc3ccc5ccccc5c34)c3c([2H])c([2H])c([2H])c([2H])c3c(-c3ccc4c(ccc5ccc(-c6ccccc6)cc54)c3)c2c1[2H] ; [2H]c1c([2H])c([2H])c(-c2cc3ccccc3c3cc(-c4c5c([2H])c([2H])c([2H])c([2H])c5c(-c5ccc6oc7ccc8ccccc8c7c6c5)c5c([2H])c([2H])c([2H])c([2H])c45)ccc23)c([2H])c1[2H] ; [2H]c1c([2H])c([2H])c2c(-c3cccc4oc5ccc6ccccc6c5c34)c3c([2H])c([2H])c([2H])c([2H])c3c(-c3ccc4cc(-c5ccc6c(ccc7cc(C(C)C)ccc76)c5)ccc4c3)c2c1[2H]]

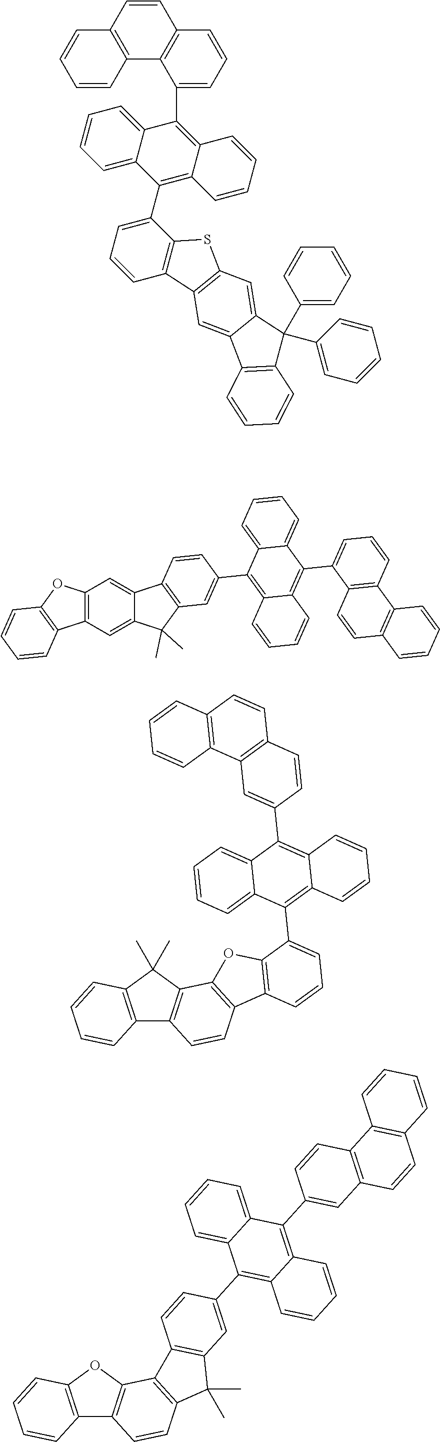 [SMILES: c1ccc(C2(c3ccccc3)c3ccccc3-c3cc4c(cc32)sc2c(-c3c5ccccc5c(-c5cccc6ccc7ccccc7c56)c5ccccc35)cccc24)cc1 ; CC1(C)c2cc(-c3c4ccccc4c(-c4cccc5c4ccc4ccccc45)c4ccccc34)ccc2-c2cc3oc4ccccc4c3cc21 ; CC1(C)c2ccccc2-c2ccc3c(oc4c(-c5c6ccccc6c(-c6ccc7ccc8ccccc8c7c6)c6ccccc56)cccc43)c21 ; CC1(C)c2cc(-c3c4ccccc4c(-c4ccc5c(ccc6ccccc65)c4)c4ccccc34)ccc2-c2c1ccc1c2oc2ccccc21]

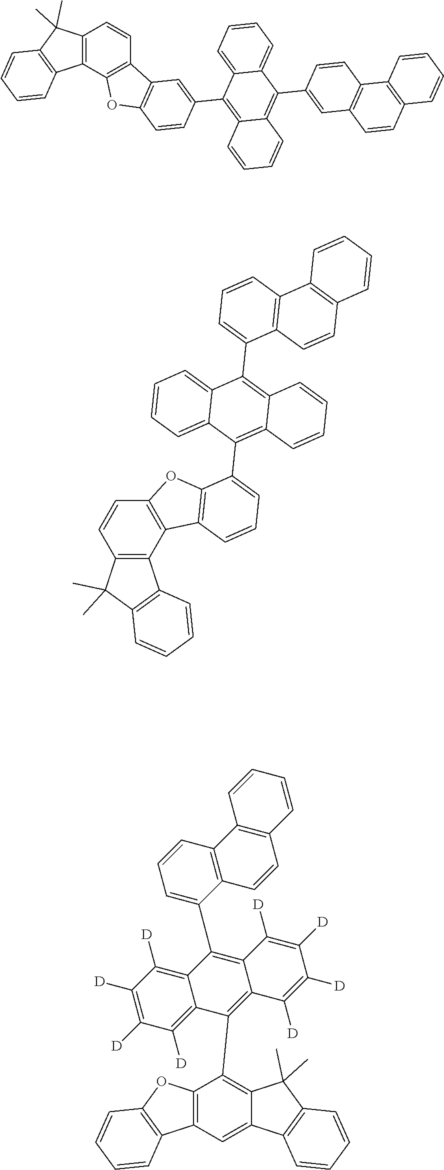 [SMILES: CC1(C)c2ccccc2-c2c1ccc1c2oc2ccc(-c3c4ccccc4c(-c4ccc5c(ccc6ccccc65)c4)c4ccccc34)cc21 ; CC1(C)c2ccccc2-c2c1ccc1oc3c(-c4c5ccccc5c(-c5cccc6c5ccc5ccccc56)c5ccccc45)cccc3c21 ; [2H]c1c([2H])c([2H])c2c(-c3c4c(cc5c3oc3ccccc35)-c3ccccc3C4(C)C)c3c([2H])c([2H])c([2H])c([2H])c3c(-c3cccc4c3ccc3ccccc34)c2c1[2H]]

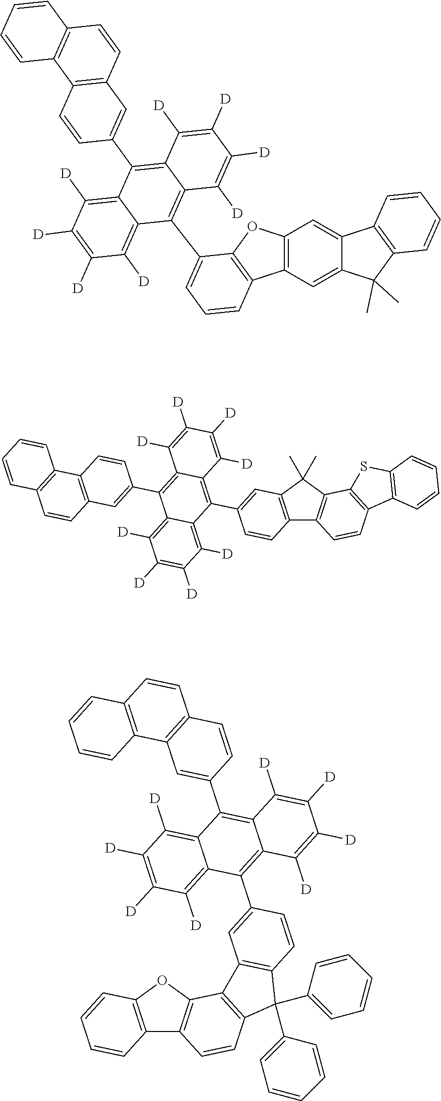 [SMILES: [2H]c1c([2H])c([2H])c2c(-c3cccc4c3oc3cc5c(cc34)C(C)(C)c3ccccc3-5)c3c([2H])c([2H])c([2H])c([2H])c3c(-c3ccc4c(ccc5ccccc54)c3)c2c1[2H] ; [2H]c1c([2H])c([2H])c2c(-c3ccc4c(ccc5ccccc54)c3)c3c([2H])c([2H])c([2H])c([2H])c3c(-c3ccc4c(c3)C(C)(C)c3c-4ccc4c3sc3ccccc34)c2c1[2H] ; [2H]c1c([2H])c([2H])c2c(-c3ccc4ccc5ccccc5c4c3)c3c([2H])c([2H])c([2H])c([2H])c3c(-c3ccc4c(c3)-c3c(ccc5c3oc3ccccc35)C4(c3ccccc3)c3ccccc3)c2c1[2H]]

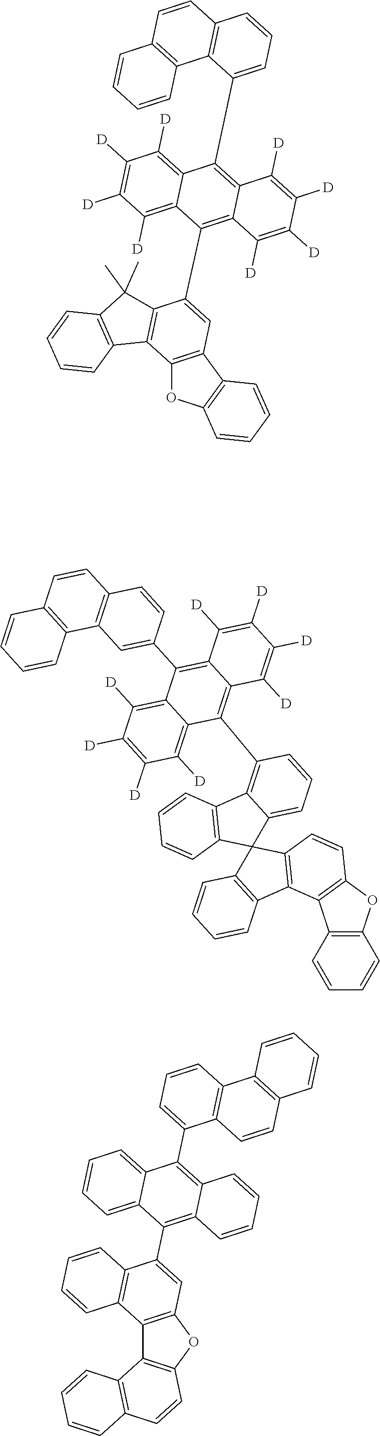 [SMILES: [2H]c1c([2H])c([2H])c2c(-c3cccc4ccc5ccccc5c34)c3c([2H])c([2H])c([2H])c([2H])c3c(-c3cc4c(oc5ccccc54)c4c3C(C)(C)c3ccccc3-4)c2c1[2H] ; [2H]c1c([2H])c([2H])c2c(-c3cccc4c3-c3ccccc3C43c4ccccc4-c4c3ccc3oc5ccccc5c43)c3c([2H])c([2H])c([2H])c([2H])c3c(-c3ccc4ccc5ccccc5c4c3)c2c1[2H] ; c1ccc2c(c1)ccc1c(-c3c4ccccc4c(-c4cc5oc6ccc7ccccc7c6c5c5ccccc45)c4ccccc34)cccc12]

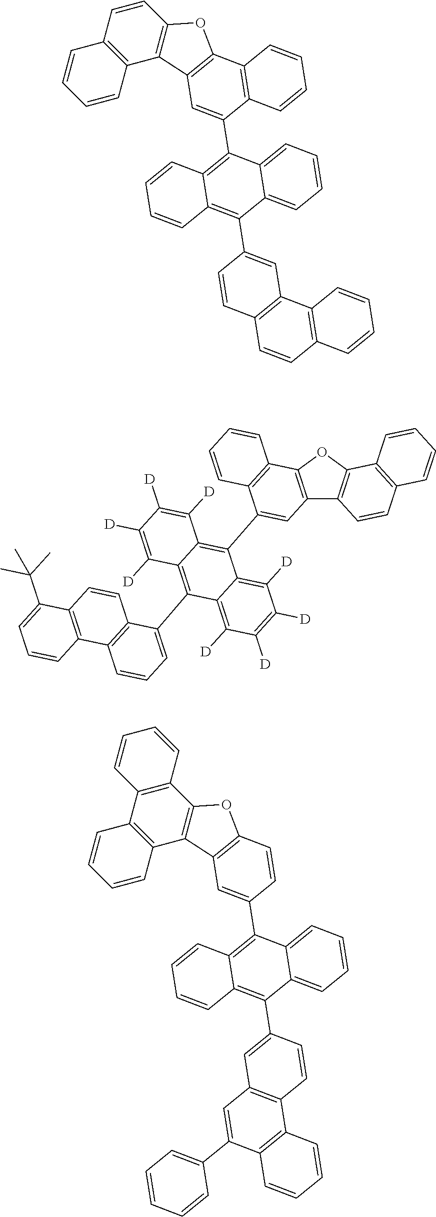 [SMILES: c1ccc2c(c1)ccc1ccc(-c3c4ccccc4c(-c4cc5c(oc6ccc7ccccc7c65)c5ccccc45)c4ccccc34)cc12 ; [2H]c1c([2H])c([2H])c2c(-c3cc4c5ccc6ccccc6c5oc4c4ccccc34)c3c([2H])c([2H])c([2H])c([2H])c3c(-c3cccc4c3ccc3c(C(C)(C)C)cccc34)c2c1[2H] ; c1ccc(-c2cc3cc(-c4c5ccccc5c(-c5ccc6oc7c8ccccc8c8ccccc8c7c6c5)c5ccccc45)ccc3c3ccccc23)cc1]

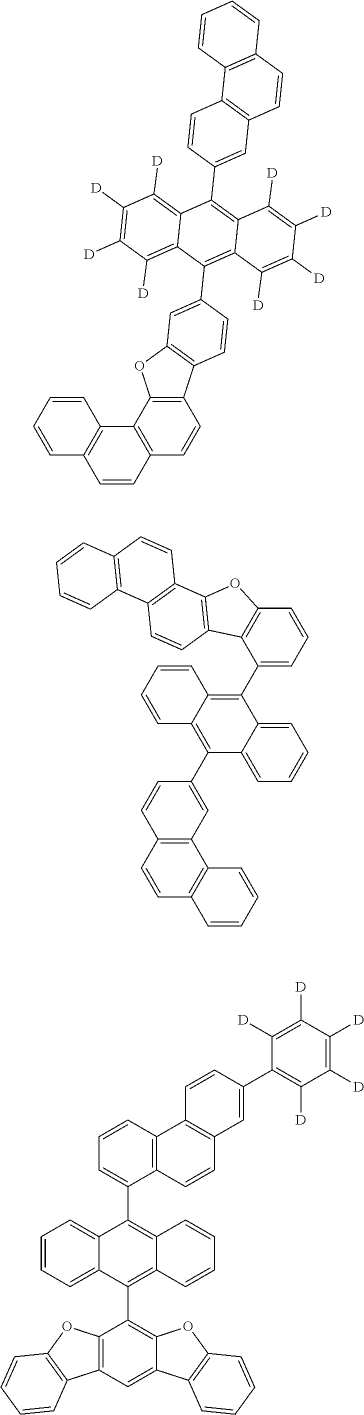 [SMILES: [2H]c1c([2H])c([2H])c2c(-c3ccc4c(c3)oc3c4ccc4ccc5ccccc5c43)c3c([2H])c([2H])c([2H])c([2H])c3c(-c3ccc4c(ccc5ccccc54)c3)c2c1[2H] ; c1ccc2c(c1)ccc1ccc(-c3c4ccccc4c(-c4cccc5oc6c7ccc8ccccc8c7ccc6c45)c4ccccc34)cc12 ; [2H]c1c([2H])c([2H])c(-c2ccc3c(ccc4c(-c5c6ccccc6c(-c6c7oc8ccccc8c7cc7c6oc6ccccc67)c6ccccc56)cccc43)c2)c([2H])c1[2H]]

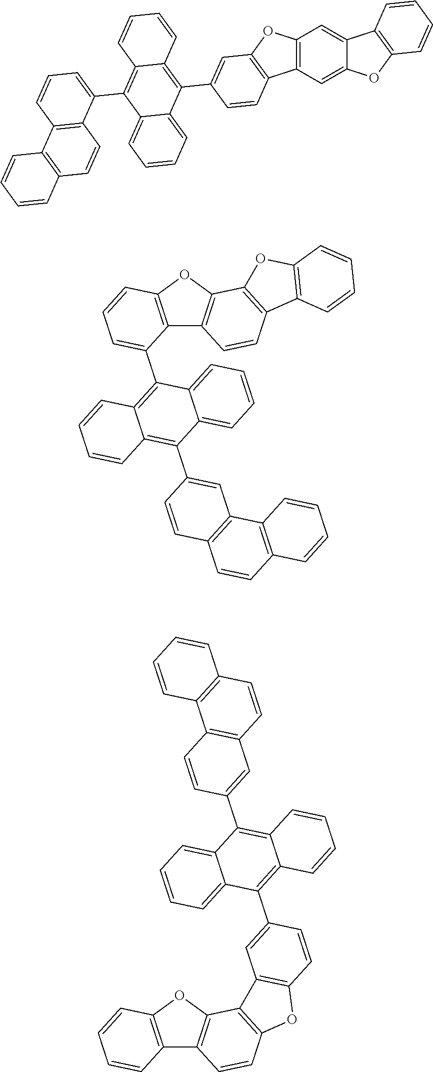 [SMILES: c1ccc2c(c1)ccc1c(-c3c4ccccc4c(-c4ccc5c(c4)oc4cc6c(cc45)oc4ccccc46)c4ccccc34)cccc12 ; c1ccc2c(c1)ccc1ccc(-c3c4ccccc4c(-c4cccc5oc6c(ccc7c8ccccc8oc76)c45)c4ccccc34)cc12 ; c1ccc2c(c1)ccc1cc(-c3c4ccccc4c(-c4ccc5oc6ccc7c8ccccc8oc7c6c5c4)c4ccccc34)ccc12]

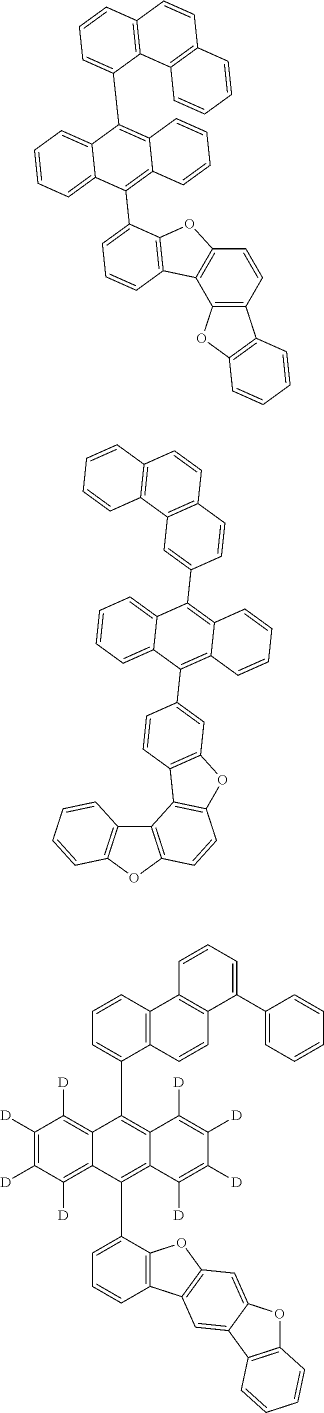 [SMILES: c1ccc2c(c1)ccc1cccc(-c3c4ccccc4c(-c4cccc5c4oc4ccc6c7ccccc7oc6c45)c4ccccc34)c12 ; c1ccc2c(c1)ccc1ccc(-c3c4ccccc4c(-c4ccc5c(c4)oc4ccc6oc7ccccc7c6c45)c4ccccc34)cc12 ; [2H]c1c([2H])c([2H])c2c(-c3cccc4c3oc3cc5oc6ccccc6c5cc34)c3c([2H])c([2H])c([2H])c([2H])c3c(-c3cccc4c3ccc3c(-c5ccccc5)cccc34)c2c1[2H]]

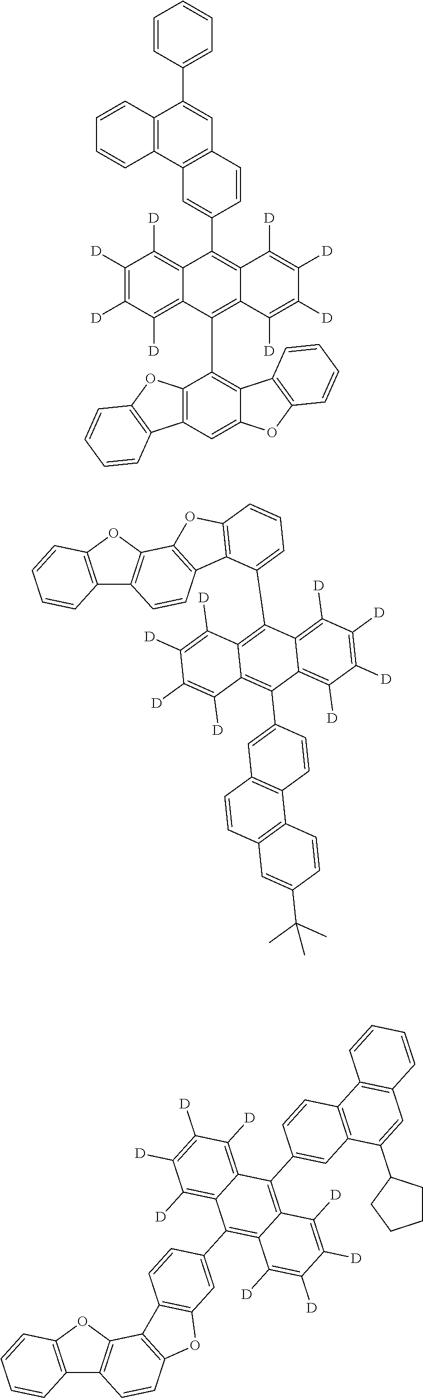 [SMILES: [2H]c1c([2H])c([2H])c2c(-c3c4oc5ccccc5c4cc4oc5ccccc5c34)c3c([2H])c([2H])c([2H])c([2H])c3c(-c3ccc4cc(-c5ccccc5)c5ccccc5c4c3)c2c1[2H] ; [2H]c1c([2H])c([2H])c2c(-c3cccc4oc5c(ccc6c7ccccc7oc65)c34)c3c([2H])c([2H])c([2H])c([2H])c3c(-c3ccc4c(ccc5cc(C(C)(C)C)ccc54)c3)c2c1[2H] ; [2H]c1c([2H])c([2H])c2c(-c3ccc4c(c3)c(C3CCCC3)cc3ccccc34)c3c([2H])c([2H])c([2H])c([2H])c3c(-c3ccc4c(c3)oc3ccc5c6ccccc6oc5c34)c2c1[2H]]

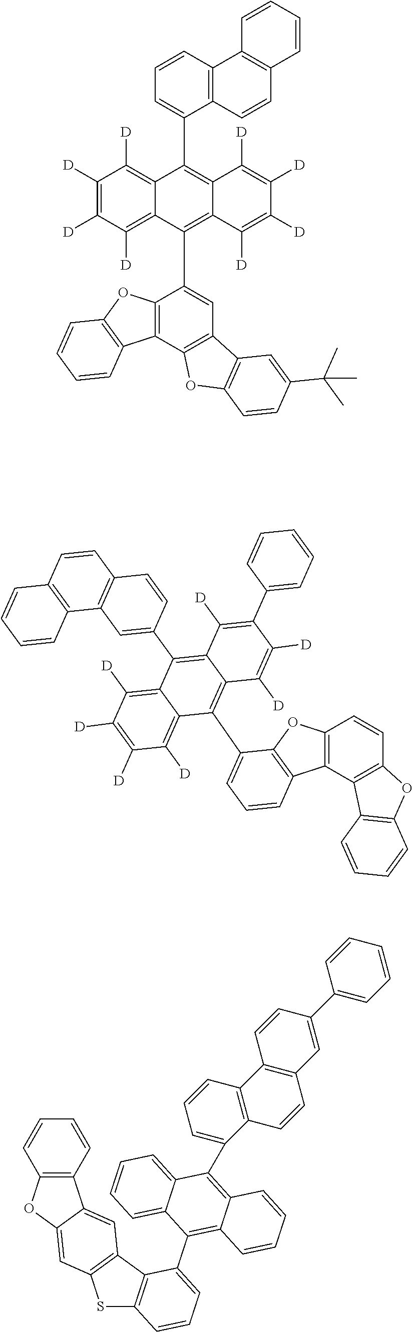 [SMILES: [2H]c1c([2H])c([2H])c2c(-c3cc4c5cc(C(C)(C)C)ccc5oc4c4c3oc3ccccc34)c3c([2H])c([2H])c([2H])c([2H])c3c(-c3cccc4c3ccc3ccccc34)c2c1[2H] ; [2H]c1c([2H])c([2H])c2c(-c3cccc4c3oc3ccc5oc6ccccc6c5c34)c3c([2H])c([2H])c(-c4ccccc4)c([2H])c3c(-c3ccc4ccc5ccccc5c4c3)c2c1[2H] ; c1ccc(-c2ccc3c(ccc4c(-c5c6ccccc6c(-c6cccc7sc8cc9oc%10ccccc%10c9cc8c67)c6ccccc56)cccc43)c2)cc1]

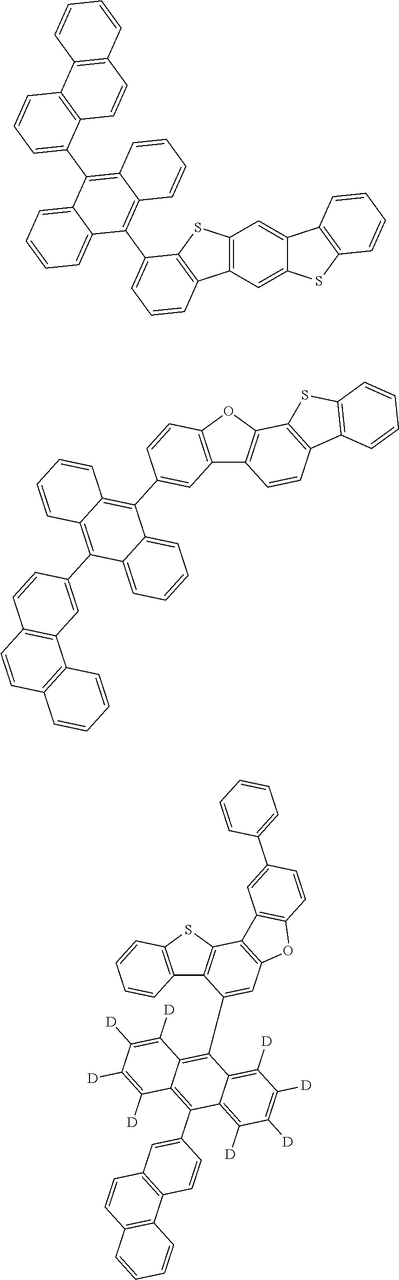 [SMILES: c1ccc2c(c1)ccc1c(-c3c4ccccc4c(-c4cccc5c4sc4cc6c(cc45)sc4ccccc46)c4ccccc34)cccc12 ; c1ccc2c(c1)ccc1ccc(-c3c4ccccc4c(-c4ccc5oc6c(ccc7c8ccccc8sc76)c5c4)c4ccccc34)cc12 ; [2H]c1c([2H])c([2H])c2c(-c3cc4oc5ccc(-c6ccccc6)cc5c4c4sc5ccccc5c34)c3c([2H])c([2H])c([2H])c([2H])c3c(-c3ccc4c(ccc5ccccc54)c3)c2c1[2H]]

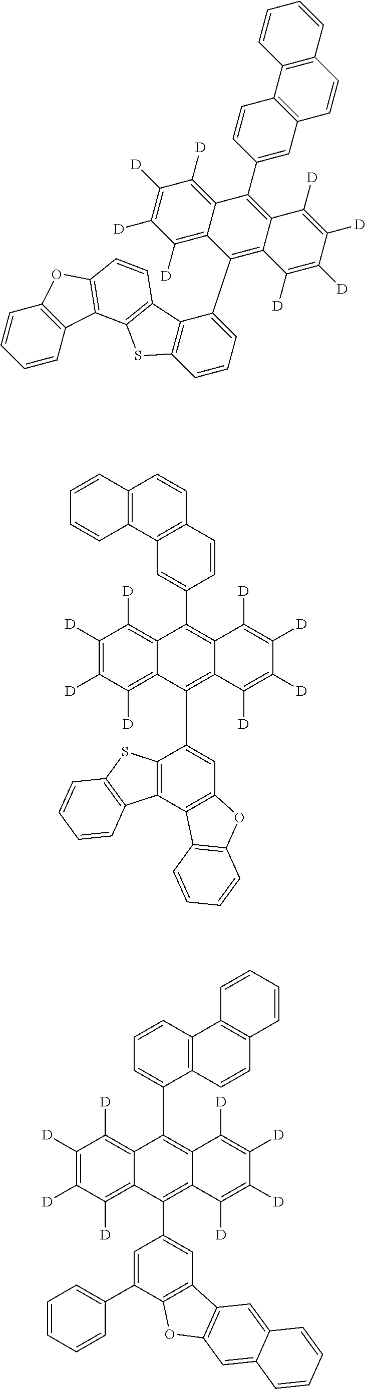 [SMILES: [2H]c1c([2H])c([2H])c2c(-c3cccc4sc5c(ccc6oc7ccccc7c65)c34)c3c([2H])c([2H])c([2H])c([2H])c3c(-c3ccc4c(ccc5ccccc54)c3)c2c1[2H] ; [2H]c1c([2H])c([2H])c2c(-c3cc4oc5ccccc5c4c4c3sc3ccccc34)c3c([2H])c([2H])c([2H])c([2H])c3c(-c3ccc4ccc5ccccc5c4c3)c2c1[2H] ; [2H]c1c([2H])c([2H])c2c(-c3cccc4c3ccc3ccccc34)c3c([2H])c([2H])c([2H])c([2H])c3c(-c3cc(-c4ccccc4)c4oc5cc6ccccc6cc5c4c3)c2c1[2H]]

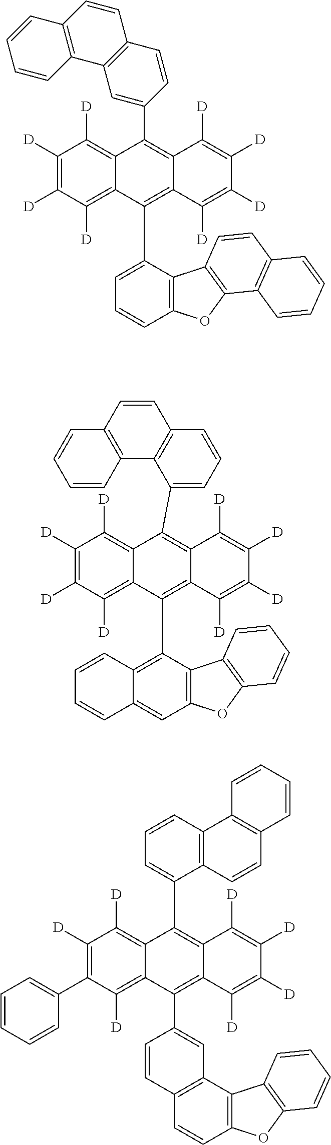 [SMILES: [2H]c1c([2H])c([2H])c2c(-c3cccc4oc5c6ccccc6ccc5c34)c3c([2H])c([2H])c([2H])c([2H])c3c(-c3ccc4ccc5ccccc5c4c3)c2c1[2H] ; [2H]c1c([2H])c([2H])c2c(-c3c4ccccc4cc4oc5ccccc5c34)c3c([2H])c([2H])c([2H])c([2H])c3c(-c3cccc4ccc5ccccc5c34)c2c1[2H] ; [2H]c1c([2H])c([2H])c2c(-c3cccc4c3ccc3ccccc34)c3c([2H])c([2H])c(-c4ccccc4)c([2H])c3c(-c3ccc4ccc5oc6ccccc6c5c4c3)c2c1[2H]]

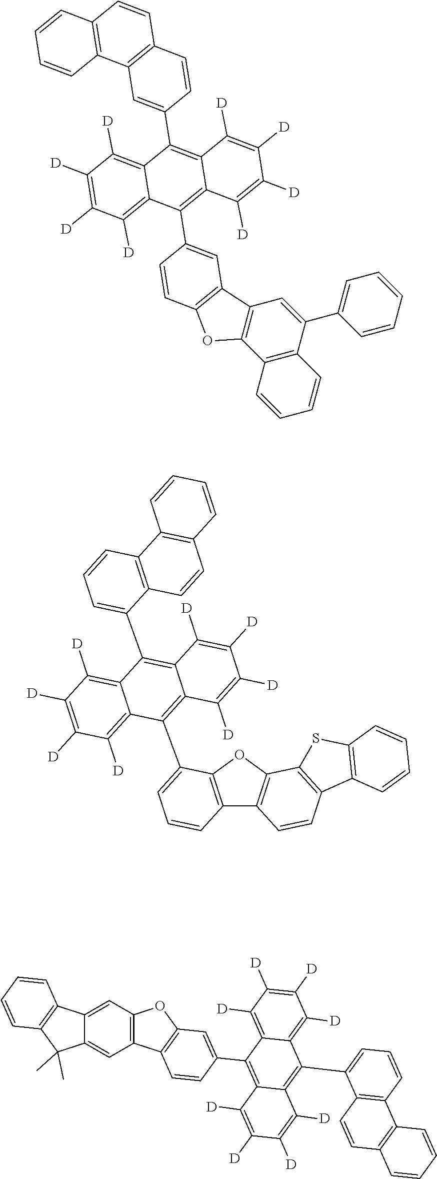 [SMILES: [2H]c1c([2H])c([2H])c2c(-c3ccc4oc5c6ccccc6c(-c6ccccc6)cc5c4c3)c3c([2H])c([2H])c([2H])c([2H])c3c(-c3ccc4ccc5ccccc5c4c3)c2c1[2H] ; [2H]c1c([2H])c([2H])c2c(-c3cccc4c3oc3c4ccc4c5ccccc5sc43)c3c([2H])c([2H])c([2H])c([2H])c3c(-c3cccc4c3ccc3ccccc34)c2c1[2H] ; [2H]c1c([2H])c([2H])c2c(-c3cccc4c3ccc3ccccc34)c3c([2H])c([2H])c([2H])c([2H])c3c(-c3ccc4c(c3)oc3cc5c(cc34)C(C)(C)c3ccccc3-5)c2c1[2H]]

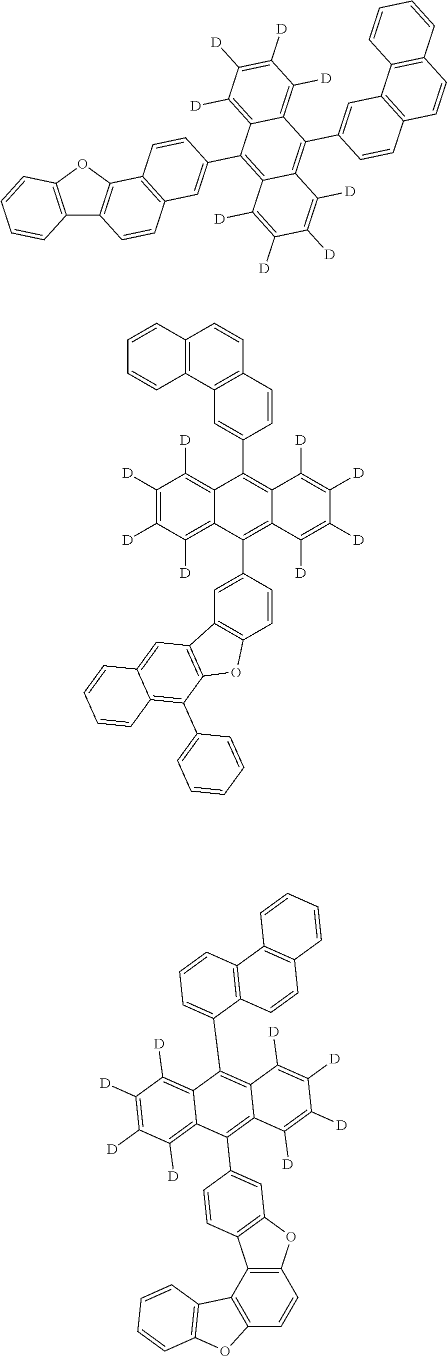 [SMILES: [2H]c1c([2H])c([2H])c2c(-c3ccc4ccc5ccccc5c4c3)c3c([2H])c([2H])c([2H])c([2H])c3c(-c3ccc4c(ccc5c6ccccc6oc45)c3)c2c1[2H] ; [2H]c1c([2H])c([2H])c2c(-c3ccc4oc5c(-c6ccccc6)c6ccccc6cc5c4c3)c3c([2H])c([2H])c([2H])c([2H])c3c(-c3ccc4ccc5ccccc5c4c3)c2c1[2H] ; [2H]c1c([2H])c([2H])c2c(-c3cccc4c3ccc3ccccc34)c3c([2H])c([2H])c([2H])c([2H])c3c(-c3ccc4c(c3)oc3ccc5oc6ccccc6c5c34)c2c1[2H]]

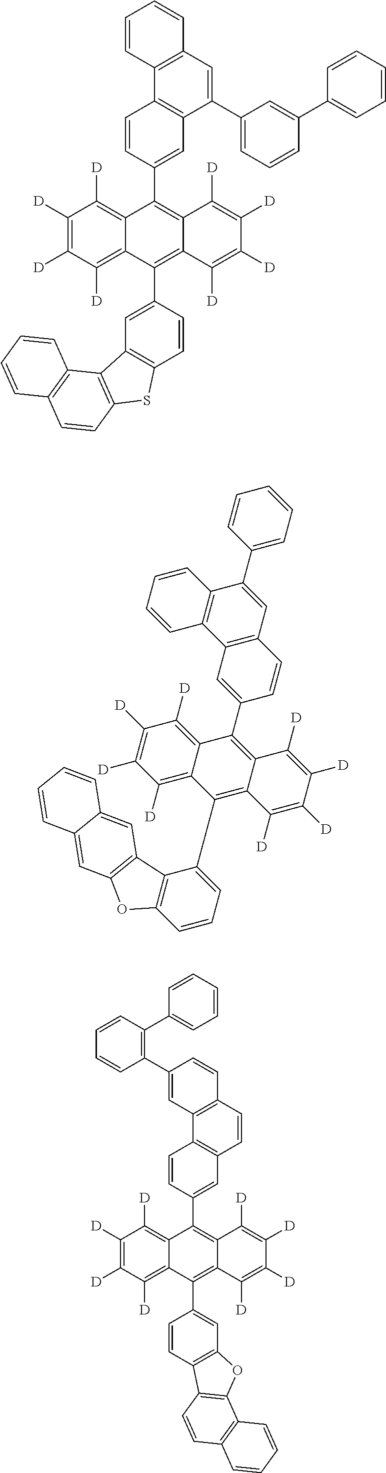

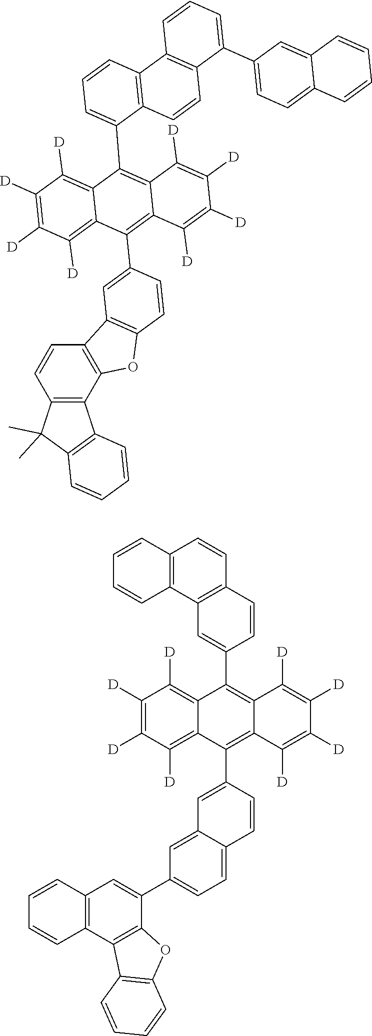 [SMILES: [2H]c1c([2H])c([2H])c2c(-c3cccc4c3ccc3c(-c5ccc6ccccc6c5)cccc34)c3c([2H])c([2H])c([2H])c([2H])c3c(-c3ccc4oc5c6c(ccc5c4c3)C(C)(C)c3ccccc3-6)c2c1[2H] ; [2H]c1c([2H])c([2H])c2c(-c3ccc4ccc5ccccc5c4c3)c3c([2H])c([2H])c([2H])c([2H])c3c(-c3ccc4ccc(-c5cc6ccccc6c6c5oc5ccccc56)cc4c3)c2c1[2H]]

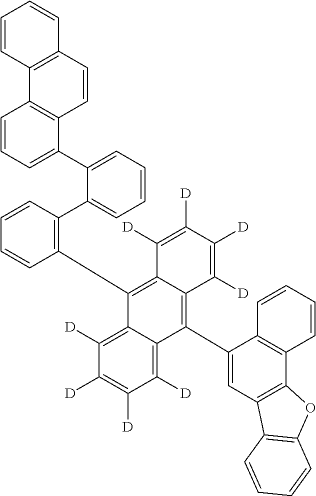 [SMILES: [2H]c1c([2H])c([2H])c2c(-c3cc4c5ccccc5oc4c4ccccc34)c3c([2H])c([2H])c([2H])c([2H])c3c(-c3ccccc3-c3ccccc3-c3cccc4c3ccc3ccccc34)c2c1[2H]]

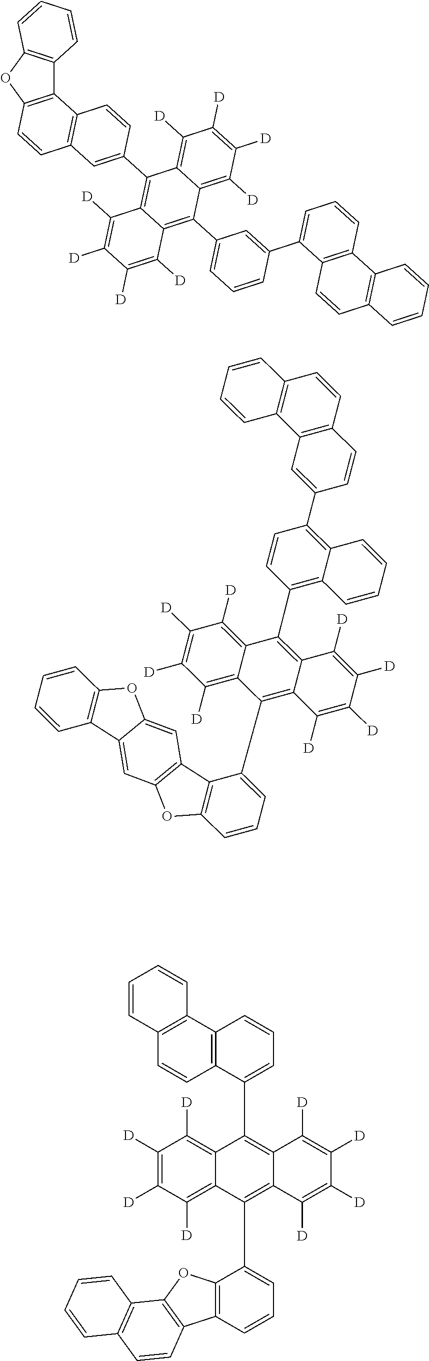 [SMILES: [2H]c1c([2H])c([2H])c2c(-c3ccc4c(ccc5oc6ccccc6c54)c3)c3c([2H])c([2H])c([2H])c([2H])c3c(-c3cccc(-c4cccc5c4ccc4ccccc45)c3)c2c1[2H] ; [2H]c1c([2H])c([2H])c2c(-c3cccc4oc5cc6c(cc5c34)oc3ccccc36)c3c([2H])c([2H])c([2H])c([2H])c3c(-c3ccc(-c4ccc5ccc6ccccc6c5c4)c4ccccc34)c2c1[2H] ; [2H]c1c([2H])c([2H])c2c(-c3cccc4c3oc3c5ccccc5ccc43)c3c([2H])c([2H])c([2H])c([2H])c3c(-c3cccc4c3ccc3ccccc34)c2c1[2H]]

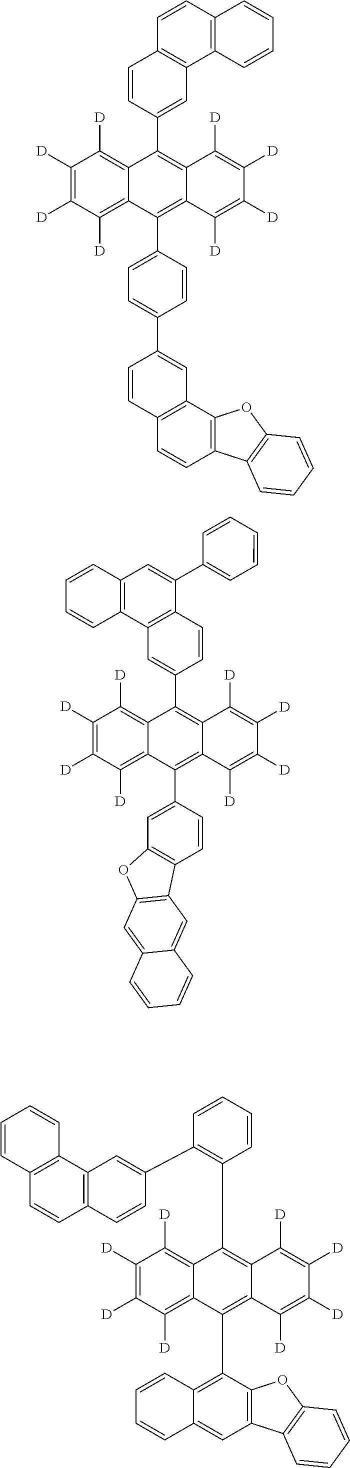

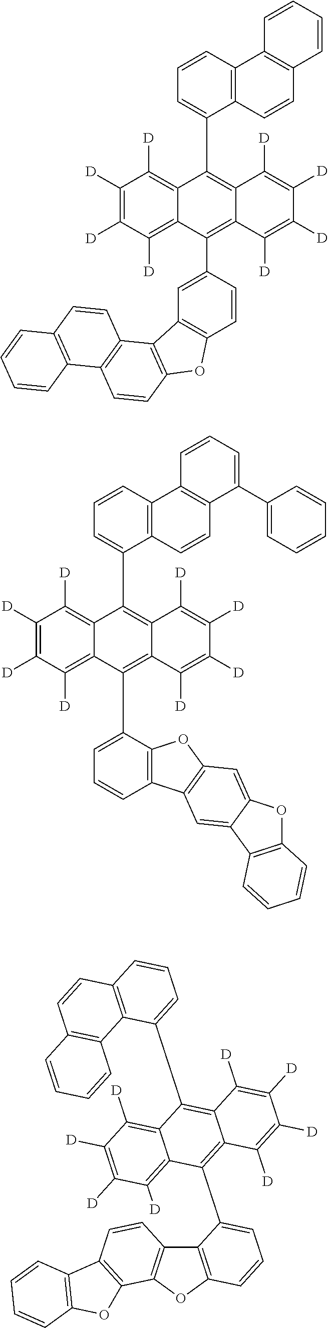 [SMILES: [2H]c1c([2H])c([2H])c2c(-c3cccc4c3ccc3ccccc34)c3c([2H])c([2H])c([2H])c([2H])c3c(-c3ccc4oc5ccc6c7ccccc7ccc6c5c4c3)c2c1[2H] ; [2H]c1c([2H])c([2H])c2c(-c3cccc4c3oc3cc5oc6ccccc6c5cc34)c3c([2H])c([2H])c([2H])c([2H])c3c(-c3cccc4c3ccc3c(-c5ccccc5)cccc34)c2c1[2H] ; [2H]c1c([2H])c([2H])c2c(-c3cccc4oc5c(ccc6c7ccccc7oc65)c34)c3c([2H])c([2H])c([2H])c([2H])c3c(-c3cccc4ccc5ccccc5c34)c2c1[2H]]

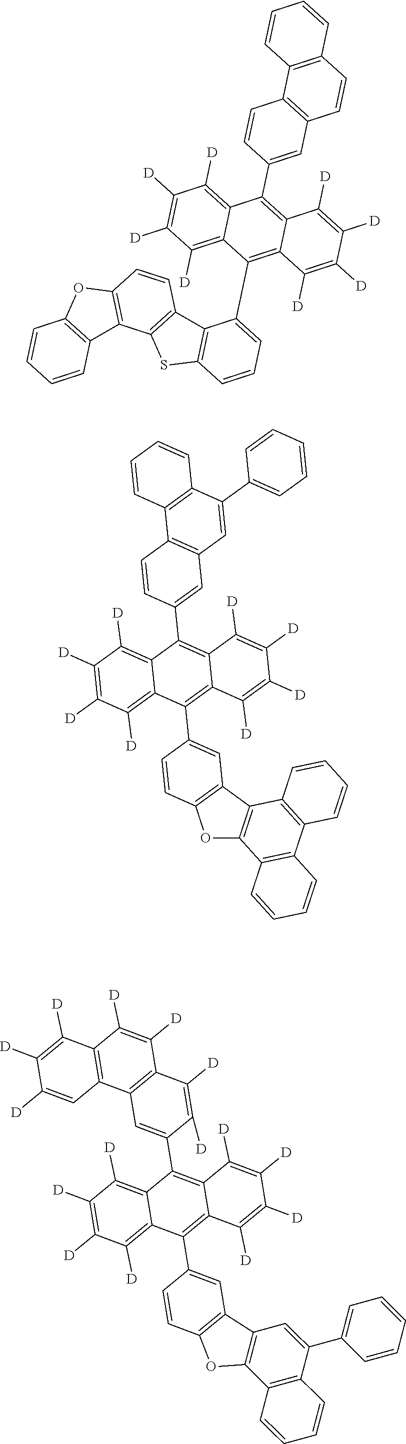 [SMILES: [2H]c1c([2H])c([2H])c2c(-c3cccc4sc5c(ccc6oc7ccccc7c65)c34)c3c([2H])c([2H])c([2H])c([2H])c3c(-c3ccc4c(ccc5ccccc54)c3)c2c1[2H] ; [2H]c1c([2H])c([2H])c2c(-c3ccc4oc5c6ccccc6c6ccccc6c5c4c3)c3c([2H])c([2H])c([2H])c([2H])c3c(-c3ccc4c(c3)cc(-c3ccccc3)c3ccccc34)c2c1[2H] ; [2H]c1cc2c(c([2H])c1[2H])c([2H])c([2H])c1c([2H])c([2H])c(-c3c4c([2H])c([2H])c([2H])c([2H])c4c(-c4ccc5oc6c7ccccc7c(-c7ccccc7)cc6c5c4)c4c([2H])c([2H])c([2H])c([2H])c34)cc12]

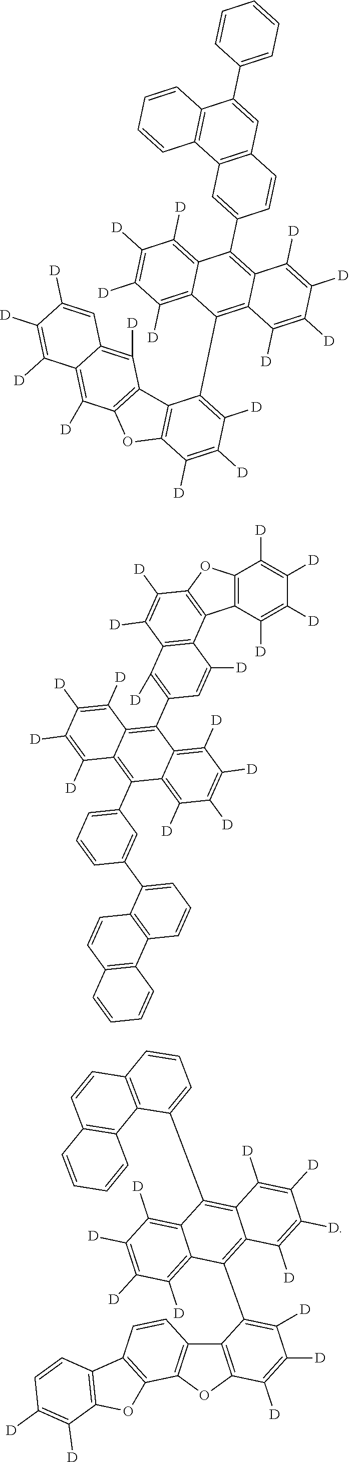

9. An organic light emitting device comprising:
an anode;
a cathode; and
an organic material layer having one or more layers provided between the anode and the cathode,
wherein one or more layers of the organic material layer comprise the organic compound according to claim 1.

10. The organic light emitting device of claim 9, wherein the organic material layer comprises a light emitting layer, and the light emitting layer comprises the organic compound.

11. The organic light emitting device of claim 10, wherein the light emitting layer comprises the organic compound as a host of the light emitting layer.

12. The organic light emitting device of claim 11, wherein the light emitting layer comprises the organic compound as a host and further comprises one host represented by the following Chemical Formula H-1 different from the organic compound: 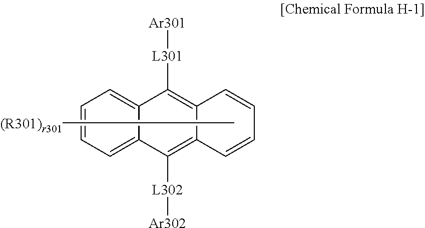
in Chemical Formula H-1,
L301 and L302 are the same as or different from each other, and are each independently a direct bond; a substituted or unsubstituted arylene group; or a substituted or unsubstituted heterocyclic group,
Ar301 and Ar302 are the same as or different from each other, and are each independently hydrogen; deuterium; a substituted or unsubstituted aryl group; or a substituted or unsubstituted heterocyclic group,
R301 is hydrogen; deuterium; a halogen group; a substituted or unsubstituted alkyl group; a substituted or unsubstituted cycloalkyl group; a substituted or unsubstituted aryl group; or a substituted or unsubstituted heterocyclic group, and
r301 is an integer from 1 to 7, and when r301 is 2 or higher, two or more R301's are the same as or different from each other.

13. The organic light emitting device of claim 10, wherein the light emitting layer comprises the organic compound as a host, and further comprises a dopant.
